SEQUENCE LISTING (58 sequences)

SEQ ID NO: 1 - protein, 8 aa, Homo sapiens
GAQSPQNC

SEQ ID NO: 2 - protein, 10 aa, Homo sapiens
GSGAQSPQNC

SEQ ID NO: 3 - protein, 13 aa, Homo sapiens
GSGAQSPQNCSIM

SEQ ID NO: 4 - DNA, 25 bp, Artificial
  synthetic oligonucleotide
ccggaattctatggagaccccgtcc

SEQ ID NO: 5 - DNA, 22 bp, Artificial
  synthetic oligonucleotide
cggggtacccatgatgctgcag

SEQ ID NO: 6 - DNA, 33 bp, Artificial
  synthetic oligonucleotide
ggagcccagagcccccagaactgcagcatcatg

SEQ ID NO: 7 - DNA, 19 bp, Artificial
  synthetic oligonucleotide
ggagcccagagcccccaga

SEQ ID NO: 8 - DNA, 19 bp, Artificial
  synthetic oligonucleotide
gagcccagagcccccagaa

SEQ ID NO: 9 - DNA, 19 bp, Artificial
  synthetic oligonucleotide
agcccagagcccccagaac

SEQ ID NO: 10 - DNA, 19 bp, Artificial
  synthetic oligonucleotide
gcccagagcccccagaact

SEQ ID NO: 11 - DNA, 19 bp, Artificial
  synthetic oligonucleotide
cccagagcccccagaactg

SEQ ID NO: 12 - DNA, 19 bp, Artificial
  synthetic oligonucleotide
ccagagcccccagaactgc

SEQ ID NO: 13 - DNA, 19 bp, Artificial
  synthetic oligonucleotide
cagagcccccagaactgca

SEQ ID NO: 14 - DNA, 19 bp, Artificial
  synthetic oligonucleotide
agagcccccagaactgcag

SEQ ID NO: 15 - DNA, 19 bp, Artificial
  synthetic oligonucleotide
gagcccccagaactgcagc

SEQ ID NO: 16 - DNA, 19 bp, Artificial
  synthetic oligonucleotide
agcccccagaactgcagca

SEQ ID NO: 17 - DNA, 19 bp, Artificial
  synthetic oligonucleotide
gcccccagaactgcagcat

SEQ ID NO: 18 - DNA, 19 bp, Artificial
  synthetic oligonucleotide
cccccagaactgcagcatc

SEQ ID NO: 19 - DNA, 19 bp, Artificial
  synthetic oligonucleotide
ccccagaactgcagcatca

SEQ ID NO: 20 - DNA, 19 bp, Artificial
  synthetic oligonucleotide
cccagaactgcagcatcat

SEQ ID NO: 21 - DNA, 19 bp, Artificial
  synthetic oligonucleotide
ccagaactgcagcatcatg

SEQ ID NO: 22 - DNA, 20 bp, Artificial
  synthetic oligonucleotide
ggagcccagagcccccagaa

SEQ ID NO: 23 - DNA, 20 bp, Artificial
  synthetic oligonucleotide
gagcccagagcccccagaac

SEQ ID NO: 24 - DNA, 20 bp, Artificial
  synthetic oligonucleotide
agcccagagcccccagaact

SEQ ID NO: 25 - DNA, 20 bp, Artificial
  synthetic oligonucleotide
gcccagagcccccagaactg

SEQ ID NO: 26 - DNA, 20 bp, Artificial
  synthetic oligonucleotide
cccagagcccccagaactgc

SEQ ID NO: 27 - DNA, 20 bp, Artificial
  synthetic oligonucleotide
ccagagcccccagaactgca

SEQ ID NO: 28 - DNA, 20 bp, Artificial
  synthetic oligonucleotide
cagagcccccagaactgcag

SEQ ID NO: 29 - DNA, 20 bp, Artificial
  synthetic oligonucleotide
agagcccccagaactgcagc

SEQ ID NO: 30 - DNA, 20 bp, Artificial
  synthetic oligonucleotide
gagcccccagaactgcagca

SEQ ID NO: 31 - DNA, 20 bp, Artificial
  synthetic oligonucleotide
agcccccagaactgcagcat

SEQ ID NO: 32 - DNA, 20 bp, Artificial
  synthetic oligonucleotide
gcccccagaactgcagcatc

SEQ ID NO: 33 - DNA, 20 bp, Artificial
  synthetic oligonucleotide
cccccagaactgcagcatca

SEQ ID NO: 34 - DNA, 20 bp, Artificial
  synthetic oligonucleotide
ccccagaactgcagcatcat

SEQ ID NO: 35 - DNA, 20 bp, Artificial
  synthetic oligonucleotide
cccagaactgcagcatcatg

SEQ ID NO: 36 - DNA, 21 bp, Artificial
  synthetic oligonucleotide
ggagcccagagcccccagaac

SEQ ID NO: 37 - DNA, 21 bp, Artificial
  synthetic oligonucleotide
gagcccagagcccccagaact

SEQ ID NO: 38 - DNA, 21 bp, Artificial
  synthetic oligonucleotide
agcccagagcccccagaactg

SEQ ID NO: 39 - DNA, 21 bp, Artificial
  synthetic oligonucleotide
gcccagagcccccagaactgc

SEQ ID NO: 40 - DNA, 21 bp, Artificial
  synthetic oligonucleotide
cccagagcccccagaactgca

SEQ ID NO: 41 - DNA, 21 bp, Artificial
  synthetic oligonucleotide
ccagagcccccagaactgcag

SEQ ID NO: 42 - DNA, 21 bp, Artificial
  synthetic oligonucleotide
cagagcccccagaactgcagc

SEQ ID NO: 43 - DNA, 21 bp, Artificial
  synthetic oligonucleotide
agagcccccagaactgcagca

SEQ ID NO: 44 - DNA, 21 bp, Artificial
  synthetic oligonucleotide
gagcccccagaactgcagcat

SEQ ID NO: 45 - DNA, 21 bp, Artificial
  synthetic oligonucleotide
agcccccagaactgcagcatc

SEQ ID NO: 46 - DNA, 21 bp, Artificial
  synthetic oligonucleotide
gcccccagaactgcagcatca

SEQ ID NO: 47 - DNA, 21 bp, Artificial
  synthetic oligonucleotide
cccccagaactgcagcatcat

SEQ ID NO: 48 - DNA, 21 bp, Artificial
  synthetic oligonucleotide
ccccagaactgcagcatcatg

SEQ ID NO: 49 - protein, 11 aa, Homo sapiens
SGSGAQSPQNC

SEQ ID NO: 50 - protein, 14 aa, Homo sapiens
SGSGAQSPQNCSIM

SEQ ID NO: 51 - DNA, 23 bp, Artificial
  synthetic oligonucleotide
aannnnnnnnnnnnnnnnnnnuu

SEQ ID NO: 52 - DNA, 24 bp, Artificial
  synthetic oligonucleotide
aannnnnnnnnnnnnnnnnnnnuu

SEQ ID NO: 53 - DNA, 25 bp, Artificial
  synthetic oligonucleotide
aannnnnnnnnnnnnnnnnnnnnuu

SEQ ID NO: 54 - DNA, 39 bp, Homo sapiens
ggctcaggagcccagagcccccagaactgcagcatcatg

SEQ ID NO: 55 - DNA, 15 bp, Artificial
  synthetic oligonucleotide
ggctgcgggaacagc

SEQ ID NO: 56 - DNA, 12 bp, Artificial
  synthetic oligonucleotide
ctggcaggtccc

SEQ ID NO: 57 - DNA, 20 bp, Artificial
  synthetic oligonucleotide
cccagcacaatgaagatcaa

SEQ ID NO: 58 - DNA, 20 bp, Artificial
  synthetic oligonucleotide
gtgtaacgcaactaagtcat

(19) United States
(12) Patent Application Publication
(10) Pub. No.: US 2008/0292617 A1
(43) Pub. Date: Nov. 27, 2008

(54) ANTIBODY AGAINST A PEPTIDE DERIVED FROM HUMAN MUTANT LAMIN A PROTEIN AND USES THEREOF

(75) Inventor: Karima DJABALI, New York, NY (US)

Correspondence Address: WilmerHale/Columbia University, 399 PARK AVENUE, NEW YORK, NY, 10022, US

(73) Assignee: THE TRUSTEES OF COLUMBIA UNIVERSITY IN THE CITY OF NEW YORK, New York, NY (US)

(21) Appl. No.: 12/101,651

(22) Filed: Apr. 11, 2008

Related U.S. Application Data
(63) Continuation-in-part of application No. PCT/US2006/040279, filed on Oct. 13, 2006.
(60) Provisional application No. 60/726,260, filed on Oct. 13, 2005.

(51) Int. Cl. A61K 39/395; C07K 7/08; C07K 16/00; G01N 33/567; G01N 33/566; C07H 21/04; A61K 31/711; C12N 15/63; C12N 5/02

(52) U.S. Cl. 424/130.1; 530/328; 530/387.9; 435/7.2; 435/7.21; 436/501; 536/24.5; 514/44; 435/320.1; 435/375

(57) ABSTRACT

Nearly all subjects affected with Hutchinson Gilford progeria syndrome (HGPS) carry mutation LMNA G608G (GGC>GGT), within exon 11 of LMNA activating a splicing donor site which leads to a deletion of 50 amino acids at the carboxyl-terminal of prelamin A. The invention provides an isolated peptide of sequence GAQSPQNC. The lamin A G608G polypeptide comprises the GAQSPQNC peptide. The invention provides monoclonal and polyclonal antibodies, which specifically recognize GAQSPQNC peptide, and any polypeptide which comprises GAQSPQNC. The invention provides methods, which use the inventive antibodies to detect biological conditions associated with lamin A G608G; and to identify agents which inhibit expression or localization of lamin A G608G.

This application is a continuation-in-part of International Application No. PCT/US2006/040279 filed on Oct. 13, 2006, which claims the benefit of priority of U.S. Ser. No. 60/726,260 filed on Oct. 13, 2005, the contents of which are hereby incorporated in their entirety.

The invention was made with government support under R03AR47641 and RO1AG025302 awarded by the National Institutes of Health. The government has certain rights in the invention.

This patent disclosure contains material that is subject to copyright protection. The copyright owner has no objection to the facsimile reproduction by anyone of the patent document or the patent disclosure, as it appears in the U.S. Patent and Trademark Office patent file or records, but otherwise reserves any and all copyright rights.

Throughout this application, patent applications, published patent applications, issued and granted patents, texts, and literature references are cited. The disclosures of these publications in their entireties are hereby incorporated by reference into this application in order to more fully describe the state of the art as known to those skilled therein as of the date of the invention described and claimed herein.

### BACKGROUND

The Hutchinson Gilford progeria syndrome (HGPS, OMIM 176670) is a rare sporadic disorder with an incidence of 1 per 8 million live births, comprising a premature aging phenotype with rapid growth deceleration in childhood. In this syndrome, the appearance at birth and the birth weight are can be normal, but growth typically starts to become retarded at the age of 1 year. The phenotypic appearances consist of the following features: short stature, sculptured nose, alopecia, prominent scalp veins, small face, loss of subcutaneous fat, faint mid-facial cyanosis, and dystrophic nails. In addition, HGPS patients show skeletal abnormalities that can reflect a deficit in osteogenesis, mainly in extremities, mandibular and cranial dysplasia with disorganized growth, deformability in dentition, as well as severe osteolysis (Fernandez et al., 1992; Sweeney and Weiss, 1992). The common causes of death in HGPS subjects during the second decade of life are due to chronic conditions common inelderly people mainly coronary artery disease and stroke due to widespread atherosclerosis (Brown et al., 1992). Features associated with pathological aging such as increased prevalence of some types of cancer, dementia, cataracts, and diabetes have not been seen (Mills and Weiss, 1990). Most abnormalities described in HGPS are the disorders of mesodermal tissues.

Until recently, the diagnosis of HGPS was mainly based on the criteria of growth retardation and the prematurely aged appearance in children. In 2003 the causative gene of HGPS was identified as the LMNA gene, (Cao and Hegele, 2003; De Sandre-Giovannoli et al., 2003; Eriksson et al., 2003). Out of 22 HGPS DNA samples, screened for LMNA, 20 have a heterozygous base substitution G608G (GGC>GGT) within exon 11 (Cao and Hegele, 2003; De Sandre-Giovannoli et al., 2003; Eriksson et al., 2003 and a mutation described herein). In one sample, another heterozygous substitution was found in the same codon G608S (GGC>AGC) (Eriksson et al., 2003). None of the parents carried this de novo G608G point mutation, suggesting that HGPS is a dominantly inherited disease probably due to a germ line mosaicism. The fact that G608G appears to be the common mutation for the majority of HGPS makes the screening of the LMNA exon 11 an assay tool to ascertain the HGPS diagnosis very early on, before the complete clinical phenotype has become obvious.

### SUMMARY OF THE INVENTION

The invention provides a peptide having amino acid sequence GAQSPQNC or having an amino sequence which is at least 75%, 80%, 85%, 90%, 95%, and 99.9% identical to the amino acid sequence GAQSPQNC (SEQ ID NO: 1). The invention provides a peptide having amino acid sequence GSGAQSPQNC or having an amino sequence which is at least 75%, 80%, 85%, 90%, 95%, and 99.9% identical to the amino acid sequence GSGAQSPQNC (SEQ ID NO: 2). The invention provides a peptide having amino acid sequence GSGAQSPQNCSIM or having an amino sequence which is at least 75%, 80%, 85%, 90%, 95%, and 99.9% identical to the amino acid sequence GSGAQSPQNCSIM (SEQ ID NO: 3). The invention provides a peptide having amino acid sequence of SEQ ID NO: 1, 2, or 3, or having an amino acid sequence which is at least about 70%, 72%, 74%, 76%, 78%, 80%, 82%, 84%, 86%, 88%, 90%, 91%, 92%, 93%, 94%, 95%, 96%, 97%, 98%, 99%, 99.5% identical to the amino acid sequence set forth in SEQ ID NO:1, 2, or 3. In certain aspects, the peptide of the invention is isolated. In certain aspects, the invention provides a mixture comprising at least 2 of the inventive peptides. In certain aspects, the invention provides a mixture comprising about 2, 5, 10, 15, 20, 25, 30, 35, 40, 50, 60, 70, 80, 90, 100 of the inventive peptides. In certain aspects, the invention provides a mixture consisting essentially of about 2, 5, 10, 15, 20, 25, 30, 35, 40, 50, 60, 70, 80, 90, 100 of the inventive peptides.

In other aspects, the invention provides modified peptides, peptides which are conjugated or coupled to carries, peptides which are modified so as to have increased stability when administered in a subject, and peptidomimetics with increased stability. In other aspects, the invention provides peptides conjugated to various detectable marker, including but not limited to fluorescently labeled, or radiolabeled markers.

The invention provides an antibody that specifically binds to the peptide of SEQ ID NO: 1, or a polypeptide which comprises the amino acid sequence of SEQ ID NO: 1. In certain aspects, the inventive antibody is a polyclonal antibody. In a specific embodiment, the polyclonal antibody is from a rabbit. In other aspects, the inventive antibody is a monoclonal antibody. In another aspect, the inventive antibody is a soluble F(ab′)₂ fragment which specifically binds to the peptide of SEQ ID NO: 1, or a polypeptide which comprises the amino acid sequence of SEQ ID NO: 1.

The invention provides a method for detecting a biological condition associated with a lamin A G608G mutation in a subject, the method comprising determining the presence of a mutant lamin A G608G protein. In certain aspects, the invention provides a method for detecting a biological condition associated with a lamin A G608G mutation in a subject, the method comprising determining whether an antibody that specifically binds to lamin A G608G protein binds to a sample from the subject, wherein binding of the antibody indicates the presence of mutant lamin A G608G. In certain aspects, binding is detected through Western blotting or immunohistochemistry. In certain aspects, the sample is a tissue section or biopsy from a subject. In other aspects, the sample is protein extract from a subject. In certain aspects, determining the presence of the lamin A G608G protein is detected by immunohistochemistry which uses an inventive antibody that specifically binds to the lamin A G608G protein. In certain aspect, the presence of the lamin A G608G protein is detected by immuno (western) blotting of cellular extracts derived from subjects suspected to be affected with a biological condition associated with dominant lamin A G608G mutation, wherein the method employs an inventive antibody that specifically binds to the lamin A G608G protein. In other aspects, the invention provides a kit for detecting a biological condition associated with the presence of a lamin A G608G mutation in a subject, the kit comprising the antibody of the invention, and a control reaction which can include the peptide of SEQ ID NO:1, and/or a polypeptide which comprise SEQ ID NO:1. Lamin A G608G is detected in HGPS patients and in some elderly subjects who do not suffer from HGPS.

The invention provides, methods for identifying an agent capable of modulating and/or inhibiting mutant lamin A G608G protein, the method comprising: a) contacting an agent with a cell expressing a mutant lamin A G608G protein, b) determining whether the cell exhibits an increased or a decreased level of the mutant lamin A G608G protein, wherein exhibition of decreased mutant lamin A protein level is indicative of an agent that decreases the level of the mutant lamin A protein. In certain aspects, the invention provides a method for selecting an agent capable of modulating and/or inhibiting mutant lamin A G608G protein, the method comprising: a) contacting an agent with a cell expressing a mutant lamin A G608G protein, b) selecting cells with improved or restored cellular functions, for example improved heat shock resistance, or cell motility, wherein improved or restored cellular function is indicative of an agent that decreases the level of the mutant lamin A protein.

In certain aspects, the invention provides a method for identifying an agent capable of inhibiting expression, stability, activity, and/or localization of mutant lamin A G608G protein in a cell, the method comprising: a) contacting a cell expressing a mutant lamin A G608G protein with an agent, b) determining whether the cell exhibits an increased or a decreased amount of the mutant lamin A G608G protein, wherein a decreased amount indicates that the agent inhibits expression of mutant lamin A protein.

The invention provides methods for identifying an agent capable of inhibiting expression of mutant lamin A, and thereby inhibiting atherosclerosis, the method comprising: a) contacting a cell expressing a mutant lamin A G608G protein with an agent, b) determining whether the cell exhibits an increased or a decreased amount of the mutant lamin A G608G protein, wherein a decreased amount indicates that the agent inhibits expression of mutant lamin A protein and that the agent inhibits atherosclerosis.

The invention provides methods for identifying an agent capable of inhibiting expression of mutant lamin A, and thereby reducing, preventing or attenuating ageing, the method comprising: a) contacting a cell expressing a mutant lamin A G608G protein with an agent, b) determining whether the cell exhibits an increased or a decreased amount of the mutant lamin A G608G protein, wherein a decreased amount indicates that the agent inhibits expression of mutant lamin A protein and that the agent reduces, prevents, or attenuates ageing.

In certain aspects, the agents identified by the methods of the invention are antagonist/inhibitors which attenuate, downregulate, and/or inhibit mutant lamin A G608G. An antagonist can be any of: peptides, including the peptides of the invention, small molecules, antibodies, polynucleotide compounds, such as but not limited to antisense polynucleotides, ribozymes, RNA interference molecules, triple helix polynucleotides and the like, where the nucleotide sequence of such compounds are related to the nucleotide sequences of DNA and/or RNA of lamin A G608G. The term “inhibits” as used herein encompasses any reduction in levels of mRNA, protein, or activity of lamin A G608G, or lamin A G608G mediated activity thereof.

The determining step of the methods which identify agents that modulate lamin A G608G activity can be carried out by any suitable assay known in the art, including any of the assays and methods described herein. In one aspect, the determining step comprises detection by any immunoassay which uses the inventive antibody which specifically binds to lamin A G608G. In another aspect, the determining step can use immunohistochemistry which uses an antibody that specifically binds to the lamin A G608G protein. In another aspect, determination can further comprise quantitation of the strength of a fluorescent signal detected by immunohistochemistry which uses an antibody that specifically binds to the lamin A G608G protein. In another aspect, the determining step comprises detection by western blotting which uses an antibody that specifically binds to the lamin A G608G protein. Additional assays that can be used to identify an agent that modulates lamin A G608G activity include but are not limited to functional assays such as an assay that measures senescence of lamin A G608G cells in the presence or absence of an agent, an assay that measures Insulin growth factor binding protein (IGFBP-3) levels in the culture medium derived from lamin A G608G cells in the presence or absence of an agent, an assay that determines cell migration of lamin A G608G cells in the presence or absence of an agent.

Any suitable cell and/cell line which expresses lamin A G608G can be used in the methods which identify agents that modulate lamin A G608G activity. In certain aspects, the cell is derived from a subject who is suffering from HGPS. In other aspect, the cell is from a HPGS dermal fibroblast cell line. In other aspects, the cell line is an endothelial cell line from HGPS subject. In another aspect, the cell line can be any suitable cell line which is transfected to express, stably or transiently, lamin A G608G. In other aspect, the cell which expresses mutant lamin A G608G is an endothelial cell.

The invention provides methods for identifying a protein that interacts with mutant lamin A G608G, the method comprising: a) immunoprecipitating a protein interacting with mutant lamin A G608G, wherein immunoprecipitating is performed with the monoclonal or polyclonal antibody of the invention which specifically detects lamin A G608G, b) determining whether a protein immunoprecipitated with the antibody of the invention is identical or different compared to a protein immunoprecipitated with an antibody that immunoprecipitates lamin A, wherein the presence of a different protein in an immunoprecipitate with the antibody of indicates interaction with mutant lamin 1 G608G or the absence of a protein in an immunoprecipitate with the antibody of the invention indicates loss of interaction with mutant lamin A G608G.

The invention provides an isolated nucleic acid that encodes an inhibitory RNA molecule that inhibits the function, including but not limited to expression, activity and/or localization of mutant lamin A G608G. The invention provides a nucleic acid encoding an inhibitory RNA molecule that inhibits the function of mutant lamin A G608G, wherein the nucleic acid comprises a nucleic acid sequence as listed in any of SEQ ID NOS:6-48. In certain aspects, the inhibitory RNA molecule comprises the sequence AA(N19)UU wherein N19 represents the sequence of any of SEQ ID NOS:7-21, or a sequence which is 99%, 98%, 97%, 96%, 95%, 94%, 93%, 92%, 91%, 90%, 89%, 88%, 87%, 86%, 85% identical to the sequence of any of SEQ ID NOS:7-21. In certain aspects, the inhibitory RNA molecule comprises the sequence AA(N20)UU wherein N20 represents the sequence of any of SEQ ID NOS:22-35, or a sequence which is 99%, 98%, 97%, 96%, 95%, 94%, 93%, 92%, 91%, 90%, 89%, 88%, 87%, 86%, 85% identical to the sequence of any of SEQ ID NOS:22-35. In certain aspects, the inhibitory RNA molecule comprises the sequence AA(N21)UU wherein N21 represents the sequence of any of SEQ ID NOS:36-48, or a sequence which is 99%, 98%, 97%, 96%, 95%, 94%, 93%, 92%, 91%, 90%, 89%, 88%, 87%, 86%, 85% identical to the sequence of any of SEQ ID NOS:36-48. The invention provides a nucleic acid encoding an inhibitory RNA molecule that inhibits the function of mutant lamin A G608G, wherein the nucleic acid consists essentially of a nucleic acid sequence as listed in any of SEQ ID NOS:6-48.

In other aspects, the invention provides a composition comprising the nucleic acid that encodes an inhibitory RNA molecule that inhibits the function of mutant lamin A G608G. In other aspects, the invention provides a composition comprising a mixture of at least two of the nucleic acid that encodes an inhibitory RNA molecule that inhibits the function of mutant lamin A G608G. In other aspects, the invention provides a composition comprising a mixture of about 3, 5, 10, 15, 20, 25, 30, 35 of any of the nucleic acids that encode an inhibitory RNA molecule that inhibits the function of mutant lamin A G608G. In another aspect, the invention provides n expression vector that comprises a nucleic acid that encodes an inhibitory RNA molecule that inhibits the function of mutant lamin A G608G. In yet another aspect, the invention provides a composition comprising the expression vector that comprises a nucleic acid that encodes an inhibitory RNA molecule that inhibits the function of mutant lamin A G608G.

The invention provides a method for inhibiting the function of mutant lamin A G608G, wherein the method comprises contacting a nucleic acid that encodes an inhibitory RNA molecule that inhibits the function of mutant lamin A G608G to a cell comprising a nucleic acid which encodes mutant lamin A G608G. The invention provides a method for treating a biological condition associated with a lamin A G608G mutation in a subject, the method comprising administering to a subject an effective amount of the nucleic acid that encodes an inhibitory RNA molecule that inhibits the function of mutant lamin A G608G. In other aspects, the invention provides a method for treating a biological condition associated with a lamin A G608G mutation in a subject, the method comprising administering to a subject an effective amount of the composition comprising a nucleic acid that encodes an inhibitory RNA molecule that inhibits the function of mutant lamin A G608G. The invention provides a method for treating a biological condition associated with a lamin A G608G mutation in a subject, the method comprising administering to a subject an effective amount of the expression vector that comprises a nucleic acid that encodes an inhibitory RNA molecule that inhibits the function of mutant lamin A G608G. In certain aspects, the subject is affected with a progeroid syndrome. In certain aspects, the subject is affected with HGPS. Methods for administering nucleic acids, vectors comprising nucleic acids, and compositions comprising such nucleic acids and vectors are known in the art, and are contemplated herein.

The invention provides a method for treating a biological condition associated with a lamin A G608G mutation in a subject, the method comprising administering to a subject an effective amount of an inhibitor of mutant lamin A G608G. In certain aspects, the inhibitor is identified by any of the methods which are described herein. In certain aspects, the inhibitor is a peptide of the invention, a small molecule inhibitor, an antibody, or an antibody fragment. In certain aspects, the biological condition which is being treated by any of the methods of treatment is progeria.

Hutchinson-Gilford progeria syndrome (HGPS, OMIM 176670) is a rare disorder that is characterized by segmental accelerated aging and early death, frequently from coronary artery disease (Brown, 1985). In certain aspects, the invention provides methods to dissect the molecular mechanisms of alterations induced by the mutant lamin A G608G on nuclear functions in HGPS cells. Primary cultures of dermal fibroblasts from subjects carrying the LMNA G608G mutation can be used as a model system. In certain aspect, the temporal and spatial mode of expression of the progerin protein can be monitored. By combining biochemical and morphological analyses, it can be determined how progerin acts as a dominant negative factor on the assembly state of the nuclear lamin network. The effect of progerin accumulation on inner nuclear envelope composition, nuclear pore repartition, chromatin organization and its relationship with known lamin A partners can be examined. Various functional assays known in the art and as described herein can be used to investigate the effect of progerin expression on nuclear transport, heat shock response, and cell cycle progression. In certain aspects the invention provides that mutant lamin A accumulates in the nucleus in an age dependent manner and acts as a dominant negative mutant by altering the nuclear lamin network and inducing nuclear invaginations.

In another aspect, the invention contemplates small interfering RNA which can be useful in methods for attenuating and/or inhibiting progerin expression thereby providing a method for reversing and/or slowing down the HGPS cellular phenotype by with small interfering RNA.

In other aspects, the invention provides methods to identify lamin A-interacting proteins and to characterize which of these lamin A interacting proteins no longer interact with progerin. Progerin destabilizes some tissue-specific lamin A interactions and thereby alters their function. In certain aspects, lamin A and/or progerin interacting proteins can be identified in coimmunoprecipitation assays with anti-lamin A or anti-progerin specific antibodies using nuclear extract isolated from HGPS fibroblasts. In another aspect, a stable endothelial cell line expressing an His6-tagged lamin A or progerin can be used to isolate interacting partners by affinity purification. These approaches can be combined with proteomic analyses to identify interacting partners. The distribution of any such proteins in the nucleus can be determined in the presence of progerin.

In certain aspects, the invention provides methods to determine common cellular and molecular mechanisms underlying atypical and adult progeroid syndromes in comparison to HGPS. Cellular, nuclear and functional alterations identified in HGPS G608G fibroblasts will be tested in other lamin A-associated premature aging disorders, such as the atypical and adult progeroid syndromes. It can be determined whether mechanisms responsible for HGPS as described herein, are responsible for other syndromes of premature ageing. The invention provides methods to determine nuclear changes that elicit premature cellular aging.

Nearly 90% of the subjects affected with HGPS carry LMNA G608G (GGC>GGT), within exon 11 of LMNA activating a splicing donor site which elicits a deletion of 50 amino acids at the carboxyl-terminal of prelamin A (Cao et al, 2003; De Sandre-Giovannoli, 2003; Eriksson, 2003). Eighty skin biopsies from unaffected individuals, age 30 to 97 years old, female and male, were screened. In certain aspects, the invention provides that a splicing event similar to the splicing event constitutively active in HGPS cells, occurs sporadically in unaffected individuals at all age, wherein lamin A G808G polypeptide is detected in some elderly individuals who do not suffer from HGPS. The aberrant spliced mRNA transcript does not increase with age. The invention provides that the mutant lamin A protein (progerin) is detectable in skin biopsy sections derived from elderly individuals. Anti-progerin specific antibody revealed that progerin is detectable in the nuclear compartment of a subset of dermal fibroblasts, dispersed throughout the dermis and only on skin sections exhibiting advanced morphological defects related to aging. Since progerin expression levels remain below detection in young normal skin cells, progerin can accumulate within the nuclei of dermal fibroblasts that may have reached the end of their replicative lifespan and/or as cellular aging develops. In certain aspects, the invention provides that HGPS cells mimic the typical cellular defects induced by age, wherein the cellular defects in HGPS cells are observed more rapidly as a result of the higher levels of progerin mRNA transcript observed in HGPS cells.

In certain aspects, the invention provides that LMNA G608G accumulates in an age-related manner and acts as a dominant negative mutant by altering the lamina scaffold, NE growth, nuclear pore distribution, and/or chromatin distribution. These nuclear alterations lead to inhibition of cell-cycle progression, alteration of cell motility, and early onset of cellular senescence. The presence of LMNA G608G also established an altered response to heat stress, and probably to shear stress. Provides are in vivo cellular and tissue localization analyses of a mutant lamin A responsible for the most devastating laminopathy: Hutchinson-Gilford progeria syndrome. Since LMNA G608G accumulates primarily in vascular cell types, it can be regarded as a key player in the onset of atherosclerosis, primary cause of death for HGPS patients. Preventing and/or attenuating LMNA G608G accumulation, expression, localization and/or posttranslational can provide therapeutic methods that reduce the progression of atherosclerosis.

### BRIEF DESCRIPTION OF THE FIGURES

FIG. 1 shows primary structural elements in Lamin A. LMNA contains 12 exons and are indicated along their corresponding amino acid residues. The rod domain is identified by four coiled-coil domains: 1A, 1B, 2A, and 2B, interrupted by linker that are non helical. There is a six-heptad extension in coil 1B (orange box); yellow box denotes conserved sequence among IFPs. The globular tail domain contains a nuclear localization signal (NLS), an immunoglobulin (Ig) fold, and the CAAX motif.

FIG. 2 shows a schematic representation of some known lamin A interacting partners. Illustrating the complexity of lamin A interactions and its possible functional roles in DNA replication, transcription, chromatin organization, nuclear positioning, nuclear scaffold and shape, as well as in cell division and differentiation. A denotes lamin A, B lamin B, C lamin C, Em emerin, LaP2b and LAp2a for lamin associated polypeptides 2b and 2a respectively, transcription factors: MOK2, SREBP1, Rb (retinoblastoma protein). BAF corresponds to auto integration factor, Narf for nuclear recognition factor, Lcol for lamin companion 1 and PKCa denotes protein kinase C alpha.

FIG. 3 shows cDNA sequence of progerin fragment from nucleotide 1804 to the end the coding region (SEQ ID NO:54), and the corresponding amino acid reading frame (SEQ ID NO:3) starting from residue 602.

FIG. 4 shows that an antibody of the invention specifically detects the mutant lamin A G608G (progerin) in HGPS cells and demonstrates the accumulation of the mutant protein progerin over time. (A) Immunofluorescence microscopy was performed on fibroblasts from an unaffected individual (population doublings 40) and a subject with HGPS (PPDs 24, 30, 42, and 52). Cells were stained with anti-LMNA G608G (red), and anti-emerin (green), merged signals are indicated. Note the increase of positively labeled HGPS with increasing PPDs. (B) Western blot of control (PPDs 38) and HGPS (PPDs: 28, 35 and 45) extracts probed with anti-LMNA G608G and anti-lamin A/C. (C HGPS and control fibroblasts double labeled with anti-LMNA G608G (red) and either anti-lamin A, anti-emerin, or anti-nucleoporin 414 (green) reveal the similar distribution into cable-like structures of mutant and wild type proteins. Chromatin was stained with DAPI (blue). Scale bar, 10 μm.

FIG. 5 shows progerin accumulation induces nuclear envelope invaginations as shown by electron microscopy on ultrathin sections from control (A and B) and HGPS(C to F) cells at PPDs 38. Low magnification of control (A) and HGPS(C) nuclei (bar, 5 μm); (B, D to F) high magnifications of the corresponding nuclei, at the nuclear envelope invaginations (NI); bar, 0.5 μm. NE denotes nuclear envelope, NC nucleoplasm, C cytoplasm, and NPC nuclear pore complex. While few, small nuclear invaginations are detected in control fibroblasts, HGPS nuclei exhibit numerous, large nuclear invaginations recognizable by the presence of high density of clustered nuclear pores.

FIG. 6 shows that FTI-277 treatment prevents nuclear deformations and delocalizes progerin. (A) Nuclei from control and HGPS cells (PPDs 35-38) were scored for abnormal shape every twenty-four hours after daily treatment with 20 μM FTI-277 for three days and compared against scores for untreated cells. Shown are average percentages of cells with abnormally shaped nuclei for each treatment or time course (error bars at one standard deviation). (B) Immunofluorescence microscopy on FTI-277-treated or -untreated cells using anti-LMNA G608G (red), anti-prelamin A (green), and Dapi (blue), demonstrates the gradual relocation of the mutant protein progerin away from the nuclear envelope and lamina and into intranuclear foci. Scale bar, 10 μm.

FIG. 7 shows mutant LMNA G608G accumulation reduces cellular proliferation, induces premature senescence, and impairs cell migration in HGPS fibroblasts. (A) Proliferative index determined by anti-Ki-67 labeling, was shown to decline faster in HGPS cells than in control cells. (B) Senescence β-Gal staining revealed more cells of a 4-week old HGPS culture (55 PPDs) to be senescent versus those of a 1-week-old control culture (55 PPDs). (C) The average percentages of cells scored as senescent β-gal positive from HGPS cells after 1, 4, and 6 weeks (55 PPDs) were significantly higher than 1 and 4 week control cultures (55 PPDs). (D) HGPS cells (55 PPDs) assayed for senescence-associated β-galactosidase (SA-β-gal) also showed a high level of anti-LMNA G608G signal (red); cells were counterstained with Dapi. Bar, 10 μm. (E) Immunofluorescent labeling of control and HGPS cells (38 PPDs) subjected to migration stimulus revealed an impaired ability to migrate among cells with a bright anti-LMNA G608G signal (red); emerin (green). Merged images are indicated.

FIG. 8 shows that mutant lamin A G608G was present mostly in vascular cells on skin sections derived from a subject with HGPS. (A) Three serial skin sections were immunostained with anti-lamin A/C antibody, anti-LMNA G608G antibody, or the corresponding preimmune serum. Low magnification, hematoxylin and eosin staining: epidermis (ep), dermis (de), sweat glands (SG), capillary (C), and arrector pili muscle (apm). Lamin A/C was detected in the nuclei of most cells from the epidermis and in the dermal compartments. Progerin, as detected by anti-LMNA G608G, was restricted to nuclei within the blood vessel (C), some cells surrounding the sweat glands (SG) and the arrector pili muscle (apm). Lower panels correspond to staining with the preimmune serum (PIM) of the rabbit immunized with the LMNA G608G peptide. (B) High magnification of blood vessels from HGPS skin sections immuno-labeled with anti-LMNA G608G and anti-α smooth muscle actin (panel SMA) or anti-CD31 antibodies (panel CD31) and counterstained with Dapi. Triple merged signals are indicated. The mutant lamin A accumulates primarily in the vascular cells: smooth muscle cells and endothelial cells. (C) Normal skin sections immunolabelled with anti-lamin AG608G, anti-lamin A/C and anti-α smooth muscle actin (SMA) antibodies or stained with hematoxylin and eosin. SBG denotes sebaceous glands. Note the presence of A-type lamins in most nuclei from the epidermal and dermal compartments.

FIG. 9 shows a schematic representation of human prelamin A protein and regions recognized by anti-A-type lamin abs used in the study. Prelamin A and lamin A G608G, progerin, and amino acid sequences are shown in parallel. The 50-aa residues truncated in the progerin sequence are indicated. The positions of the peptides used to generate anti-lamin A/C (1), anti-lamin A Jo14 (Serotec, Raleigh, N.C.), and anti-prelamin A C-20 (Santa Cruz Biotechnology) are indicated on the prelamin A sequence. The position and sequence of the peptide used to generate anti-lamin A G608G is indicated on the progerin sequence.

FIG. 10 shows comparison of the lamin A/C intra-nuclear distribution with that of lamin B1, emerin and chromatin in the most dysmorphic nuclei in HGPS patient PT001 fibroblasts. Upper panel: HGPS fibroblasts immunostained with anti-lamin A/C (panel: LmA/C), anti-lamin B (panel: LmB) and DAPI. Lower panel: HGPS fibroblasts immuno-labeled with anti-lamin A/C (panel: LmA/C), anti-emerin (panel emerin) and DAPI (panel DAPI). A-type lamins co-localized with lamin B1 and emerin signals. Bars at 10 μm.

FIG. 11 shows confocal analysis of dermal fibroblast after heat shock. Cells were processed for indirect immuno-fluorescence labeling using anti-lamin A/C, panels A/C or anti-lamin B1 and anti-vimentin, panels B/Vim. Control and HGPS fibroblasts are indicated as C or P respectively. There is increased number of dysmorphic nuclei in HGPS patient cells (P) versus control cells at time point 24 hours of recovery from heat shock. The cytoplasmic intermediate filament network remained unaltered by heat shock both in control and in patient fibroblasts.

FIG. 12 shows that Progerin colocalizes with lamin A network. Double immunofluorescence staining was performed on primary fibroblast cultures derived from unaffected (control) and HGPS affected subjects (HGADFN127 and PT001) at passage number 9. Progerin panels denote staining with anti-progerin. Lamin A panels correspond to anti-lamin A (Jo14). Dapi Panels correspond to the DNA staining. Note the absence of progerin signal in control fibroblasts.

FIG. 13 shows that Progerin is expressed at different levels in HGPS fibroblasts. Double immunofluorescence staining of HGPS fibroblasts and control cells at passage number 11. Progerin panels correspond to the staining with anti-progerin antibody. Lamin B1 panels correspond to the anti-lamin B1 antibody signal. The DNA was stained with Dapi.

FIG. 14 shows that Progerin accumulation in HGPS cells has a dominant negative effect on the lamin filament network and on the chromatin organization. HGPS PT001 and control fibroblasts from passage number P14 were fixed and submitted to indirect immunofluorescence detection with anti-progerin (Progerin panels), anti-lamin A (Jo14, Lamin A panels), and the DNA was stained with Dapi (Dapi panels). Note the reorganization of the lamin network into collapsed, thick cable-like structures and aggregates. The chromatin in HGPS PT1001 is more compact and dense in some areas, and the rearrangement can cause the formation of an intra-nuclear hole (lower Dapi panel). Signals from progerin and lamin A were superimposed in the Merge panels.

FIG. 15 shows a representative Western blot analysis. Total cell extracts from control (lane C) and HGPS HGADFN127 (lane HGPS) from passage number P14 were separated on a 10% SDS-PAGE gel. One replica of the gel was stained with coomassie blue staining solution (Coomassie) and one identical replica was transferred to nitrocellulose and incubated with anti-progerin antibody (anti-progerin panel), the same blot was washed and re-probed with anti-lamin A/C antibody (anti-lamin A/C panel). Note the presence of one single strongly labeled band at approximately 66 kDa in the HGPS lane that corresponds to the progerin protein, no signal is observed in the control nuclei extract (Lane C). The rabbit polyclonal anti-progerin antibody specifically recognizes progerin and does not cross-react with lamin A and C.

FIG. 16 shows in situ localization of progerin expression on skin sections derived from a skin biopsy of a 9 year-old subject with HGPS. Panels H&E correspond to a 6 μm skin section stained with hematoxylin and eosin. Two pictures at low magnification (×10) are shown to illustrate the morphology of the skin: epidermis (ep), dermis (de), sweat glands (SG), capillary (C), and arrector pili muscle (apm). Three serial sections were used; one for H&E staining, one was stained with anti-lamin A/C antibody (panels a to d) and the third with anti-progerin antibody (panels e to h). Structures in the dermis are indicated on the Dapi stained panels to give comparative coordinates to the H&E pictures. Lamin A and C are detected in all nuclei, and that progerin is detected only in a subset of cells: in cells surrounding the sweat glands (panel e) and within the capillary (panel g).

FIG. 17 shows progerin and lamin A protein complexes isolated from HGPS nuclear extracts by coimmunoprecipitation assay. The first lane corresponds to 10% of total nuclear extract used for each assay. Lane C corresponds to proteins isolated with control rabbit IgG. Lane P corresponds to protein complexes isolated with anti-progerin IgG. Lane A corresponds to immunocomplexes isolated with mouse monoclonal anti-lamin A Jo14. (*) indicates the position of some protein bands unique to lamin A and progerin complexes. (**) denotes proteins of different amounts in progerin and lamin A complexes. Arrowheads indicate protein bands submitted for mass spectrometry analysis.

FIG. 18 shows progerin expression in HGPS fibroblasts. Double immunofluorescence staining of HGPS fibroblasts and control cells at passage PPDs 22. Panels: Progerin corresponds to the staining with anti-progerin antibody. Panels: Lamin B1 correspond to the anti-lamin B1 antibody signal and the DNA was stained with Dapi.

FIG. 19 shows immunofluorescence staining of a skin section derived from an 85 year old female. Skin section was probed with anti-progerin and counterstained with Dapi. Note the presence of a few positively labeled nuclei in the dermis indicating the presence of the mutant lamin A G608G.

FIG. 20 shows adult control fibroblasts from PPDs 20, grown to 80% confluency in medium containing 15% fetal serum were transferred for 24 hours to medium without serum. Culture medium was collected and TCA precipitated to allow for recovery of the secreted proteins. 70 μg of the total protein preparation was separated on IEF strip pH 3 to 10 and subsequently separation on a 12% acrylamide gel and stained by silver stain method. Note the presence of several protein spots at pI 3 to 4 and molecular weight 30 to 220 kDa.

FIG. 21 shows a representative western blot analysis of protein preparations from conditioned medium derived from control adult (Cont) and HGPS cultures (HGPS) were probed with anti-IGFBP-3 antibody. Note the low amount of IGFBP-3 present after 24 hours culture in medium without serum (HGPS-24). The level of IGFBP-3 in HGPS cells (HGPS-24b) dramatically increased in medium collected after a second round of serum starvation.

FIG. 22 shows progerin expression in human skin. FIG. 22A. RT-PCR analysis of HGPS cells and human skin biopsies of indicated age, primers amplifying wild-type and progerin transcripts. FIG. 22B. Direct sequencing of a short portion within exon 11 of wild type lamin A (LMNA), and progerin transcripts from HGPS and 93-year-old subjects. FIG. 22C. Western blot analysis of protein extracted from skin biopsies of indicated age with anti-progerin 972S9, anti-lamin A/C and anti-actin antibodies.

FIG. 23 shows progerin expression in primary dermal fibroblast cultures. FIG. 23A. Immunofluorescence microscopy on primary dermal fibroblasts from an HGPS subject and unaffected individuals of indicated ages with rabbit monoclonal anti-progerin antibody. FIG. 23B. Immunofluoresence detection of progerin in HGADFN 127 (HGPS individual) and DR118 (86-year-old individual) at late PPDs. FIG. 23C. Western blot analysis of nuclear protein extracts derived from fibroblast cultures HGADFN 127 (HGPS) at PPD 20 and DR118 (86-year-old female) at PPD 15 and 30, respectively, were immunoprecipitated with anti-progerin mAb 972S9 and the corresponding Western blot was probed with the same antibody.

FIG. 24 shows in situ localization of progerin on human skin sections derived from a subject with HGPS and from unaffected individuals. FIG. 24A. HGPS skin sections immunostained with anti-progerin (prog), anti-lamin A (LMNA) antibody, or anti-α smooth muscle actin antibody (αSMA) and counterstained with a DNA stain (dapi). Morphologic entities are indicated: epidermis (ep), dermis (de), sweat glands (SG), capillary (c), arrector pili muscle (a), and hair follicle (h). FIG. 24B. Newborn foreskin sections immunolabelled with anti-progerin or anti-αSMA antibody. FIG. 24C. Breast skin sections from 22- and 46-year-old female subjects probed with anti-progerin and lamin A. The respective double or triple merged signals are indicated.

FIG. 25 shows progerin accumulation in human elderly skin biopsy sections. FIG. 25A. Forehead skin section from a 69-year-old individual probed with anti-progerin antibody and counterstained with dapi. Bars correspond to 100 and 50 μm, respectively. FIG. 25B. Forehead skin section from a 93-year-old donor. FIG. 25C. Skin sections from different body sites as indicated were probed with anti-progerin and anti-lamin A (LMNA) antibodies. Bar, 50 μm. Merged images are indicated.

FIG. 26 shows progerin detection in a subset of terminally differentiated keratinocytes. Left panels correspond to anti-progerin monoclonal antibody staining of skin sections derived from individuals of indicated age. Right panels correspond to the merged signal of dapi and progerin signals. Square indicates the zoomed in region of the epidermis. ep denotes epidermis, and de the dermis.

### DETAILED DESCRIPTION

The peptides of the invention are isolated peptides. As used herein, the term “isolated peptides” means that the peptides are substantially pure and are essentially free of other substances with which they may be found in nature or in vivo systems to an extent practical and appropriate for their intended use. In particular, the peptides are sufficiently pure and are sufficiently free from other biological constituents of their hosts cells so as to be useful in, for example, producing pharmaceutical preparations or sequencing. Because an isolated peptide of the invention may be admixed with a pharmaceutically acceptable carrier in a pharmaceutical preparation, the peptide may comprise only a small percentage by weight of the preparation. The peptide is nonetheless substantially pure in that it has been substantially separated from the substances with which it may be associated in living systems, or during in vitro peptide synthesis.

The term “therapeutically effective amount” used interchangeably with the term “effective amount” as used herein means that amount of a compound, material, and/or an agent, such as the peptides of the present invention, or composition comprising a compound of the present invention which is effective for producing a therapeutic effect by modulating mutant lamin A function, localization and/or expression levels in at least a sub-population of cells in a subject, and thereby modulating mutant lamin A in a subject at a reasonable benefit/risk ratio applicable to any medical treatment.

The terms “treatment” or “treat” as used herein include treating, preventing, ameliorating, and/or decreasing the severity of the symptoms of a disease or disorder, or improving prognosis for recovery.

The term “mutant lamin A” refers to lamin A polypeptides which have alterations, such but not limited to insertions, deletions, substitutions, in the polypeptide sequence such that the altered polypeptide is functionally different compared to the normal mutant lamin A polypeptide.

Hutchinson-Gilford progeria syndrome (HGPS, OMIM 176670) is a rare disorder that is characterized by segmental premature aging and death between 7 and 20 years of age from severe premature atherosclerosis. Mutations in the LMNA gene are responsible for this syndrome. Approximately 80% of HGPS cases are caused by a G608G (GGC>GGT) mutation within exon 11 of LMNA, which elicits a deletion of 50 amino acids near the carboxyl-terminal of prelamin A. The invention provides that the mutant lamin A (progerin) accumulates in the nucleus in a cellular age-dependent manner. In human HGPS fibroblast cultures, severe nuclear envelope deformations and invaginations were observed, concomitantly to nuclear progerin accumulation. Nuclear envelope deformations and invaginations were preventable by farnesyltransferase inhibition. Nuclear alterations affect cell-cycle progression and cell migration and elicit premature senescence. Skin biopsy sections from a subject with HGPS showed that the truncated lamin A accumulates primarily in the nuclei of vascular cells. This is the first evidence that accumulation of progerin is directly involved in vascular disease in Progeria.

The LMNA G608G mutation in Hutchinson-Gilford Progeria Syndrome (HGPS) produces a truncated protein lacking amino acids 607 to 656 of prelamin A but retaining the CAAX box, a target for the prenylation, at its carboxyl-terminus. A-type and B-type lamins are members of the intermediate filament protein superfamily and are the building blocks of the nuclear lamina. B-type lamins are permanently isoprenylated whereas lamin A loses its modification by lamin A-specific processing steps involving Zmpste24 endoprotease after incorporation into the lamina. Because the endoproteolytic cleavage site is lost in the truncated lamin A (progerin) it is permanently prenylated.

Previous studies of A-type lamin distribution in primary dermal fibroblasts from subjects with HGPS showed nuclear abnormalities in size and shape in a subpopulation of cells in culture. Similar alterations were also reported in different syndromes caused by other mutations of the LMNA gene. The lack of specific tools to directly visualize mutant lamin proteins has so far been a limiting factor in addressing the pathogenesis of laminopathies. The new reading frame in the carboxyl-terminal domain of the truncated HGPS lamin A allowed generation of a specific anti-LMNA G608G antibody.

Lamin A and C Proteins

The nuclear lamins (A and B-type) are members of the intermediate filament proteins (IFPs) superfamily (Fuchs and Weber, 1994). The primary sequence of the lamins differs from that of the cytoplasmic IFPs. All IFPs are organized around a central rod domain that comprises four coiled-coil domains, which are separated by flexible linker regions, and a globular head and tail domain. Sequence comparison between the nuclear lamin proteins with the cytoplasmic IFs reveals an extra 42 amino acid (six heptads) in coil 1B (Fisher et al., 1986; Riemer et al., 1998). The nuclear lamins harbor two unique sequences; one consists of a nuclear-localization signal (NLS) sequence in the tail domain responsible for the targeting to the nuclear compartment (Frangioni and Neel, 1993), and the second consists of a carboxyl-terminal CAAX box (cysteine-aliphatic-aliphatic-any amino acid), which is a target for isoprenylation (Holt et al., 2001; Krohne et al., 1989; Vorburger et al., 1989). The precursors of lamin A, B1, and B2 have a CAAX motif at their carboxyl-terminus and undergo posttranslational modifications and prenylation. In contrast, lamin C an alternative splice variant of lamin A lacks the CAAX motif and therefore is not prenylated (Lin and Worman, 1993). The conversion of pre-lamin A to lamin A involves a first step of farnesylation of the CAAX box, followed by proteolytic removal of the three carboxyl-terminal residues AAX, then by the carboxyl-methylation of the terminal cysteine, ending with an endoproteolysis step that gives rise to the mature lamin A which lacks the last 15 carboxyl-terminal amino acids upstream from the cysteine residue (Gutierrez et al., 1989).

The mutation G608G of HGPS patients occurs within exon 11 of the LMNA gene. This mutation creates a cryptic splice site in the mutant prelamin A mRNA, producing a 150 nucleotide deletion that results in the deletion of 50 amino acids (608-657) near the carboxyl-terminus of prelamin A (Eriksson et al., 2003; as described herein). The deleted 50 amino acids contain the endoproteolytic cleavage site at residue 646 that is required for the proper synthesis of mature lamin A. Therefore, the resulting truncated lamin A (progerin) is abnormally processed. A detailed study on how the mutant lamin A is processed in HGPS cells is required to identify whether or not the mutant lamin A is prenylated, and if so, at which step of modifications the mutant lamin A is not further processed. Interestingly, the protein Narf binds specifically to farnesylated prelamin A precursors (Barton and Worman, 1999); whether Narf can still bind to progerin remains to be established. Progerin is also missing serine 652 and 657, two potential cyclin-dependent kinase sites that may be important in cell-cycle dependent regulation of lamin assembly status. Their loss in progerin may have a significant impact on assembly and disassembly of A-type lamins during mitosis and therefore will need to be investigated.

Wild-type lamin A interacts with the NE components and nucleoplasmic elements and serves as an important structural scaffold for anchoring components of the different subnuclear compartments. Normal prelamin A processing involves farnesylation and carboxyl-methylation of the cysteine residue within the CAAX motif, followed by a proteolytic cleavage of 15 amino acid residues from the carboxyl-terminus to generate the mature lamin A. Since the last cleavage step cannot occur in LMNA G608G, the truncated lamin A is permanently modified, retaining the hydrophobic modification on the last cysteine residue. This modification can be responsible for stabilizing LMNA G608G interaction with the inner nuclear membrane and thereby induce NE invaginations. Isoprenylation and carboxyl-methylation of CAAX-containing proteins targeted to the nucleus was previously reported to induce intranuclear membrane formation. Analogous to these findings, LMNA G608G would be forcing the inward growth of the NE to provide additional surface area to accommodate the excess LMNA G608G accumulating in HGPS cells.

Lamin A/C Interacting Proteins

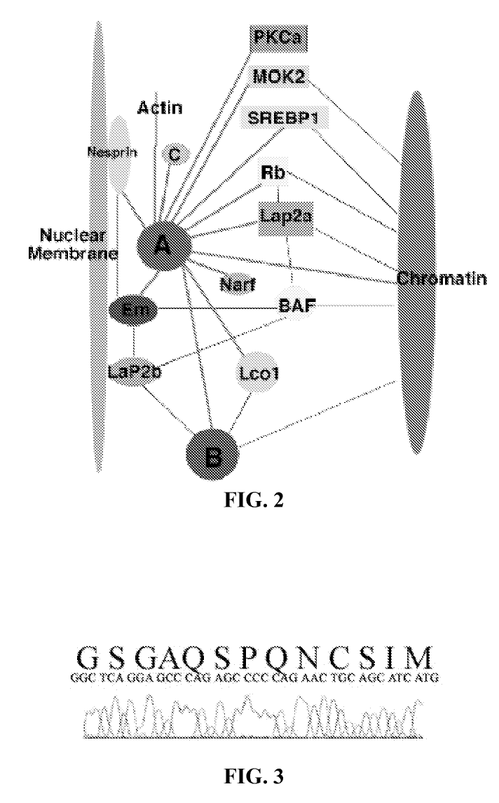

Several lamin-associated proteins have been identified (Zatrow M. S. et al., 2004). Lamin A may serve as a structural scaffold for anchoring or tethering components from different subnuclear compartments as summarized above (FIG. 2). Indeed, lamin A interacts with other lamins (A-type and B-type), integral membrane proteins such as emerin, lamina associated polypeptides (LAP), Nesprin 1a (Foisner and Gerace, 1993; Mislow et al., 2002; Ostlund et al., 1999), and probably other recently identified membrane components: Nurim, LUMA, RFPB, UNC-83, Syne1, Syne2, and Nuance (Burke and Stewart, 2002; Schirmer et al., 2003). Therefore, lamin A interacts directly with nuclear envelope proteins and may play a role in maintaining the integrity of the nuclear envelope. The A-type lamins are associated with splicing factors, as evidenced by their colocalizing with nuclear bodies that are discrete nuclear sites for RNA processing (Jagatheesan et al., 1999). Lamin A is localized throughout the nucleoplasm (Dechat et al., 2000), and may influence gene expression at transcriptional level. Lamin A interacts with the transcription factor retinoblastoma protein (Rb) (Ozaki et al., 1994), suggesting a possible role in Rb pathway, such as regulation of certain sets of genes associated with cell cycle control (Frolov and Dyson, 2004). Lamin A also interacts with the transcription factors SREBP1a and SREBP1b (Lloyd D. J. et al., 2002), which control a set of genes involved in cholesterol biosynthesis and lipogenesis and promote adipocyte differentiation (Horton, 2002). Furthermore, lamin A binds to MOK2, a transcriptional repressor of the cone rod homeobox protein (Crx) (Dreuillet et al., 2002). Moreover, emerin and LAP proteins belong to the LEM-domain family (Lin et al., 2000) and contain a 43 amino acids sequence (the LEM-domain) responsible for their interaction with the barrier to auto-integration factor (BAF) a chromatin-associated protein (Foisner and Gerace, 1993; Lee et al., 2001; Ostlund et al., 1999). These data suggest an important role of A-type lamins in tethering transcription factor complexes, repressor complexes, and chromatin remodeling complexes. In addition, lamin A binds directly but non-specifically to DNA trough its Ig-fold domain (Stierle et al., 2003). As lamin A interacts directly and indirectly with chromatin, it may play an important role in chromatin organization. Lastly, A-type lamins also interact with signaling molecules, such as protein kinase Ca (Martelli et al., 2000), and 12 S-lipoxygenase (Tang et al., 2000). Most of the lamin A-associated proteins have been identified by in vitro assays and the list continues to grow. The best interactions characterized so far are with LAP1a and emerin. Functional analysis of each of these interactions listed above will be required to clearly define lamin A physiological implication(s) and several of them will be addressed by methods described herein.

Progerin Effects at the Cellular Level

HGPS cells in culture display several nuclear abnormalities in size and shape (as described herein; Eriksson et al., 2003; Goldman et al., 2004). A recent study showed that HGPS cells growth rate exhibits an initial period of hyper proliferation that terminates with an increased rate of apoptosis and a premature accumulation of senescent cells (Bridger and Kill, 2004). The finding that telomere lengths are reduced in HGPS cells supports the notion of early onset of senescence in HGPS cells (Allsopp et al., 1996). One study addressed the role of the mutant lamin A, progerin, on the nuclear structural changes observed in HGPS cells (Goldman et al., 2004). Several important observations emerged from this study: first, the number of abnormal nuclei increases with the cellular passage numbers; second, the thickness of the lamina increases coincidentally with the nuclear shape changes; third, in late passage number (24 to 26), lamin B1 and nucleoporin Nup 153 are mislocalized, and there is a loss of peripheral heterochromatin (Goldman et al., 2004). Notably, the mutant lamin A was detected by Western blot analysis only in late passages, indicating that in early cell passages the level of progerin expression must be below detection by this method. A quantitative analysis of the lamin A mRNA content in HGPS cells showed that the abnormally spliced mRNA encoding progerin constituted the majority (84.5%) of the total steady state mRNA derived from the mutant allele (Reddel C. J. and Weiss, 2004). Progerin transcripts comprised 40% of all the lamin A transcripts obtained from both alleles. Microinjection of bacterially expressed progerin or transfection of a vector containing progerin cDNA into HeLa cells recapitulates similar nuclear abnormalities as the one observed in HGPS fibroblasts (Goldman et al., 2004). These results indicate that progerin is directly responsible for those nuclear alterations and as such acts a dominant negative mutant.

Lamin A Mutation in Atypic Progeroid Disorders.

Several LMNA mutations causing HGPS or Atypic Progeroid Syndromes have been reported so far and are summarized in Table 1. Four different mutations in LMNA result in the activation of a new cryptic splice site that causes the production of a truncated mRNA (Table 1). In HGPS, the mutation typically results in the deletion of the 50 amino acids at the carboxyl tail domain of lamin A. In one severe case of restrictive dermopathy, the patient died at the age of six months from respiratory distress; in that case the LMNA mutation caused the deletion of the complete exon 11, corresponding to a 90 residue deletion at the tail of lamin A (Navarro et al., 2004). In one case of atypical adult HGPS, the LMNA mutation resulted in 35 residues deletion in the same region (Fukuchi et al., 2004). In all the above mutants, the truncated regions overlap within the carboxyl-terminal domain of lamin A, and they retain a CAAX-box motif for farnesylation but lack an endoproteolytic cleavage site. Another atypical progeria case with a milder phenotype harbors a LMNA R644C (Csoka et al., 2004); this heterozygous missense mutation is located in the cleavage recognition site for the prelamin A endoprotease zmpste24 (Bergo et al., 2002; Leung et al., 2001). Based on the phenotypic severities of those cases, it appears that unprocessed prelamin A has a deleterious effect on lamin A function and that the truncated forms of lamin A have dominant negative effects that are proportionally related to the extent of the truncation within the carboxyl-tail domain of lamin A. Potential common molecular mechanisms of those mutant lamin A proteins can be investigated by the methods described herein.

TABLE 1
Summary of LMNA mutations in HGPS and atypical Progeroid syndromes

| Exon | Mutation | Effect | No of subjects | Reference |
|---|---|---|---|---|
| 11 | 1824 C > T | G608G | 22 (heterozygote) | Eriksson et al., 2003; De |
| | | Cryptic splice site (50-AA del) | | Sandre-Giovannoli et al., 2003; |
| | | | | Cao and Hegele, 2003; Navarro |
| | | | | et al., 2004; described herein |
| 11 | 1822 G > A | G608S | 1 (heterozygote) | Eriksson et al., 2003; Cao and |
| | | Cryptic splice site (50-AA del) | | Hegele, 2003 |
| 11 | 1868 C > G | T623S | 1 (heterozygote) | Fukuchi et al., 2004 |
| | | Cryptic splice site (35-AA del) | | |
| 11 | IVS11 + 1G > A | Cryptic splice site (90-AA del) | 1 (heterozygote) | Navarro et al., 2004 |
| 11 | 1930 C > T | R644C | 1 (heterozygote) | Csoka et al., 2004 |
| 11 | 1733 A > T | E578V | 1 (heterozygote) | |
| 1 | 29 C > T | T10I | 1 (heterozygote) | |
| 10 | 1626 G > C | K542N | 4 (homozygote) | Plasilova et al., 2004 |
| 8, 9 | 1623 C > T | R471C | 1 (compound | Cao and Hegele, 2003 |
| | 1791 C > T | R527C | heterozygote) | |
| 2 | 433 G > A | E145K | 1 (heterozygote) | Eriksson et al., 2003 |

Hutchinson-Gilford progeria syndrome (HGPS) is a rare fatal genetic disorder that is characterized by accelerated ageing in children. The LMNA gene encoding the A-type lamins A and C is the causative gene of HGPS (De Sandre-Giovannoli et al, 2003; Cao et al, 2003; Eriksson et al, 2003). Approximately 80% of HGPS cases carry the heterozygous silent point mutation G608G within exon 11 of LMNA (Eriksson et al, 2003). This mutation creates an abnormal splice donor site, which produces a truncated protein (progerin) lacking residues 607-656 of prelamin A but retaining the C-terminal CAAX box, a target for prenylation ((De Sandre-Giovannoli et al, 2003; Cao et al, 2003; Eriksson et al, 2003).

The nuclear lamina is a scaffold, which provides structural and mechanical stability for the nuclear envelope; it consists primarily of type V intermediate filament proteins (A- and B-type lamins) and many inner-nuclear membrane proteins (Fuchs et al, 1994; Steinert et al, 1985; Aebi et al, 1986). Lamins interact with heterochromatin and transcriptional regulators, suggesting their important role in the maintenance of chromatin organization and gene expression (Gruenbaum et al, 2003).

At the nuclear envelope periphery, lamin precursors undergo a series of posttranslational modifications. B-type lamins are permanently isoprenylated, whereas prelamin A loses its modification after incorporation into the lamina by lamin A-specific processing steps involving Zmpste24 endoprotease (Pendas et al, 2002; Holt et al, 2001; Krohne et al, 1989). Because the endoproteolytic cleavage site is lost in the truncated lamin A (progerin), it was predicted to be permanently prenylated (Eriksson et al, 2003). Direct and indirect analyses have recently confirmed that progerin retains the farnesyl group (Mallampalli et al, 2005, Capell et al, 2005; Glynn et al, 2005; Yang et al, 2005).

Previous studies of A-type lamin distribution in primary dermal fibroblasts from HGPS patients showed nuclear abnormalities in size and shape in a subpopulation of cells in culture (Eriksson et al, 2003; Goldman et al, 2004; Paradisi et al, 2005). Farnesylated progerin appears to be responsible for the nuclear deformations, since administration of farnesyltransferase inhibitors to the HGPS fibroblast cultures normalized the nuclear shape for the majority of the cells in vitro (Mallampalli et al, 2005, Capell et al, 2005; Glynn et al, 2005; Yang et al, 2005).

The lack of specific tools to directly visualize lamin mutants has so far limited investigating the pathogenesis of laminopathies. The new reading frame of the C-terminal domain of the truncated HGPS lamin A allowed the creation of a specific anti-LMNA G608G antibody. The present invention provides a peptide of SEQ ID NO:1. The invention also provides an antibody that specifically recognizes progerin. Immunohistochemistry on biopsied skin sections from an HGPS patient showed that progerin accumulates primarily in the vasculature system; this provided a direct link to the disease pathology. Studies on primary dermal fibroblasts from HGPS indicate that progerin accumulates in the nucleus progressively with cellular age. Concomitant to progerin build up in the nuclear lamina, several cellular changes were induced: increased nuclear envelope invaginations, rapidly decreased growth-rate, premature entry into senescence and impaired migration potency. These functional changes in HGPS fibroblasts provide information about vascular cellular dysfunctions responsible for the progression of atherosclerosis in HGPS subjects.

Hutchinson-Gilford progeria syndrome (HGPS) is a rare disorder that is characterized by accelerated aging and early death, frequently from coronary artery disease. Patients with HGPS present very characteristic features: short stature, alopecia, sculpted nose, prominent scalp veins, and loss of subcutaneous fat, and prominent joints. Mutations in the LMNA gene are responsible for this syndrome; as such, HGPS belongs to the super-family of laminopathies. The common HGPS mutation corresponds to a de novo single-base pair substitution, G608G (GGC>GGT), within exon 11 of LMNA gene. This mutation results in the deletion of 50 amino acids at the carboxyl-terminal tail of prelamin A; the truncated protein is called progerin. The lamins, lamin A in particular, are nuclear structural proteins that bind many important cellular regulators and may play a role in regulating the tissue specific pattern of alterations, involving primary tissues with renewal potency: muscle, skin, and bone.

Mutation in an Italian Kindred with Hutchinson-Gilford Progeria Disease

Described herein is a mutation in a previously uncharacterized patient from Italy. The patient was patient was found to carry the LMNA gene G608G mutation (patient PT001). The patient was first examined at the age of 2 with the diagnosis “stiff skin syndrome”. She was hospitalized in the following month, blood samples were collected, a skin biopsy from the right leg was performed for histological examination, and a dermal fibroblast culture was established from this biopsy sample by explant culture. Fibroblast cultures from this patient will be referred as PT001 cells. To confirm the clinical diagnosis of HGPS, the LMNA gene was sequenced and a heterozygous C to T mutation at nucleotide 1824 in exon 11 was found, corresponding to the known silent point mutation at codon 608, G608G (GGC>GGT) (De Sandre-Giovannoli et al., 2003; Eriksson et al., 2003). Total RNA fraction from PT001 fibroblast cultures were isolated and the cDNA fragment corresponding to nucleotide 1561 to 2010 of the LMNA coding sequence by RT-PCR was amplified using specific primers. FIG. 3 shows the sequencing data for progerin from nucleotide 1804 (corresponding to amino acid 602) to the last codon, and reads as follows: GSGAQSPQNCSIM (SEQ ID NO:3). The progerin sequence from this subject matched the sequence reported for other HGPS patients carrying the G608G mutation (De Sandre-Giovannoli et al., 2003).

Peptides and Nucleic Acids of the Invention:

A peptide having amino acids sequence GAQSPQNC is referred to as SEQ ID NO:1. A peptide having amino acids sequence SGSGAQSPQNC is referred to as SEQ ID NO:49. A peptide having amino acids sequence SGSGAQSPQNCSIM is referred to as SEQ ID NO:50. Nucleic acid having the following nucleotides 5′-GGA GCC CAG AGC CCC CAG AAC TGC AGC ATC ATG is referred to as SEQ ID NO:6.

In one aspect, the invention is directed to an isolated peptide with the amino acid sequence GAQSPQNC (SEQ ID NO:1). In one aspect, the invention is directed to an isolated peptide with the amino acid sequence SGSGAQSPQNCSIM (SEQ ID NO:50). In one aspect, the invention is directed to an isolated peptide with the amino acid sequence SGSGAQSPQNC (SEQ ID NO:49). In certain embodiments, the peptides can be derived from the Lamin A mutant protein G608G. In other aspects, the peptides can be synthesized. A mutation in the LMNA gene, which encodes Lamin A causes Hutchinson-Gilford Progeria Syndrome (HGPS). HGPS is a syndrome of premature aging in young children. G608G is a mutation which results from a nucleotide change that reveals a cryptic splice site in exon 11 of Lamin A. As a result, the G608G protein variant has a 50-aa internal deletion in the C-terminal end of Lamin A. The G608G variant is presumed to act as a dominant negative mutant. A subject with HGPS can provide a model system to study normal aging and more importantly atherosclerosis.

In another aspect, the invention provides that the isolated peptide of SEQ ID NOS: 1, 2, or 3, or a combination thereof can be used agents, which are inhibitors that attenuate, modulate and/or affect the function, localization and/or levels of mutant lamin A G608G. It is possible that treatment of a cell with the peptides of SEQ ID NOS: 1, 2, or 3, or a combination thereof, can function in a dominant negative manner to outcompete mutant lamin A G608G from its site and/or protein complex of interaction. In one aspect, a dominant negative molecule interferes with, and/or inhibits the function or activity of the wild-type protein in a cell. In one aspect, a dominant negative peptide can be an inactive and/or truncated variant of a polypeptide, which, by interacting with the cellular machinery, and/or other components of a (multi)protein complex, displaces an active polypeptide from its interaction with the cellular machinery, and/or a multiprotein complex, and/or competes with the active polypeptide, thereby reducing the effect of the active polypeptide. For example, a dominant negative peptide, which is a truncated version of the polypeptide, may retain interaction with certain binding partners, while the truncated portion may lead to loss of interaction with other binding partners, and/or loss of a protein function.

The concept of dominant negative constructs which inhibit activity of a target, and/or the pathway in which the target functions is well known in the art. In certain aspects, dominant negative constructs can be generated by administration, expression or overexpression of protein fragments, for example but not limited to specific domains that may interact with binding partners, derived from the target protein. In other aspects, dominant negative constructs against a target can be generated by expression of protein fragments and/or variants which have altered binding and/or interaction with proteins in the same functional pathway, which may directly interact with the target. Mutagenesis and screening methods to identify such fragments and/or variants are well known in the art. In other aspects, dominant negative forms can be any other construct and/or fragment which can interfere with the function of the target protein.

The peptides can contain amino acids with charged side chains, such as acidic and basic amino acids. In addition, these peptides may contain one or more D-amino acid residues in place of one or more L-amino acid residues provided that the incorporation of the one or more D-amino acids does not abolish all or so much of the activity of the peptide that it cannot be used in the compositions and methods of the invention. Incorporating D-amino acids in place of L-amino acids is favorable as it may provide additional stability to a peptide.

Chemically synthesized peptides carry free termini thus being electrically charged. In one embodiment, the peptide of the invention is capped at the amino or carboxy terminus, or both termini. Modification of the N- and/or C-terminus can lead to increased stability, increased permeability in cells, and/or increased activity. Examples of amino terminal capping group include but are not limited to a lipoic acid moiety, which can be attached by an amide linkage to the amino terminus of a peptide. Another example of an amino terminal capping group useful in the peptides described herein is an acyl group, which can be attached in an amide linkage to the alpha-amino group of the amino terminal amino acid residue of a peptide.

In addition, in certain cases the amino terminal capping group may be a lysine residue or a polylysine peptide, where the polylysine peptide consists of two, three, or four lysine residues, which can prevent cyclization, crosslinking, or polymerization of the peptide compound. Alternatively, longer polylysine peptides may also be used. Another amino capping group that may be used in the peptides described in the invention is an arginine residue or a polyarginine peptide, where the polyarglnine peptide consists of two, three, or four arginine residues, although longer polyarginine peptides may also be used. Alternatively the peptide compounds described herein may also be a peptide containing both lysine and arginine, where the lysine and arginine containing peptide is two, three, or four residue combinations of the two amino acids in any order, although longer peptides that contain lysine and arginine may also be used. Lysine and arginine containing peptides used as amino terminal capping groups in the peptide compounds described herein may be conveniently incorporated into whatever process is used to synthesize the peptide compounds to yield the derived peptide compound containing the amino terminal capping group.

In another embodiment of the invention, the peptides may contain a carboxy terminal capping group. The primary purpose of this group is to prevent intramolecular cyclization or inactivating intermolecular crosslinking or polymerization. Furthermore, a carboxy terminal capping group may provide additional benefits to the peptide, such as enhanced efficacy, reduced side effects, enhanced antioxidative activity, and/or other desirable biochemical properties. An example of such a useful carboxy terminal capping group is a primary or secondary amine in an amide linkage to the carboxy terminal amino acid residue. Such amines may be added to the Q-carboxyl group of the carboxy terminal amino acid of the peptide using standard amidation chemistry. In another aspect of the invention, the peptide can be modified by any known modification known to one of ordinary skill in the art. In certain aspects, the peptides may be used as peptidomimetics.

In one aspect, the peptide of the invention can be pegylated. Pegylation, can delay the elimination of the peptides from the circulation by a variety of mechanisms. Pegylation inhibits degradation by proteolytic enzymes and, by increasing the apparent molecular size, reduces the rate of renal filtration. Accordingly, PEG-based modifications are useful to prolong circulation time and bioavailability of the peptides. In one embodiment, the peptide of the invention is pegylated with linear PEG molecules. In another embodiment, the peptide is pegylated with branched PEG molecules. The invention further provides amino-, carboxy- and side-chain pegylated peptides. The PEG moiety can be a PEG molecule with a molecular weight greater than 5 kDa. For example the molecular weight can be between 5 kDa and 100 kDa (e.g., 5, 10, 15, 20, 25, 30, 35, 40, 50, 55, 60, 65, 70, 75, 80, 85, 90, 95 or 100 kDa), and more preferably a molecular weight of between 10 kDa and 50 kDa (e.g., 10, 12, 14, 16, 18, 20, 22, 24, 26, 28, 30, 32, 34, 36, 38, 40, 42, 44, 46, 48, 50 kDa). Methods for synthesis of pegylated peptides are well known in the art.

The invention further provides a peptide with a detectable marker attached thereto. In one embodiment, the detectable marker is attached at the C-terminus of the peptide. In another embodiment, the detectable label is attached to the N-terminus. A detectable marker can be a chemical label such as but no limited to radioactive isotopes, fluorescent groups, chemiluminescent label, colorimetric label, an enzymatic marker, and affinity moieties such as biotin that facilitate detection of the labeled peptide. The invention also provides dye-labeled peptides such as but not limited to fluoresceins, rhodamine conjugates. Other chemical labels and methods for attaching chemical labels to peptides are well-known in the art.

Modifications and substitutions are not limited to replacement of amino acids. One skilled in the art will recognize the need to introduce by means of deletion, replacement, or addition other modifications that provide a peptide with of the invention. Examples of such other modifications include incorporation of rare amino acids, dextra (D)-amino acids, glycosylation sites, cytosine for specific disulfide bridge formation. The modified peptides can be chemically synthesized by methods known in the art, or the isolated nucleic acid sequence can be expressed, after site-directed mutagenesis if necessary, in bacteria, yeast, baculovirus, tissue culture and so on.

In one aspect the invention provides a composition which comprises an inventive peptide, as a free peptide or a peptide coupled to a carrier molecule. The peptide may also be used as a conjugate of at least one peptide or a peptide fragment bound to a carrier. The carrier can provide solid phase support for the peptide of the invention. The carrier may be a biological carrier such as a glycosaminoglycan, a proteoglycan, or albumin, or it may be a synthetic polymer such as a polyalkyleneglycol or a synthetic chromatography support. Other carriers include ovalbumin and human serum albumin, other proteins, and polyethylene glycol.

Still other carriers that may be used in the pharmaceutical compositions of this invention include ion exchangers, alumina, aluminum stearate, lecithin, non-albumin serum proteins, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, and wool fat. Such modifications may both increase the apparent affinity and change the stability of a peptide. Although the number of peptide fragments bound to each carrier can vary, typically about 4 to 8 peptide fragments per carrier molecule are bound under standard coupling conditions.

In another aspect of the invention, peptidomimetic compounds, may be designed based upon the amino acid sequences of the peptides of the invention. In one aspect, the peptidomimetic compounds comprise synthetic compounds with conformation substantially similar to the conformation of the peptides of the invention. The structure of the peptidomimetic compound can be similar to the secondary or tertiary structure of the peptide. The structural similarity of the peptidomimetic compound to the secondary or tertiary structure of the inventive peptide provides the peptidomimetic compound with the ability to function in a manner qualitatively identical to activity of the inventive peptide or the peptide fragment from which the peptidomimetic was derived. Furthermore, the peptidomimetic compounds can have additional characteristics that enhance their therapeutic utility, such as increased cell permeability and a prolonged biological half-life.

The backbone of the peptidomimetics are partially or completely non-peptide, but their side groups are identical to the side groups of the amino acid residues that occur in the peptide on which the peptidomimetics are based. Several types of chemical bonds, e.g., ester, thioester, thioamide, retroamide, reduced carbonyl, dimethylene and ketomethylene bonds, are known in the art to be useful substitutes for peptide bonds in the construction of protease-resistant peptidomimetics.

The peptides in the current invention can be synthesized using standard methods known in the art. Direct synthesis of the peptides of the invention may be accomplished using solid-phase peptide synthesis, solution-phase synthesis or other conventional means. For example, in solid-phase synthesis, a suitably protected amino acid residue is attached through its carboxyl group to an insoluble polymeric support, such as a cross-linked polystyrene or polyamide resin. A protected amino acid refers to the presence of protecting groups on both the amino group of the amino acid, as well as on any side chain functional groups. The benefit of side chain protecting groups are that they are stable to the solvents, reagents, and reaction conditions used throughout the synthesis and are removable without affecting the final peptide product. Typically, stepwise synthesis of the polypeptide is carried out by the removal of the N-protecting group from the initial carboxy terminal and coupling it to the next amino acid in the sequence of the polypeptide. The carboxyl group of the incoming amino acid can be activated to react with the N-terminus of the bound amino acid by formation into a reactive group such as formation into a carbodiimide, a symmetric acid anhydride, or an active ester group such as hydroxybenzotriazole or pentafluorophenyl esters. The solid-phase peptide synthesis methods include both the BOC and FMOC methods, which utilizes tert-butyloxycarbonyl, and 9-fluorenylmethloxycarbonyl as the α-amino protecting groups, respectively, both well-known by those of skill in the art (Sambrook et al., Molecular Cloning: A Laboratory Manual, 2nd edition, Cold Spring Harbor Press, Cold Spring Harbor, N.Y.; Ausubel et al., Current Protocols in Molecular Biology, John Wiley and Sons, New York, 1995).

In another embodiment of the invention, the peptides may also be prepared and stored in a salt form. Various salt forms of the peptides may also be formed or interchanged by any of the various methods known in the art, e.g., by using various ion exchange chromatography methods. Cationic counter ions that may be used in the compositions include, but are not limited to, amines, such as ammonium ions, metal ions, especially monovalent, divalent, or trivalent ions of alkali metals including sodium, potassium, lithium, cesium; alkaline earth metals including calcium, magnesium, barium; transition metals such as iron, manganese, zinc, cadmium, molybdenum; other metals like aluminum; and possible combinations of these. Anionic counter ions that may be used in the compositions described herein include chloride, fluoride, acetate, trifluoroacetate, phosphate, sulfate, carbonate, citrate, ascorbate, sorbate, glutarate, ketoglutarate, and possible combinations of these. Trifluoroacetate salts of peptide compounds described here are typically formed during purification in trifluoroacetic acid buffers using high-performance liquid chromatography (HPLC). Trifluoroacetate salt forms of the peptides described in this invention can be used in various in vitro cell culture studies, assays or tests of activity or efficacy of a peptide compound of interest. The peptide may then be converted from the trifluoroacetate salt by ion exchange methods or synthesized as a salt form that is acceptable for pharmaceutical or dietary supplement compositions.

In another embodiment, the inventive peptides can be prepared using recombinant DNA technology methods wherein an expression vector comprises nucleic acid sequence encoding the peptide of SEQ ID NOS: 1, 2, 3, wherein the nucleic acid sequence is operably linked to a promoter. The expression vector can be delivered to, for example but not limited to, by methods of transformation, transfection, etc, a suitable host cell that allows expression of the peptide. Host cells comprising the expression vector are cultured under appropriate conditions and the peptide is expressed. In one embodiment the host cell is a mammalian cell, including human cell. In another embodiment, the host cell is bacterial, fungal or insect cell. In one embodiment the peptide is recovered from the culture wherein the recovery may include a step that leads to the purification of the peptide. Preparation of the inventive peptides by recombinant technology can be advantageous if the peptides can be post-translationally modified. Further still, a combination of synthesis and recombinant DNA techniques can be employed to produce the amide and ester derivatives of this invention, as well as to produce fragments of the desired polypeptide which are then assembled by methods well known to those skilled in the art.

Expression vectors suitable for nucleic acid sequence delivery and peptide expression in human cells are known in the art. Non-limiting examples are plasmid, viral or bacterial vectors.

Peptides according to the invention may also be prepared commercially by companies providing peptide synthesis as a service (e.g., BACHEM Bioscience, Inc., King of Prussia, Pa.; AnaSpec, Inc., San Jose, Calif.). Automated peptide synthesis machines, such as manufactured by Perkin-Elmer Applied Biosystems, also are available.

The peptides useful in the methods of the present invention are purified once they have been isolated or synthesized by chemical or recombinant techniques. Standard methods for purification purposes can be used, including reversed-phase high-pressure liquid chromatography (HPLC) using an alkylated silica column such as C⁴—, C₂- or C₁₈-silica. In this method, a gradient mobile phase of increasing organic content is used to achieve purification, for example, acetonitrile in an aqueous buffer, usually containing a small amount of trifluoroacetic acid. Alternatively, ion-exchange chromatography can also be used to separate peptide compounds based on their charge. The degree of purity of the peptide compound may be diagnosed by the number of peaks identified by HPLC. A level of peptide purity useful in the invention can result in a single peak on the HPLC chromatogram. In one embodiment, the peptide of interest is at least 94.99% of the input material on the HPLC column. In another embodiment, the peptide of interest is at least 96.99% of the input material on the HPLC column. In one embodiment, the peptide of interest is between 97% and 99.5% of the input material on the HPLC column.

In one embodiment, the peptide of the invention can be delivered in the form of a peptide. In another embodiment, the peptide of the invention can be delivered by an expression vector comprising a nucleic acid encoding the inventive peptide.

Administering the peptide of the invention, in a peptide form or as an expression vector, may be done by a variety of routes or modes. These include, but are not limited to, parenteral, oral, intratracheal, sublingual, pulmonary, topical, rectal, nasal, buccal, sublingual, vaginal, or via an implanted reservoir. Implanted reservoirs may function by mechanical, osmotic, or other means. The term “parenteral”, as used here, includes intravenous, intracranial, intraperitoneal, paravertebral, periarticular, periostal, subcutaneous, intracutancous, intra-arterial, intramuscular, intra-articular, intrasynovial, intrastermal, intrathecal, and intralesional injection or infusion techniques. Such compositions are formulated for parenteral administration, and most for intravenous, intracranial, or intra-arterial administration. When administration is intravenous or intra-arterial, pharmaceutical compositions may be given as a bolus, as separated doses.

A peptide of the invention may be in the form of a sterile injectable preparation, for example, as a sterile injectable aqueous suspension. This suspension may be formulated according to techniques known in the art using suitable dispersing or suspending agents. The sterile injectable preparation may also be a sterile injectable solution or suspension in a non-toxic parenterally acceptable diluent or solvent, for example, as a solution in 1,3-butanediol. Acceptable solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil.

A peptide of this invention may be orally administered via capsules, tablets, caplets, pills, aqueous suspensions, reconstituted lyophilized preparation, and solutions, or syrups. In the case of tablets for oral use, carriers, including lactose and cornstarch, may be used. Lubricating agents, such as magnesium stearate, are also sometimes added. For oral administration in a capsule form, useful diluents include lactose and dried cornstarch. Capsules, tablets, pills, and caplets may be formulated for delayed or sustained release when long-term expression is required.

Alternatively, when orally aqueous suspensions are to be administered, the peptide is advantageously combined with emulsifying and/or suspending agents. Sweetening and/or flavoring and/or coloring agents may be added. In one embodiment the preparation for oral administration provides a peptide of the invention in a mixture that prevents or inhibits hydrolysis of the peptide compound by the digestive system, thereby allowing absorption into the blood stream.

Also, a peptide of this invention may be administered mucosally (e.g. vaginally or rectally). These dosages can be prepared by mixing a peptide of this invention with a suitable non-irritating excipient, which is solid at room temperature but liquid at body temperature and therefore will change states to liquid form in the relevant body space to release the active compound. Examples of these solvents include cocoa butter, beeswax and polyethylene glycols.

Still, for other mucosal sites, such as for nasal or pulmonary delivery, absorption may occur via the mucus membranes of the nose, or inhalation into the lungs. These modes of administration typically require that the composition be provided in the form of a solution, liquid suspension, or powder, which is then mixed with a gas such as air, oxygen or nitrogen, or combinations thereof, so as to generate an aerosol or suspension of droplets or particles. These preparations are carried out according to well-known techniques in the art of pharmaceutical formulation. These preparations may be made as solutions in saline, employing benzyl alcohol or other suitable preservatives, absorption promoters to enhance bioavailability, fluorocarbons, and solubilizing or dispersing agents known in the art.

RNAi Antagonists: The invention further provides nucleic acids which can be used to inhibit the expression of mutant lamin A G608G. Such nucleic acids include but are not limited to siRNA, which inhibit mutant lamin A G608G expression and do not substantially affect the expression of lamin A protein. The sequence of such si RNA molecules comprises the nucleic acid sequence which spans the internal sequence deletion junction which results in the truncated mutant lamin A protein G608G. The G608G protein is called progerin. The sequence and the reading frame of progerin from amino acid 601 to 611 is the following:

nt1801 - GGA GCC CAG AGC CCC CAG AAC TGC AGC ATC ATG - nt 1833
aa601 - S G S G A Q S P Q N C - aa 611

The nucleotide or amino acid positions are given according to the nucleotide or amino acid numbering in the sequence of wild type lamin A. Highlighted in bold are amino acids S656 and Q606 that are now linked in progerin.

To target specific degradation of progerin mRNA, small interfering RNA (siRNA) duplexes within the region described herein can be designed. In one aspect, the invention provides sequences of the type AA(N19)UU, which can be from the open reading frame of the targeted progerin mRNA corresponding to the region from SEQ ID NO:6, wherein N19 denotes any 19 consecutive nucleotides from SEQ ID NO:6 The invention provides the nucleic acids sequence, in 5′ to 3′ direction, of the following N19-mers:

| | |
|---|---|
| GGA GCC CAG AGC CCC CAG A | (SEQ ID NO:7) |
| GA GCC CAG AGC CCC CAG AA | (SEQ ID NO:8) |
| A GCC CAG AGC CCC CAG AAC | (SEQ ID NO:9) |
| GCC CAG AGC CCC CAG AAC T | (SEQ ID NO:10) |
| CC CAG AGC CCC CAG AAC TG | (SEQ ID NO:11) |
| C CAG AGC CCC CAG AAC TGC | (SEQ ID NO:12) |
| CAG AGC CCC CAG AAC TGC A | (SEQ ID NO:13) |
| AG AGC CCC CAG AAC TGC AG | (SEQ ID NO:14) |
| G AGC CCC CAG AAC TGC AGC | (SEQ ID NO:15) |
| AGC CCC CAG AAC TGC AGC A | (SEQ ID NO:16) |
| GC CCC CAG AAC TGC AGC AT | (SEQ ID NO:17) |
| C CCC CAG AAC TGC AGC ATC | (SEQ ID NO:18) |
| CCC CAG AAC TGC AGC ATC A | (SEQ ID NO:19) |
| CC CAG AAC TGC AGC ATC AT | (SEQ ID NO:20) |
| C CAG AAC TGC AGC ATC ATG | (SEQ ID NO:21) |

In another aspect, the invention provides sequences of the type AA(N20)UU, which can be from the open reading frame of the targeted progerin mRNA corresponding to the region from SEQ ID NO:6, wherein N20 denotes any 20 consecutive nucleotides from SEQ ID NO:6. The invention provides the nucleic acids sequence, in 5′ to 3′ direction, of the following N20-mers:

| | |
|---|---|
| GGA GCC CAG AGC CCC CAG AA | (SEQ ID NO:22) |
| GA GCC CAG AGC CCC CAG AAC | (SEQ ID NO:23) |
| A GCC CAG AGC CCC CAG AAC T | (SEQ ID NO:24) |
| GCC CAG AGC CCC CAG AAC TG | (SEQ ID NO:25) |
| CC CAG AGC CCC CAG AAC TGC | (SEQ ID NO:26) |
| C CAG AGC CCC CAG AAC TGC A | (SEQ ID NO:27) |
| CAG AGC CCC CAG AAC TGC AG | (SEQ ID NO:28) |
| AG AGC CCC CAG AAC TGC AGC | (SEQ ID NO:29) |
| G AGC CCC CAG AAC TGC AGC A | (SEQ ID NO:30) |
| AGC CCC CAG AAC TGC AGC AT | (SEQ ID NO:31) |
| GC CCC CAG AAC TGC AGC ATC | (SEQ ID NO:32) |
| C CCC CAG AAC TGC AGC ATC A | (SEQ ID NO:33) |
| CCC CAG AAC TGC AGC ATC AT | (SEQ ID NO:34) |
| CC CAG AAC TGC AGC ATC ATG | (SEQ ID NO:35) |

In another aspect, the invention provides sequences of the type AA(N21)UU, which can be from the open reading frame of the targeted progerin mRNA corresponding to the region from SEQ ID NO:6, wherein N21 denotes any 21 consecutive nucleotides from SEQ ID NO:6. The invention provides the nucleic acids sequence, in 5′ to 3′ direction, of the following N21-mers

| | |
|---|---|
| GGA GCC CAG AGC CCC CAG AAC | (SEQ ID NO:36) |
| GA GCC CAG AGC CCC CAG AAC T | (SEQ ID NO:37) |
| A GCC CAG AGC CCC CAG AAC TG | (SEQ ID NO:38) |
| GCC CAG AGC CCC CAG AAC TGC | (SEQ ID NO:39) |
| CC CAG AGC CCC CAG AAC TGC A | (SEQ ID NO:40) |
| C CAG AGC CCC CAG AAC TGC AG | (SEQ ID NO:41) |
| CAG AGC CCC CAG AAC TGC AGC | (SEQ ID NO:42) |
| AG AGC CCC CAG AAC TGC AGC A | (SEQ ID NO:43) |
| G AGC CCC CAG AAC TGC AGC AT | (SEQ ID NO:44) |
| AGC CCC CAG AAC TGC AGC ATC | (SEQ ID NO:45) |
| GC CCC CAG AAC TGC AGC ATC A | (SEQ ID NO:46) |
| C CCC CAG AAC TGC AGC ATC AT | (SEQ ID NO:47) |
| CCC CAG AAC TGC AGC ATC ATG | (SEQ ID NO:48) |

The different siRNA sequences will be submitted to a BLAST search against the human genome sequence to ensure that no other of the gene of the human genome is targeted. Based on these first criteria 21-nt siRNA will be selected. Other regions of the lamin A cDNA will not be used to prevent inhibition of the expression of the wild type lamin A.

Antisense nucleotide technology has been a described approach in protocols to achieve gene-specific interference. For antisense strategies, stoichiometric amounts of single-stranded nucleic acid complementary to the messenger RNA for the gene of interest can be introduced into the cell. Triple helical nucleic acid structures are also useful for engineered interference. This approach relies on the rare ability of certain nucleic acid populations to adopt a triple-stranded structure. Under physiological conditions, nucleic acids are virtually all single- or double-stranded, and rarely if ever form triple-stranded structures.

In certain embodiments, an RNA interference (RNAi) molecule is used to decrease or inhibit expression of the nucleic acid against which the RNAi is directed. RNAi refers to the use of interfering RNA (iRNA) molecules for example but not limited to double-stranded RNA (dsRNA) or small interfering RNA (siRNA) to suppress the expression of a gene comprising a related nucleotide sequence. RNAi is also referred to as post-transcriptional gene silencing (or PTGS). The sequences inhibitory RNAs are based on the sequence of the target gene, and methods to design iRNAs are known in the art.

RNAi regulates gene expression via a ubiquitous mechanism by degradation of target mRNA in a sequence-specific manner. McManus et al., 2002, Nat Rev Genet 3:737-747. In mammalian cells, interfering RNA (RNAi) can be triggered by 21- to 23-nucleotide duplexes of siRNA. Lee et al., 2002, Nat Biotechnol 20: 500-505; Paul et al., 2002, Nat Biotechnol. 20:505-508; Miyagishi et al., 2002, Nat Biotechnol. 20:497-500; Paddison et al., 2002, Genes Dev. 16: 948-958. The expression of siRNA or short hairpin RNA (shRNA) driven by U6 promoter effectively mediates target mRNA degradation in mammalian cells.

Double-stranded (ds) RNA can be used to interfere with gene expression in many organisms including, but not limited to mammals. dsRNA is used as inhibitory RNA or RNAi of the function of a nucleic acid molecule of the invention to produce a phenotype that is similar to the phenotype of a mutant with decreased expression level and/or activity, for example but not limited to the phenotype of a null mutant of the target.

Many methods have been developed to make siRNA, e.g., chemical synthesis or in vitro transcription. Once made, the siRNA can be introduced directly into a cell to mediate RNA interference (Elbashir et al., 2001, Nature 411: 494-498; Song, E, et al. RNA interference targeting Fas protects mice from fulminant hepatitis. Nat. Med. 2003; 9: 347-351; and Lewis, D L, et al. Efficient delivery of siRNA for inhibition of gene expression in postnatal mice. Nat. Genet. 2002; 32: 107-108). Alternatively, the siRNA can be encapsulated into liposomes to facilitate delivery into a cell (Sorensen, D R, et al. Gene silencing by systemic delivery of synthetic siRNAs in adult mice. J Mol Biol. 2003; 327: 761-766). The siRNAs can also be introduced into cells via transient transfection.

RNAi expression vector”, also referred to herein as a “dsRNA-encoding plasmid”, or shRNA-expressing vector, refers to replicable nucleic acid constructs used to express (transcribe) RNA which produces siRNA moieties in the cell in which the construct is expressed. Such vectors include a transcriptional unit comprising an assembly of (1) genetic element(s) having a regulatory role in gene expression, for example, promoters, operators, or enhancers, operatively linked to (2) a “coding” sequence which is transcribed to produce a double-stranded RNA (two RNA moieties that anneal in the cell to form an siRNA, or a single hairpin RNA which can be processed to an siRNA), and (3) appropriate transcription initiation and termination sequences. The choice of promoter and other regulatory elements can vary according to the intended host cell. Expression vectors of utility in recombinant DNA techniques are often in the form of “plasmids” which refer to circular double stranded DNA loops which, wherein the vector may or may not become integrated in the chromosome. In the present specification, “plasmid” and “vector” are used interchangeably as the plasmid is the most commonly used form of vector. The invention provides for other forms of expression vectors which serve equivalent functions and which become known in the art subsequently.

A number of expression vectors have also been developed to continually express siRNAs in transiently and stably transfected mammalian cells (Brummelkamp et al., 2002, Science 296: 550-553; Sui et al., 2002, PNAS 99(6): 5515-5520; Paul et al., 2002, Nature Biotechnol. 20: 505-508). Some of these vectors have been engineered to express small hairpin RNAs (shRNAs), which get processed in vivo into siRNA-like molecules capable of carrying out gene-specific silencing. In certain embodiments, an shRNA contains plasmid under the control of a promoter, for example a U6 promoter (Paul, C P, et al. Effective expression of small interfering RNA in human cells. Nat. Biotechnol. 2002; 20: 505-508). Another type of siRNA expression vector encodes the sense and antisense siRNA strands under control of separate pol III promoters (Miyagishi and Taira, 2002, Nature Biotechnol. 20: 497-500). The siRNA strands from this vector, like the shRNAs of the other vectors, have 3′ thymidine termination signals. The shRNA gene can be delivered via a suitable vector system, e.g., adenovirus, adeno-associated virus (AAV), or retrovirus (Xia, H, et al. siRNA-mediated gene silencing in vitro and in vivo. Nat. Biotechnol. 2002; 20: 1006-1010; and Barton, G M, et al. Retroviral delivery of small interfering RNA into primary cells. Proc. Natl. Acad. Sci. USA 2002; 99: 14943-14945). In certain embodiments, the invention contemplates use of RNAi vectors which permit stable transfection, and continuous reduction or inhibition of the function of the target protein.

In certain embodiments, the RNA may comprise one or more strands of polymerized ribonucleotide. It may include modifications to the phosphate-sugar backbone or the nucleoside. For example, the phophodiester linkages of natural RNA may be modified to include at least one of a nitrogen or sulfur heteroatom. Modifications in RNA structure may be tailored to allow specific genetic inhibition while avoiding a general panic response in some organisms which can be generated by dsRNA. Likewise, bases may be modified to block the activity of adenosine deaminase. RNA may be produced enzymatically or by partial/total organic synthesis; any modified ribonucleotide can be introduced by in vitro enzymatic or organic synthesis.

The double-stranded structure may be formed by a single self-complementary RNA strand or two complementary RNA strands. RNA duplex formation may be initiated inside or outside the cell. The RNA may be introduced in an amount which allows delivery of at least one copy per cell. Higher doses (e.g., at least 5, 10, 100, 500 or 1000 copies per cell) of double-stranded material may yield more effective inhibition; lower doses may also be useful for specific applications. Inhibition is sequence-specific in that nucleotide sequences corresponding to the duplex region of the RNA are targeted for genetic inhibition. The iRNA molecule may be at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 nucleotides in length.

The RNAi constructs contain a nucleotide sequence that hybridizes under physiologic conditions of the cell to the nucleotide sequence of at least a portion of the mRNA transcript for the gene to be inhibited (i.e., the “target” gene). The double-stranded RNA need only be sufficiently similar to natural RNA that it has the ability to mediate RNAi. Thus, the invention has the advantage of being able to tolerate sequence variations that might be expected due to genetic mutation, strain polymorphism or evolutionary divergence. The number of tolerated nucleotide mismatches between the target sequence and the RNAi construct sequence is no more than 1 in 5 basepairs, or 1 in 10 basepairs, or 1 in 20 basepairs, or 1 in 50 basepairs. Mismatches in the center of the siRNA duplex are most critical and may essentially abolish cleavage of the target RNA. In contrast, nucleotides at the 3′ end of the siRNA strand that is complementary to the target RNA do not significantly contribute to specificity of the target recognition.

Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). In certain embodiments, RNA containing a nucleotide sequences identical to a portion of the target gene are suitable for attenuation and/or inhibition target protein activity. In certain embodiments, RNA sequences with insertions, deletions, and single point mutations relative to the target sequence can be effective for inhibition. Thus, one hundred percent sequence identity between the RNA and the target sequence is not required to attenuate and/or inhibit target activity. Sequence identity between the iRNA and the target of about 99%, 98%, 97%, 96%, 95%, 94%, 93%, 92% or 91% is contemplated of the antagonists and the methods of the present invention. Alternatively, the duplex region of the RNA may be defined functionally as a nucleotide sequence that is capable of hybridizing with a portion of the target gene transcript (e.g., 400 mM NaCl, 40 mM PIPES pH 6.4, 1 mM EDTA, 50° C. or 70° C. hybridization for 12-16 hours; followed by washing).

RNA may be synthesized in vivo or in vitro. Endogenous RNA polymerase of the cell may mediate transcription in vivo, or cloned RNA polymerase can be used for transcription in vivo or in vitro. For transcription from a transgene in vivo or an expression construct, a regulatory region (e.g., promoter, enhancer, silencer, splice donor and acceptor, polyadenylation) may be used to transcribe the RNA strand (or strands). Inhibition may be targeted by specific transcription in an organ, tissue, or cell type; stimulation of an environmental condition (e.g., infection, stress, temperature, chemical inducers); and/or engineering transcription at a developmental stage or age. The RNA strands may or may not be polyadenylated; the RNA strands may or may not be capable of being translated into a polypeptide by a cell's translational apparatus. RNA may be chemically or enzymatically synthesized by manual or automated reactions. The RNA may be synthesized by a cellular RNA polymerase or a bacteriophage RNA polymerase (e.g., T3, T7, SP6). Any suitable method for use and production of an expression construct that are known in the art is contemplated by the present invention as a method to attenuate and/or reduce the target gene activity.

In certain embodiments, the iRNAs are “small interfering RNAs” or “siRNAs.” These nucleic acids can be from about 6 to about 40, 38, 36, 34, 32, 30, 28, 26, 24, 22, 20, 18, 16, 14, 12, 10, 8 nucleotides in length; from about 8 to about 40, 38, 36, 34, 32, 30, 28, 26, 24, 22, 20, 18, 16, 14, 12, 10 nucleotides in length; from about 10 to about 40, 38, 36, 34, 32, 30, 28, 26, 24, 22, 20, 18, 16, 14, 12 nucleotides in length; from about 12 to about 40, 38, 36, 34, 32, 30, 28, 26, 24, 22, 20, 18, 16, 14, nucleotides in length; from about 14 to about 40, 38, 36, 34, 32, 30, 28, 26, 24, 22, 20, 18, 16, nucleotides in length; from about 16 to about 40, 38, 36, 34, 32, 30, 28, 26, 24, 22, 20, 18 nucleotides in length; from about 18 to about 40, 38, 36, 34, 32, 30, 28, 26, 25, 24, 23, 22, 21, 20, 19 nucleotides in length; from about 19 to about 40, 38, 36, 34, 32, 30, 28, 26, 25, 24, 23, 22, 21, 20, nucleotides in length; from about 20 to about 40, 38, 36, 34, 32, 30, 28, 26, 25, 24, 23, 22, 21 nucleotides in length, from about 21 to about 40, 38, 36, 34, 32, 30, 28, 26, 25, 24, 23, 22, nucleotides in length, from about 22 to about 40, 38, 36, 34, 32, 30, 28, 26, 25, 24, 23, nucleotides in length, from about 23 to about 40, 38, 36, 34, 32, 30, 28, 26, 25, 24, nucleotides in length, from about 24 to about 40, 38, 36, 34, 32, 30, 28, 26, 25, nucleotides in length, e.g., corresponding in length to the fragments generated by nuclease “dicing” of longer double-stranded RNAs. The siRNA are double stranded, and may include short overhangs at each end. The overhangs are 1-6 nucleotides in length at the 3′ end. It is known in the art that the siRNAs can be chemically synthesized, or derived from a longer double-stranded RNA or a hairpin RNA. The siRNAs have significant sequence similarity to a target RNA so that the siRNAs can pair to the target RNA and result in sequence-specific degradation of the target RNA through an RNA interference mechanism. The siRNAs can recruit nuclease complexes and guide the complexes to the target mRNA by pairing to the specific sequences. As a result, the target mRNA is degraded by the nucleases in the protein complex. In one embodiment, the 21-23 nucleotides siRNA molecules comprise a 3′ hydroxyl group.

The siRNA molecules of the present invention can be obtained using a number of techniques known to those of skill in the art. For example, the siRNA can be chemically synthesized or recombinantly produced using methods known in the art. For example, short sense and antisense RNA oligomers can be synthesized and annealed to form double-stranded RNA structures with 2-nucleotide overhangs at each end (Caplen, et al. (2001) Proc Natl Acad Sci USA, 98:9742-9747; Elbashir, et al. (2001) EMBO J, 20:6877-88). These double-stranded siRNA structures can then be directly introduced to cells, by passive uptake or a delivery system of choice, such as described herein. In certain embodiments, the siRNA constructs can be generated by processing of longer double-stranded RNAs, for example, in the presence of the enzyme dicer. The siRNA molecules can be purified using a number of techniques known to those of skill in the art. For example, gel electrophoresis can be used to purify siRNAs. Alternatively, non-denaturing methods, such as non-denaturing column chromatography, can be used to purify the siRNA. In addition, chromatography (e.g., size exclusion chromatography), glycerol gradient centrifugation, affinity purification with antibody can be used to purify siRNAs.

In certain embodiments, at least one strand of the siRNA molecules has a 3′ overhang from about 1 to about 6 nucleotides in length, or from about 2 to 4 nucleotides in length, or from about 1 to 3 nucleotides in length. In certain embodiments, one strand having a 3′ overhang and the other strand being blunt-ended or also having an overhang. The length of the overhangs may be the same or different for each strand. In order to further enhance the stability of the siRNA, the 3′ overhangs can be stabilized against degradation. In one embodiment, the RNA is stabilized by including purine nucleotides, such as adenosine or guanosine nucleotides. Alternatively, substitution of pyrimidine nucleotides by modified analogues, e.g., substitution of uridine nucleotide 3′ overhangs by 2′-deoxythymidine is tolerated and does not affect the efficiency of RNAi. The absence of a 2′ hydroxyl significantly enhances the nuclease resistance of the overhang in tissue culture medium and may be beneficial in vivo.

In other embodiments, the RNAi construct is in the form of a long double-stranded RNA. In certain embodiments, the RNAi construct is at least 25, 50, 100, 200, 300 or 400 bases. In certain embodiments, the RNAi construct is 400-800 bases in length. The double-stranded RNAs are digested intracellularly, e.g., to produce siRNA sequences in the cell. However, use of long double-stranded RNAs in vivo is not always practical, presumably because of deleterious effects which may be caused by the sequence-independent dsRNA response. In such embodiments, the use of local delivery systems and/or agents which reduce the deleterious effects are preferred.

In certain embodiments, the RNAi construct is in the form of a hairpin structure (i.e., hairpin RNA). The hairpin RNAs can be synthesized exogenously or can be formed by transcribing from RNA polymerase III promoters in vivo. The hairpin structure can vary in length from about 15 to about 25 nucleotides, from about 15 to about 24 nucleotides, from about 15 to about 23 nucleotides, from about 15 to about 22 nucleotides, from about 15 to about 21 nucleotides, from about 15 to about 20 nucleotides, from about 15 to about 19 nucleotides, from about 15 to about 18 nucleotides, from about 15 to about 17 nucleotides, from about 15 to about 16 nucleotides. Examples of making and using such hairpin RNAs for gene silencing in mammalian cells are described in, for example, Paddison et al., Genes Dev, 2002, 16:948-58; McCaffrey et al., Nature, 2002, 418:38-9; McManus et al., RNA, 2002, 8:842-50; Yu et al., Proc Natl Acad Sci USA, 2002, 99:6047-52). In certain embodiments, such hairpin RNAs are engineered in cells or in an animal to ensure continuous and stable suppression of a gene. It is known in the art that siRNAs can be produced by processing a hairpin RNA in the cell.

In yet other embodiments, a plasmid is used to deliver the double-stranded RNA, e.g., as a transcriptional product. In such embodiments, the plasmid is designed to include a “coding sequence” for each of the sense and antisense strands of the RNAi construct. The coding sequences can be the same sequence, e.g., flanked by inverted promoters, or can be two separate sequences each under transcriptional control of separate promoters. After the coding sequence is transcribed, the complementary RNA transcripts base-pair to form the double-stranded RNA.

The compositions comprising antagonist of the present invention are administered in a therapeutically effective amount, which can be readily determined by one of skill in the art. In certain aspects, therapeutic amount is an amount which leads to inhibition of the target protein level and/or activity. The amount of antagonist which can produce inhibition of the target protein can vary depending on the level of inhibition which is desired. Routine optimization may be involved to ensure that the antagonist can produce the desired level of inhibition. In certain aspects, the level of inhibition can vary from about 95% to about 10% inhibition of the level and/or activity of the target nucleic acid, or protein. In certain aspects, the level of inhibition can vary: from about 95% to about 10%, 15%, 20%, 25%, 30%, 35%, 40%, 45%, 50%, 55%, 60%, 65%, 70%, 75%, 80%, 85%, 90% inhibition of the level and/or activity of the target protein; from about 90% to about 10%, 15%, 20%, 25%, 30%, 35%, 40%, 45%, 50%, 55%, 60%, 65%, 70%, 75%, 80%, 85% inhibition of the level and/or activity of the target protein; from about 85% to about 10%, 15%, 20%, 25%, 30%, 35%, 40%, 45%, 50%, 55%, 60%, 65%, 70%, 75%, 80% inhibition of the level and/or activity of the target protein; from about 80% to about 10%, 15%, 20%, 25%, 30%, 35%, 40%, 45%, 50%, 55%, 60%, 65%, 70%, 75% inhibition of the level and/or activity of the target protein; from about 75% to about 10%, 15%, 20%, 25%, 30%, 35%, 40%, 45%, 50%, 55%, 60%, 65%, 70%, 75%, 70% inhibition of the level and/or activity of the target protein; from about 70% to about 10%, 15%, 20%, 25%, 30%, 35%, 40%, 45%, 50%, 55%, 60%, 65%, inhibition of the level and/or activity of the target protein; from about 65% to about 10%, 15%, 20%, 25%, 30%, 35%, 40%, 45%, 50%, 55%, 60% inhibition of the level and/or activity of the target protein; from about 60% to about 10%, 15%, 20%, 25%, 30%, 35%, 40%, 45%, 50%, 55%, from about 55% to about 10%, 15%, 20%, 25%, 30%, 35%, 40%, 45%, 50%, from about 50% to about 10%, 15%, 20%, 25%, 30%, 35%, 40%, 45%, from about 45% to about 10%, 15%, 20%, 25%, 30%, 35%, 40%, from about 40% to about 10%, 15%, 20%, 25%, 30%, 35%, from about 35% to about 10%, 15%, 20%, 25%, 30%, from about 30% to about 10%, 15%, 20%, 25%, from about 25% to about 10%, 15%, 20%, from about 20% to about 10%, 15%, 15% to about 10%, inhibition of the level and/or activity of the target protein. In certain aspects, the level of inhibition can be about 99%, 98%, 95%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20% inhibition of the level and/or activity of the target protein.

Anti-Progerin Polyclonal and Monoclonal Antibodies

The invention provides an isolated peptide which has the amino acid sequence: GAQSPQNC. A peptide of the sequence GAQSPQNC was synthesized and was used to immunize rabbits for antibody production. The rabbit antibody recognized specifically the mutant lamin A G608G in dermal fibroblast from patients with HGPS. The rabbit antibody did not recognize the wild-type lamin A protein. The rabbit serum containing the antibody works by immunohistochemistry at a dilution 1/500 and by western blotting at a dilution 1/4000.

In one aspect, the invention provides a polyclonal antibody that specifically recognizes progerin, where the polyclonal antibody does not recognize the non-mutated lamin A protein. This tool allowed detection for the first time the truncated lamin A in HGPS fibroblasts carrying the LMNA G608G mutation. Indirect immunofluorescence studies revealed that progerin accumulates gradually within the nuclear compartment reaching an expression level that critically induces a dramatic disruption of the nuclear lamin network. Based on those observations, progerin proteins accumulate within the nucleus in an age-dependent manner (according to the number of population doublings in vitro), and it acts as a dominant negative mutant by altering the nuclear lamin network, which ultimately affects cellular functions such as cell-cycle progression.

In another aspect, the invention is directed to an antibody that specifically binds to a peptide having the amino acid sequence GAQSPQNC. In another aspect, the invention provides an antibody which binds to a polypeptide which comprises the amino acids sequence GAQSPQNC, for example but not limited to a polypeptide such as progerin. In one embodiment, the antibody is polyclonal. In another embodiment, the antibody is monoclonal.

Another aspect of the invention provides antibodies that bind to the peptides of the invention, or a (poly)peptide which comprises the peptide of SEQ ID NO: 1. In another aspect the antibodies of the invention are isolated. In one aspect, the antibodies can neutralize the peptide, and/or the polypeptide which comprises the inventive peptide. In one embodiment, antibodies that specifically bind to the inventive peptide, as a monomer or dimer, may be used in a therapeutic composition to modulate or attenuate the effects of the full length progerin protein. The antibodies of the invention can be monoclonal or polyclonal. Methods for making polyclonal and monoclonal antibodies are well known in the art. Antibodies of the invention can be produced by methods known in the art in any suitable animal host such as but not limited to rabbit, goat, mouse, sheep. The antibodies can be chimeric, i.e. a combination of sequences of more than one species. The antibodies can be fully-human or humanized Abs. Humanized antibodies contain complementarity determining regions that are derived from non-human species immunoglobulin, while the rest of the antibody molecule is derived from human immunoglobulin. Fully-human or humanized antibodies avoid certain problems of antibodies that possess non-human regions which may trigger host immune response leading to rapid antibody clearance. In one embodiment, antibodies can be produced by immunizing a non-human animal with the peptide as a monomer, or a dimer. The immunogenic composition may comprise other components that can increase the antigenicity of the inventive peptide. In one embodiment the non-human animal is a transgenic mouse model, for e.g., the HuMAb-Mouse or the Xenomouse®, which can produce human antibodies. Neutralizing antibodies against the inventive peptide and the cells producing such antibodies can be identified and isolated by methods know in the art.

Making of monoclonal antibodies is well known in the art. In one embodiment, the monoclonal antibodies of the invention are made as follows: spleens were harvested from a rabbit which produced a polyclonal progerin antibody. Harvested cells were fused with the immortalized myeloma cell line partner. After an initial period of growth of the fused cells, single antibody producing clones were isolated by cell purification, grown and analyzed separately using a binding assay (e.g., ELISA, or Western). Two hybridomas were selected based on the ability of their secreted antibody to bind to a peptide of SEQ ID NO:1, including a polypeptide comprising SEQ ID NO:1. Variable regions can be cloned from the hybridomas by PCR and the sequence of the epitope binding region can be determined by sequencing methods known in the art.

The invention provides antibodies and antibody fragments of various isotypes. The recombined immunoglobulin (Ig) genes, particularly the variable region genes, can be isolated from the deposited hybridomas, by methods known in the art, and cloned into an Ig recombination vector that codes for human Ig constant region genes of both heavy and light chains. The antibodies may be generated of any isotype such as IgG1, IgG2, IgG3, IgG4, IgD, IgE, IgM, IgA1, IgA2, or sIgA isotype. The invention provides isotypes found in non-human species as well such as but not limited to IgY in birds and sharks. Vectors encoding the constant regions of various isotypes are known and previously described. (See, for example, Preston et al. Production and characterization of a set of mouse-human chimeric immunoglobulin G (IgG) subclass and IgA monoclonal antibodies with identical variable regions specific for P. aeruginosa serogroup O6 lipopolysaccharide. Infect Immun. 1998 September; 66(9):4137-42; Coloma et al. Novel vectors for the expression of antibody molecules using variable regions generated by polymerase chain reaction. J Immunol Methods. 1992 Jul. 31; 152(1):89-104; Guttieri et al. Cassette vectors for conversion of Fab fragments into full-length human IgG1 monoclonal antibodies by expression in stably transformed insect cells. Hybrid Hybridomics. 2003 June; 22(3): 135-45; McLean et al. Human and murine immunoglobulin expression vector cassettes. Mol Immunol. 2000 October; 37(14):837-45; Walls et al. Vectors for the expression of PCR-amplified immunoglobulin variable domains with human constant regions. Nucleic Acids Res. 1993 Jun. 25; 21(12):2921-9; Norderhaug et al. Versatile vectors for transient and stable expression of recombinant antibody molecules in mammalian cells. J Immunol Methods. 1997 May 12; 204(1):77-87.)

The antibodies of the invention bind to a peptide of SEQ ID NO:1, including SEQ ID NO:1 comprised in a longer polypeptide, for example but not limited to progerin, in a selective manner. As used herein, the terms “selective binding/selectively binds” and “specific binding/specifically binds” are used interchangeably to refer to the ability of the antibody, or antibody fragment to bind to a peptide of SEQ ID NO:1, including a polypeptide comprising SEQ ID NO:1. That is, antibodies that bind selectively to a peptide of SEQ ID NO:1, including a polypeptide comprising SEQ ID NO:1, will not bind to polypeptides which do not comprise the amino acids of SEQ ID NO:1, for example a polypeptide like lamin A, to the same extent and with the same affinity as they bind to a peptide of SEQ ID NO:1, including a polypeptide comprising SEQ ID NO:1. The antibody, or/and antibody fragments, of the invention may bind, if at all, to polypeptides such as lamin A but this binding is with lesser affinity compared to the binding to polypeptides which include the amino acid of SEQ ID NO:1. Lesser affinity may include at least 10% less, 20% less, 30% less, 40% less, 50% less, 60% less, 70% less, 80% less, 90% less, or 95% less.

The present invention provides specific monoclonal antibodies, including but not limited to rabbit, mouse and human, which recognize a peptide of SEQ ID NO:1, including a polypeptide comprising SEQ ID NO:1. When used in vivo in humans, human monoclonal antibodies are far less likely to be immunogenic (as compared to antibodies from another species).

Variable region nucleic acids for the heavy and light chains of the antibodies can be cloned into an human Ig expression vector that contain any suitable constant region, for example (i.e., TCAE6) that contains the IgG1 (gamma 1) constant region coding sequences for the heavy chain and the lambda constant region for the light chains. (See, for example, Preston et al. Production and characterization of a set of mouse-human chimeric immunoglobulin G (IgG) subclass and IgA monoclonal antibodies with identical variable regions specific for P. aeruginosa serogroup 06 lipopolysaccharide. Infect Immun. 1998 September; 66(9):4137-42.) The variable regions can be placed in any vector that encodes constant region coding sequences. For example, human Ig heavy-chain constant-region expression vectors containing genomic clones of the human IgG2, IgG3, IgG4 and IgA heavy-chain constant-region genes and lacking variable-region genes have been described in Coloma, et al. 1992 J. Immunol. Methods 152:89-104.) These expression vectors can then be transfected into cells (e.g., CHO DG44 cells), the cells are grown in vitro, and IgG1 are subsequently harvested from the supernatant. Resultant antibodies will posses human variable regions and human IgG1 and lambda constant regions. In other embodiments, the Fc portions of the antibodies of the invention can be replaced so as to produce IgM.

“Isolated antibodies” as used herein refer to antibodies that are substantially physically separated from other cellular material (e.g., separated from cells which produce the antibodies) or from other material that hinders their use in the diagnostic or therapeutic methods of the invention. The isolated antibodies are present in a homogenous population of antibodies (e.g., a population of monoclonal antibodies). Compositions of isolated antibodies can however be combined with other components such as but not limited to pharmaceutically acceptable carriers, adjuvants, and the like. Methods to isolate and/or purify antibodies are well known in the art.

In other embodiments, the antibody of the invention also includes an antibody fragment. It is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford; and Pier G B, Lyczak J B, Wetzler L M, (eds). Immunology, Infection and Immunity (2004) I.sup.st Ed. American Society for Microbiology Press, Washington D.C.). The pFc′ and Fc regions of the antibody, for example, are effectors of the complement cascade and can mediate binding to Fc receptors on phagocytic cells, but are not involved in antigen binding. An antibody from which the pFc′ region has been enzymatically cleaved, or which has been produced without the pFc′ region, designated an F(ab′)₂ fragment, retains both of the antigen binding sites of an intact antibody. An isolated F(ab′)₂ fragment is referred to as a bivalent monoclonal fragment because of its two antigen binding sites. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd (heavy chain variable region). The Fd fragments are the major determinant of antibody specificity (a single Fd fragment can be associated with up to ten different light chains without altering antibody specificity) and Fd fragments retain epitope-binding ability in isolation. An antibody fragment is a polypeptide which can be targeted to the nucleus. Methods to modify polypeptides for targeting to the nucleus are known in the art and are contemplated by the invention.

The terms Fab, Fc, pFc′, F(ab′)₂ and Fv are employed with their standard immunological meanings [Klein, Immunology (John Wiley, New York, N.Y., 1982); Clark, W. R. (1986) The Experimental Foundations of Modern Immunology (Wiley & Sons, Inc., New York); Roitt, I. (1991) Essential Immunology, 7th Ed., (Blackwell Scientific Publications, Oxford); and Pier G B, Lyczak J B, Wetzler L M, (eds). Immunology, Infection and Immunity (2004) I.sup.st Ed. American Society for Microbiology Press, Washington D.C.].

The CDR regions in humanized antibodies are substantially identical, and can be identical to the corresponding CDR regions of the donor antibody. However, in certain embodiments, it can be desirable to modify one or more CDR regions to modify the antigen binding specificity of the antibody and/or reduce the immunogenicity of the antibody. One or more residues of a CDR can be altered to modify binding to achieve a more favored on-rate of binding, a more favored off-rate of binding, or both, such that an idealized binding constant is achieved. Using this strategy, an antibody having high or ultra high binding affinity of can be achieved. Briefly, the donor CDR sequence is referred to as a base sequence from which one or more residues are then altered. Affinity maturation techniques can be used to alter the CDR region(s) followed by screening of the resultant binding molecules for the desired change in binding. The method can also be used to alter the donor CDR (including, but not limited to a mouse or rabbit CDR), to be less immunogenic such that a potential chimeric (human anti-mouse) antibody response is minimized or avoided. Accordingly, as CDR(s) are altered, changes in binding affinity as well as immunogenicity can be monitored and scored such that an antibody optimized for the best combined binding and low immunogenicity are achieved (see, e.g., U.S. Pat. No. 6,656,467 and U.S. Pat. Pub. Nos: US20020164326A1; US20040110226A1; US20060121042A1).

The antibody of the invention can be used in a variety of applications including but not limited to (a) methods for diagnosing HGPS disease in a subject, (b) methods for screening agents, including but not limited to small molecule drugs, biological agents, in order to identify and monitor agents which can decrease the expression, production, localization, and/or stability of mutant lamin A G608G. The mutant lamin A induces premature senescence in cells. In HGPS patients, vascular cells are the primary cellular targets of the mutant lamin A. Therefore cells derived from an HGPS patient, or from an elderly individual who has been determined to have expression of progerin, can serve as a cellular in vitro model system to test and identify agents that can reduce or inhibit cellular aging, and/or atherosclerosis. Detection of the mutant lamin A using the antibodies of the can be a very important marker to evaluate the potency of such treatments and agents.

The antibody of the invention specifically binds the mutant protein lamin A G608G that is responsible for the premature aging syndrome of young children known as HGPS. Children with HGPS die from wide spread atherosclerosis by the age of 15. The antibody of the invention is useful to understand the mechanisms of atherosclerosis, and can be useful in the design, test and development of new drugs to reduce atherosclerosis. Since, atherosclerosis affects the majority of the elderly population, drugs that can help children can potentially help elders.

In one aspect, the antibody that specifically binds mutant lamin A can be used in a screening method to evaluate drugs designed to affect the levels of expression of mutant lamin A G608G, since the antibody reveals and can be used to quantitate the protein level, including level of expression of mutant lamin A in cells. The effect, including the efficiency and/or potency, of the drug can be addressed by following its effect on the presence, or absence, or change in levels of the mutant A lamin, which can be detected by the antibody of the invention. Furthermore, HGPS is also used as a model system to study the mechanisms of normal aging. Drugs that can reduce or prevent aging can be studied in cells derived from HGPS subject, an elderly subject who has been determined to have expression of mutant lamin A, or genetically modified to express mutant lamin A G608G. In such studies, expression of the mutant lamin A can be a marker to ascertain whether or not a drug can reduce cellular aging.

In one aspect, the invention is directed to methods for diagnosing whether or not a subject has HGPS comprising contacting a sample from the subject with the antibody which recognizes a peptide with amino acid sequence GAQSPQNC and thus the antibody specifically recognizes mutant lamin A G608G. If the antibody binds to the sample, then the subject is deemed to have HGPS. Methods for diagnosing HGPS can include but are not limited to methods for immunohistochemistry, westernblotting or any other molecular approach which can use the antibody of the invention for detecting the mutant lamin A protein.

In another aspect, the invention provides methods for evaluating agents, such as but not limited to drugs, biologicals, and so forth, that affect the level of mutant lamin A G608G protein in a cell or in a subject. Of interest are agents which decrease the level of mutant lamin A protein, and/or agents which change the nuclear localization of progerin. The level of mutant lamin A protein after a treatment with an agent of interest can be monitored by a number of assays, including the assays described herein, which use the antibody of the invention. In another aspect, the invention provides methods for evaluating agents that affect the level of mutant lamin A protein specifically in primary tissues with renewal potential such as muscle, skin and bone.

In another aspect, the invention provides methods for identifying proteins that interact with mutant lamin A G608G and do not interact with wild-type (normal) lamin A protein. In another aspect, the invention provides methods to identify proteins that have lost their ability to interact with mutant lamin A G608G. Such a protein can be identified by comparing the number and identity of proteins that are pulled down in immunoprecipitation experiments that use an antibody which binds specifically to mutant lamin A G608G or an antibody which binds to lamin A protein. Absence of specific proteins in the pull downs with the antibody which binds specifically to mutant lamin A G608G as compared to the antibody against normal lamin A will provide proteins that no longer interact with mutant lamin A G608G. Presence of specific proteins in the pull downs with the antibody which binds specifically to mutant lamin A G608G will provide proteins that interact with mutant lamin A G608G and do not interact with lamin A protein.

### EXAMPLES

Characterization of an anti-lamin A G608G antibody: The lamin A G608G amino acid sequence reading frame was determined previously (De Sandre-Giovannoli et al, 2003; Paradisi et al, 2005). To generate a specific anti-Lamin A G608G antibody, a short peptide (8 residues, GAQSPQNC) was chosen overlapping the region where the 50 residues truncation occurred in the lamin A mutant G608G (FIG. 9). The peptide was purchased from BioSynthesis Inc.; Cocalico Biological Inc. and two rabbits were immunized. Preimmune and immune sera were characterized by Western blot and by indirect immunofluorescence on HGPS and control fibroblasts.

Primary dermal fibroblast cells: Three primary cultures of dermal fibroblasts derived from HGPS patients carrying the LMNA mutation G608G: HGADFN001, HGADFN003, and HGAFN127 from the Progeria Research Foundation; a fourth culture is described herein PT001. In parallel, three previously established control dermal fibroblast cultures were used. Exemplary analyses shown in the figures correspond to HGPS fibroblasts HGADFN127.

Indirect immunofluorescence and Western blot: Primary cultures of dermal fibroblasts were processed for indirect immunofluorescence as described previously (Paradisi et al, 2005). Rabbit antibodies directed against A-type and B-type lamins were described previously (Chaudhary et al, 1993; Cance et al, 1992). Goat anti-lamin B1 M-20 and anti-prelamin A C-20 (Santa Cruz), monoclonal antibodies anti-emerin 4G5 (Novocastra), anti-nucleoporin Nup153 clone 414 (Covance), anti-lamin A Jo14 (Serotec), and anti-Ki-67 (BD Bioscience) were purchased. Secondary antibodies were affinity purified Alexa Fluor 488 goat or donkey IgG antibodies (Molecular Probes) and Cy3-conjugated IgG antibodies (Jackson ImmunoResearch laboratories). All samples were counterstained with DAPI (Sigma-Aldrich). When cells were treated daily for 3 days with farnesyltransferase inhibitor FTI-277 (Calbiochem), the culture medium was supplemented with 20 μM FTI-277 or DMSO prior to immunofluorescence analysis as described previously (Adjei et al, 2000). Images were processed using Adobe Photoshop. For Western blot analysis, total cellular protein extracts were isolated from dermal fibroblast cultures at different population doublings (25 to 55) and processed as described previously (Paradisi et al, 2005).

In vivo detection of lamin A G608G on HGPS skin biopsy sections: Immunohistochemistry was performed on frozen skin sections from a biopsy of a 9 year-old donor with HGPS carrying the LMNA G608G mutation (HGADFN143) provided by the Progeria Research Foundation, and normal skin sections from an 86 year-old unaffected individual. 6 μm frozen sections were fixed by methanol/acetone (IV/IV) at −20° C. for 10 minutes and processed for indirect immunofluorescence as described herein. Rabbit polyclonal anti-lamin A G608G serum and corresponding preimmune serum (PIM) were used at dilution 1/400, and anti-lamin A/C antibody at 1/600(Chaudhary and Courvalin, 1993). Anti-human a smooth muscle actin, clone 1A4 (DakoCytomation) and anti-CD-31/PECAM-1 antibodies (Lab Vision) were used according to the manufacturers. One section was stained with hematoxylin and eosin for morphological comparison.

Cell migration assay: Cell migration assays were performed using standard 8.0 μm pore size transwell inserts (BD Bioscience). Subconfluent HGPS and control dermal fibroblasts from population doublings (35 to 40) were harvested and resuspended in serum-free DMEM (5×10⁵ cells/ml). 500 μl of cell suspension was added to the upper chamber of the inserts. The lower chamber was filled with 750 μl of DMEM containing 15% FCS. After 22 hours incubation at 37° C., the filters were removed, and the membranes were processed for upper or lower membrane surfaces for indirect immunofluorescence analysis with anti-lamin A G608G and anti-emerin antibodies.

Senescence-associated β-Galactosidase staining: Dermal fibroblast cultures from population doublings (PPDs) 40-45 were stained for senescence β-Galactosidase (SA-β-Gal) activity at pH6 (Dimiri et al, 1995). Cells grown on coverslips were fixed in methanol/acetone at −20° C. for 5 min, washed in PBS and incubated with anti-lamin A G608G antibody for one hour, washed in PBS, and then incubated overnight in fresh SA-β-Gal staining solution (1 mg of 5-btomo-4-chloro3-indolyl β-D-galactoside (X-Gal) per milliliter of buffer containing: 40 mM citric acid sodium phosphate, pH6/0.5 mM potassium ferrocyanide/5 mM potassium ferricyanide/150 mM sodium chloride/2 mM magnesium chloride (Dimiri et al, 1995). After a 15-hour incubation at room temperature, cells were washed in PBS and further processed for indirect immunofluorescence with the secondary antibody.

Quantification: The percentages of ki-67 positive cells, of misshapen or blebbing nuclei, and senescence β-gal positive cells were determined by direct count of 300 cells per coverslip in triplicate, and from three independent experiments. The data sets were plotted using Kaleidagraph.

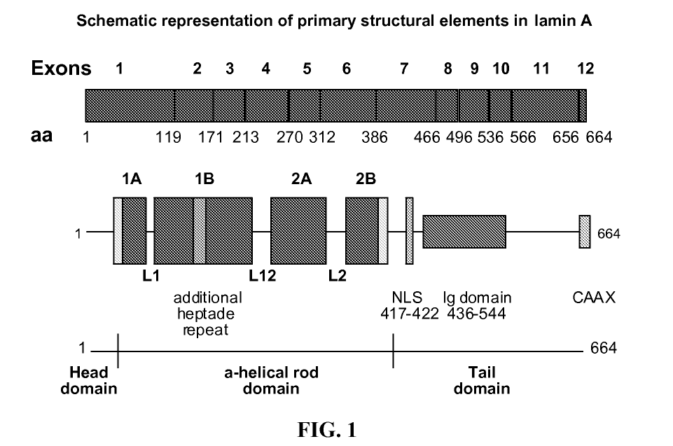

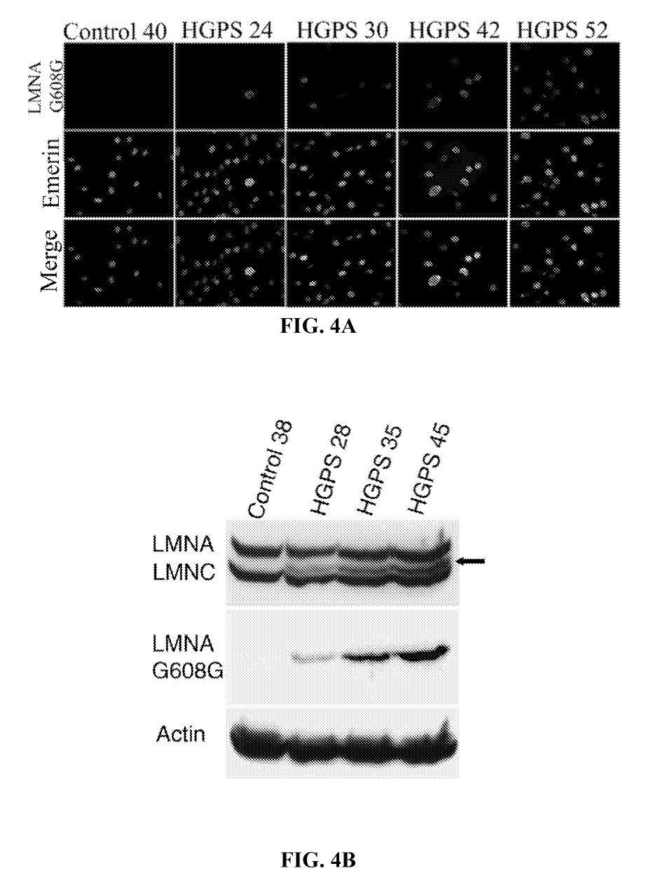

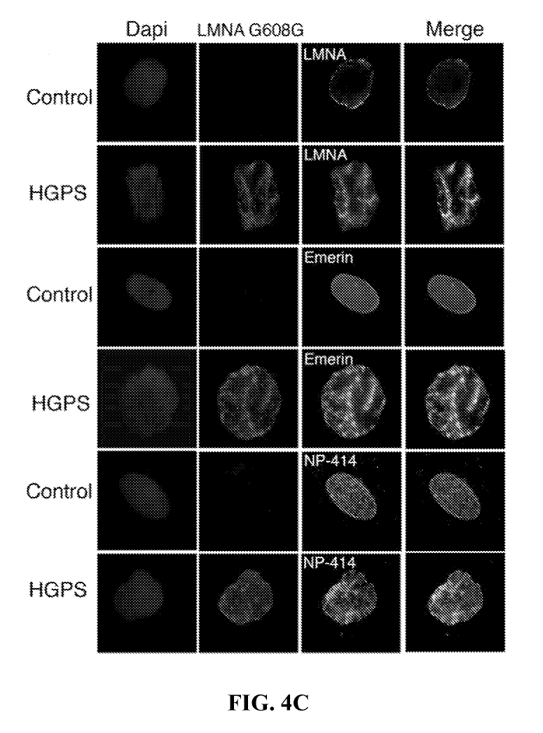

Mutant Lamin A G608G accumulates within the nucleus in a cellular age dependent manner as detected by a specific antibody: A rabbit polyclonal antibody was raised against a short peptide overlapping the 50 amino acid deletion region in the mutant lamin A G608G (progerin, FIG. 9) (Paradisi et al, 2005). The immune sera, denoted anti-LMNA G608G, were tested on primary dermal fibroblast cultures at early and late population doublings (PPDs, 20-50), using four different HGPS fibroblast cultures. All four behaved similarly in vitro; therefore exemplary analyses described herein are based on HGPS human fibroblasts HGAFN127. Only few cells were positively labeled with the anti-LMNA G608G antibody at early PPDs (below 25, FIG. 1A), while nuclei were labeled with anti-emerin antibody that detects an inner-nuclear membrane protein (FIG. 4A) (Bione et al, 1994). The number of nuclei positively stained with anti-LMNA G608G increased with the number of cellular generations in vitro. Signal intensity varied: in some cells it was faint, in others very bright, indicating that the HGPS fibroblasts contained different amounts of progerin (FIG. 4A). Primary dermal fibroblast cultures are asynchronous, indicating that heterogeneity of anti-LMNA G608G staining reflected cellular age (Van Gansem and Van Lerberghe, 1988). Indeed, Western blot analysis indicated that the progerin product was increased in cultures at late PPDs (FIG. 4B), as previously reported using anti-A-type lamin antibodies (Goldman et al, 2004). Anti-LMNA G608G serum gave no signal in control dermal fibroblasts by indirect immunofluorescence or by Western blotting (FIGS. 4A, B).

Progerin accumulation induces nuclear envelope invaginations: Careful analysis of brightly labeled nuclei with anti-LMNA G608G revealed that the truncated lamin A was distributed in dot-like or cable-like structures (FIG. 4. The intra- and transnuclear cable-like structures detected with anti-LMNA G608G were also labeled with emerin or nucleoporin 414 antibodies, indicating that they associated with the membrane compartment. Furthermore, DAPI staining of mutant nuclei showed severe chromatin rearrangement with the formation of intrachromatin folds that coincided with anti-LMNA G608G staining (FIG. 4C. These results indicated increased nuclear envelope (NE) invaginations. In nuclei containing low levels of progerin, the mutant protein colocalized with wild-type lamin A and lamin B1 within the nuclear lamina, and emerin and nucleoporin 414 antibodies showed a normal distribution pattern.

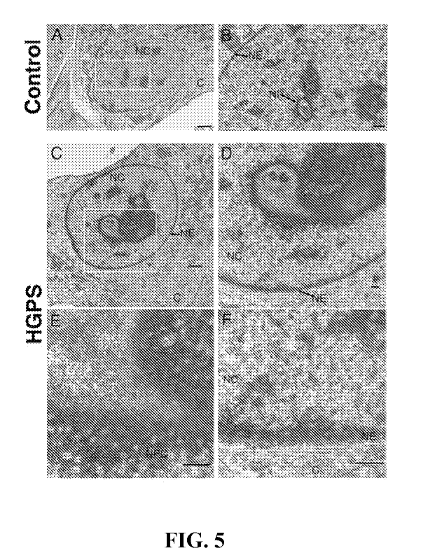

To determine that the cable-like structures were indeed NE invaginations, electron microscopy of HGPS cultures was performed at PPDs 38, where a significant number of cells (25 to 30%) harbored progerin cable-like distribution as detected by indirect immunofluorescence. Electronmicroscopy of ultrathin HGPS sections revealed that 57% of the nuclei showed drastic NE deformations altering the nucleus from a spherical to a lobulated shape; some nuclei remained roughly ovoid but harbored significant intranuclear invaginations (FIG. 5C). Those numerous invaginations were composed of a double membrane containing nuclear pores, with some regions exhibiting high pore density; they also remained in close apposition with the nuclear lamina and the chromatin (FIG. 5D). In contrast, ultrathin sections of control fibroblast cultures at similar or higher PPDs had approximately 22% of the nuclei exhibiting NE invaginations, none as deep or as numerous as in HGPS cells (FIG. 5A). Increased NE invaginations have been reported in cells permanently overexpressing prenylated lamins or components of the NE (lamin B receptor or nucleoporin 153), which resembled the ones observed in HGPS fibroblasts at late PPDs (Prufert et al, 2004; Ralle et al, 2004).

Farnesyltransferase inhibitor normalized nuclear morphology in HGPS dermal fibroblasts: Normal prelamin A processing involves an orchestrated sequence of modifications: farnesylation, carboxyl-methylation of the cysteine residue within the CSIM motif, proteolytic cleavage of SIM, and finally cleavage of 15 residues along with its farnesyl moiety to generate the mature lamin A (Weber et al, 1989). Since the last cleavage cannot occur in LMNA G608G, progerin retains its farnesyl group, stabilizing progerin interactions with the inner nuclear membrane (Mallampalli et al, 2005; Capell et al, 2005; Glynn et al, 2005; Yang et al, 2005). Thus, progerin can force the inward growth of the NE to increase the surface area that can accommodate the excess progerin accumulation in HGPS.

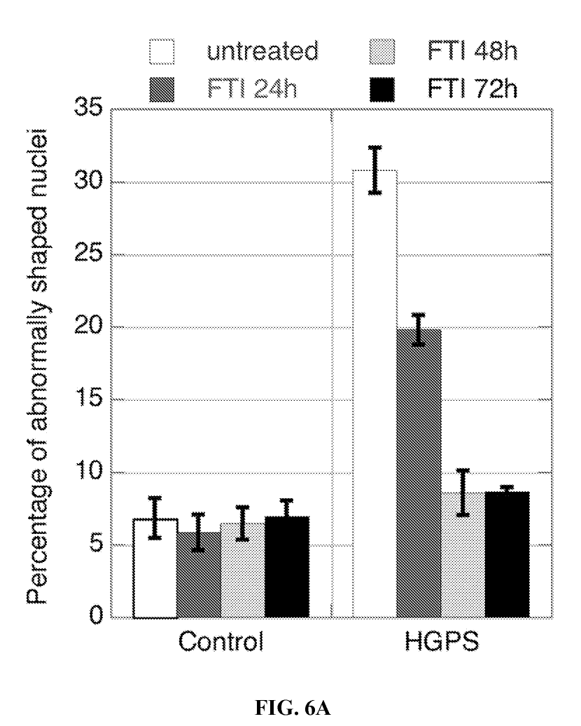

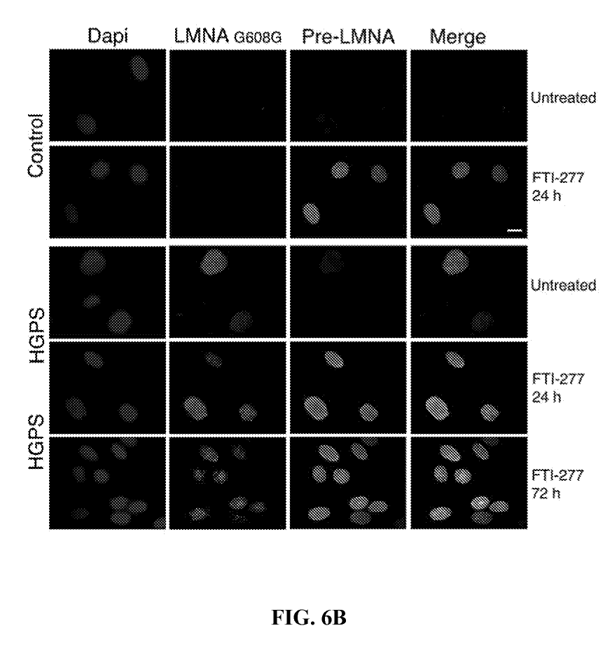

To determine the role of farnesylation in HGPS nuclear abnormalities, control and HGPS fibroblast cultures (PPDs 35-38) were treated daily with 20 μM farnesyltransferase inhibitor FTI-277 (Adjei et al, 2000). After 48 h treatment, the percentage of HGPS nuclei with abnormal shapes decreased from 31% to 8% (FIG. 6A). The intranuclear repartition of prelamin A and the localization of LMNA G608G differed greatly between treated and un-treated cells; prelamin A was not detectable in control or HGPS cells (FIG. 6B). When farnesylation was inhibited in control cells, the lamin A precursor was present in nuclear foci but mostly accumulated in the lamina where it is assembled, giving rise to a strong nuclear rim staining (FIG. 6B) as previously reported (Adjei et al, 2000; Sinensky et al, 1990; Sasseville et al, 1995). In HGPS fibroblasts treated with FTI-277, the nuclei appeared overall more ovoid and wild-type prelamin A localization resembled that in control cells (FIG. 3B). After 24 h treatment, anti-LMNA G608G stained the nuclear rim in HGPS fibroblasts, indicating that progerin was still incorporated into the nuclear lamina. Progerin was also detected in nuclear foci similarly to wild-type prelamin A (FIG. 6B). However, after 48 h FTI-277 treatment, progerin was progressively depleted from the NE and lamina. Progerin formed large aggregates within the nucleoplasm that sometimes overlapped with the wild-type prelamin A. 76 h after treatment, while wild-type prelamin A was still predominantly detected at the NE and in some nucleoplasmic foci (FIG. 6B), progerin was totally depleted from the NE in most nuclei and was localized within nucleoplasmic aggregates (FIG. 6B). These results show that unfarnesylated progerin lost its ability to bind the nuclear envelope and to assemble into the lamina, thus restoring a more regular nuclear shape for the majority of the HGPS cells (FIG. 6).

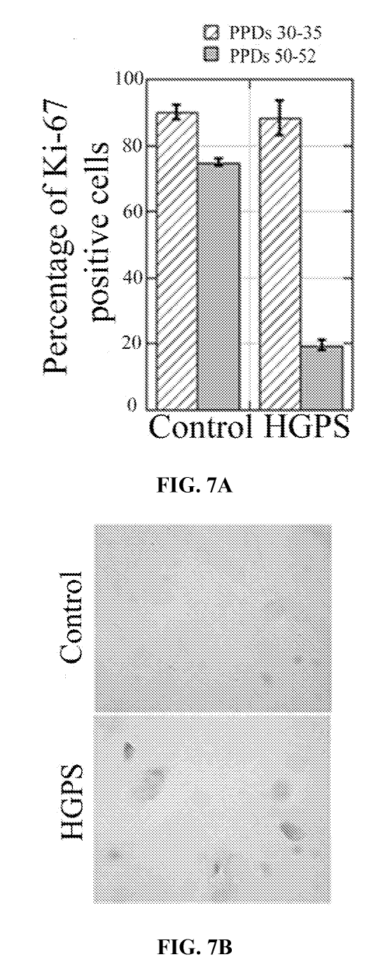

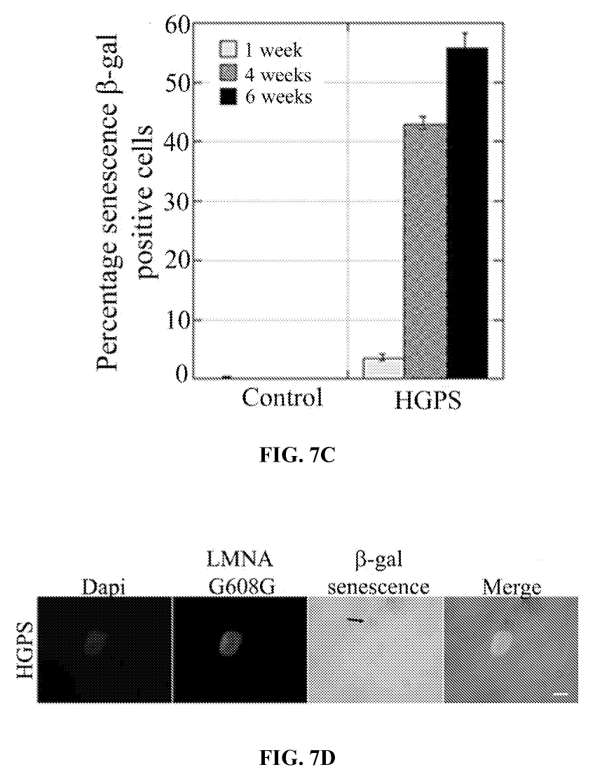

Mutant lamin A induces decreased cellular proliferation premature senescence and altered motility: During the spatio-temporal expression studies of mutant LMNA G608G, it was noticed that HGPS fibroblasts HGAFN127, grew at the same rate as control cells until they reached approximately 40-45 PPDs (FIG. 7A). Between 40-50 PPDs, as HGPS cells accumulated progerin, their growth rate decreased rapidly until they ceased dividing between PPDs 52-57 under the culture conditions used herein. Late passage control fibroblasts continued to grow and exhibited only a slow decline in their proliferative index when compared to early PPDs (FIG. 7A). These observations prompted examination whether HGPS cells were entering premature senescence.

Primary cultured cells undergoing cellular senescence in vitro express increased beta-galactosidase (β-gal) activity when assayed at pH 6 (Dimiri et al, 1995). Indeed, when senescence β-gal positive cells were scored at late PPDs in HGPS cells after one week in culture, 3% of the cells were positive while none were detected in control cultures at the same PPDs (FIG. 7C). Furthermore, the number of senescent HGPS cells increased with time in vitro, reaching 43% after 4 weeks (FIG. 7C). In control cells after 4 weeks, 1 to 2 senescence β-gal positive cells can be detected on entire coverslips. These results indicate that senescent cells accumulated prematurely in HGPS fibroblast cultures. Moreover, when senescence-associated β-gal activity and LMNA G608G levels were assayed on late PPDs of HGPS cells, senescent cells (β-gal positive) were brightly labeled with anti-LMNA G608G antibody and harbored nuclear cable-like structures reminiscent of NE invaginations (FIG. 7D). This indicates that HGPS fibroblasts, concomitantly with the accumulation of mutant lamin A, withdraw from the cell cycle via the apoptotic pathway as previously reported (Bidger and Kill, 2004) or senescence (FIG. 7D).

To determine the cellular effects of progerin, cellular migration, which is an important function in a variety of physiological aspects was examined. Examination was done in a migration assay where fibroblasts can demonstrate both inherent and directional motility in response to a chemotatic stimulus (Li et al, 1997). While the overall number of migrant HGPS cells was similar to controls, none of the migrant HGPS cells were strongly labeled with anti-lamin A G608G antibody (FIG. 7E). Indeed, LMNA G608G brightly positive cells remained on the upper face of the filters where they had been seeded (FIG. 7E), associating high progerin levels with reduced migration.

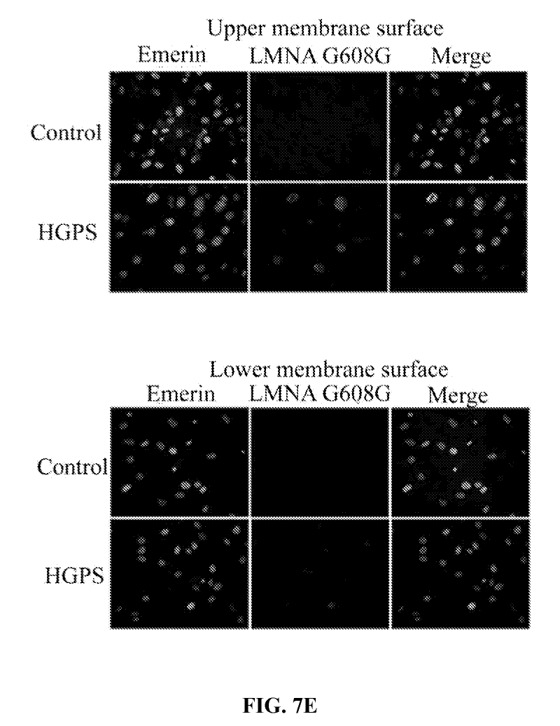

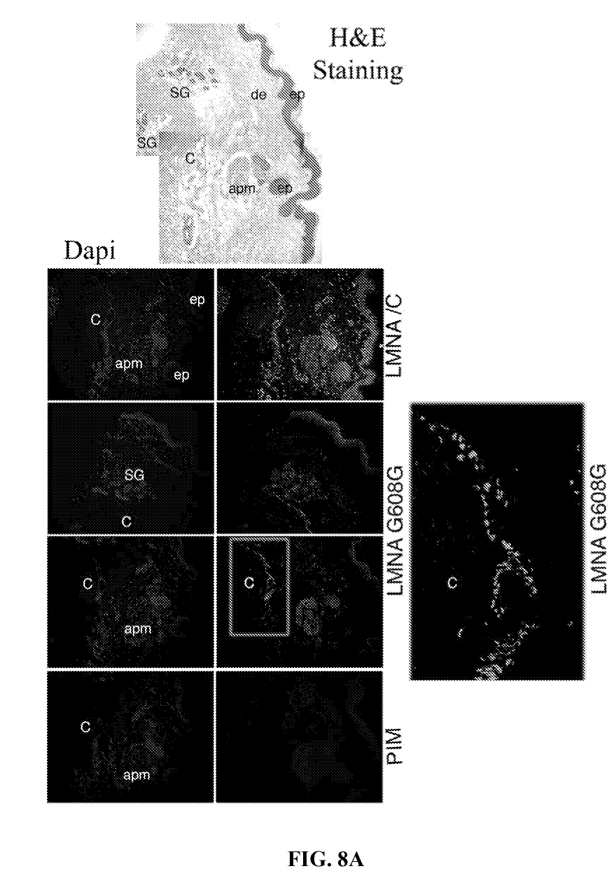

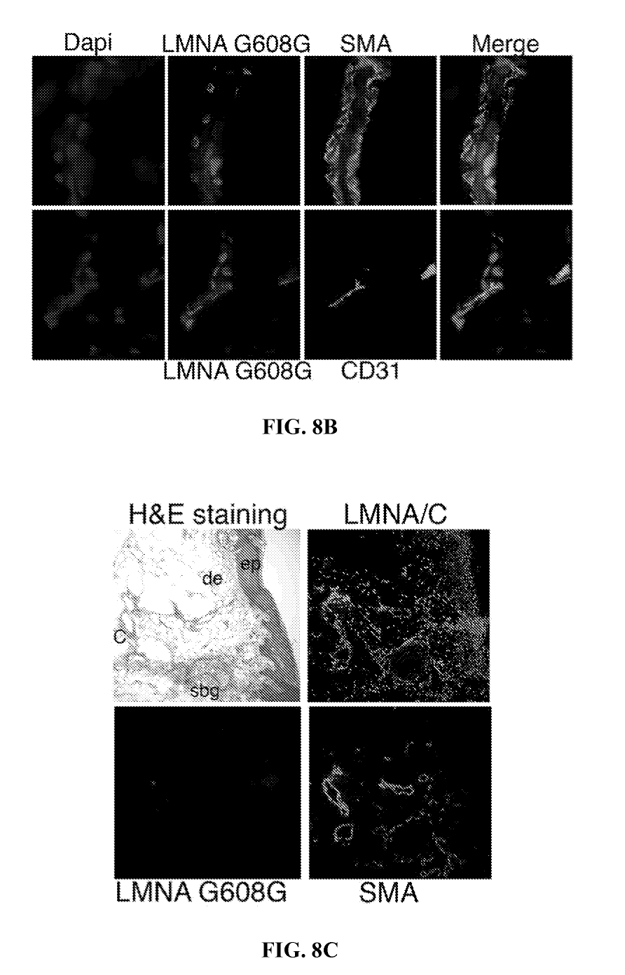

Vascular cells are the primary targets for progerin build up, linking the mutation to the pathogenesis of atherosclerosis in HGPS. Understanding the cellular mechanism underlying the clinical sequelae of HGPS requires the identification of the cellular targets of progerin in vivo. Immunohistochemistry was performed on serial frozen sections from a skin biopsy derived from a 9 year-old subject with HGPS (HGADFN143). Hematoxylin and eosin staining showed a well-organized epidermis, a large blood vessel, sweat glands, an arrector pili muscle, and absence of hair follicle (FIG. 8A). Connective tissue was very dense with thick fibrous structures throughout the dermis. Subcutaneous fat tissue was absent in this sample. Lamin A/C was detected in the nuclei of most cells from the epidermis and dermis (FIG. 8A). The anti-LMNA G608G antibody showed a very restricted pattern of distribution, with positively labeled nuclei localized within the blood vessels, some cells surrounding the sweat glands and in arrector pili muscle. In the epidermis, very few keratinocytes were positively stained with anti-LMNA G608G in the uppermost layer of the epidermis. Immunodetection with the LMNA G608G preimmune serum gave no signal, indicating that the signal obtained with anti-LMNA G608G antibody was specific. The anti-LMNA G608G antibody also gave no signal on skin sections from an 86 year-old unaffected individual (FIG. 8C).

To identify the cell type(s) positively labeled in the blood vessels, HGPS skin sections were double labeled with anti-LMNA G608G antibody and with anti-α smooth muscle actin, a reliable marker of smooth muscle cells, or anti-CD31 directed against the endothelial cell surface antigen CD31. The brightest signal obtained with anti-LMNA G608G was indeed located within the vascular system, and that the positively labeled nuclei were primarily smooth muscle and endothelial cells (FIG. 8B). At high magnification, smooth muscle cell nuclei with a strong LMNA G608G signal adopted similar cable-like structures as HGPS fibroblasts reminiscent of the NE invaginations. This is direct in vivo evidence for the presence of progerin in vascular cells. This is also evidence for a direct relation between progerin and atherosclerosis in HGPS.

Autopsy of a few HGPS cases has demonstrated severe smooth muscle cell depletion in atherosclerotic aorta media (Stehbens et al, 1999; Stehbens et al, 2001). The rare remaining smooth muscle cells had aberrant cellular shape, ballooning mitochondria, and increased cytoplasmic density (Stehbens et al, 1999). Those observations indicated that smooth muscle cells in HGPS were hypersensitive to hemodynamic and ischemic stress, and can become defective in restoring vascular integrity after injury (Stehbens et al, 1999). In uninjured blood vessels, smooth muscle and endothelial cells are mostly quiescent. In HGPS subjects, vascular smooth muscle cells can be subjected to increased mechanical stress forcing them to undergo several cellular divisions to regenerate vascular tissue. The build up of nuclear progerin can occur as cells reach a high number of PPDs. This can be the cause of the detected progerin accumulation in vascular cell nuclei of HGPS skin sections. In such a setting, cells will progressively lose their capacity to grow and migrate, and will become apoptotic or senescent. Progerin-dependent cellular changes might contribute to the vascular deterioration responsible for the progression of atherosclerosis in HGPS subjects.

The analyses described herein show in vivo cellular and tissue localization of a mutant lamin A (progerin) responsible for severe, premature atherosclerosis in HGPS. Progerin accumulates primarily in vascular cells and can be regarded as a key player in the onset of atherosclerosis, the primary cause of death for HGPS patients. In certain aspects, the invention provides that methods for restoring or improving normal cellular function by preventing progerin accumulation, expression or posttranslational modification using treatments such as FTI, agents identified by the methods of the current invention, genetic therapies including inhibition/attenuation of progerin express or activity (Scaffidi et al, 2005) can be used in treatments that can reduce disease progression in HGPS subjects, including children. In another aspect, the invention provides that methods for restoring or improving normal cellular function as contemplated herein can be used in treatments that can reduce and/or prevent ageing in individuals who are not affected by HGPS.

Primary dermal fibroblast cultures and cellular passage number conditions: Cells are grown in DMEM medium supplemented with 15% fetal calf serum, 2 mM glutamine, 10 U/ml penicillin and 50 μg/ml streptomycin, at 37° C. with 5% CO₂. Cells are grown in 10 cm dishes and are collected when the fibroblast cells cover 70% of the dish. Dishes are treated with EDTA/trypsin solution, cells are collected, and the number of cells is determined. The cell pellet is resuspended in complete medium and split into 4 dishes. The cellular passage number is defined as the number of times that cells reaching 70% confluency are submitted to EDTA/trypsin and split into 4 dishes. After each trypsin treatment the passage number is increased to N+1. By determining the number of cells seeded on the dish and collected when they reach 70% confluency, the number of population doublings was as 2, using the standard formula (population doublings=Log 10NH−Log 10NS/Log 102, where NH is the number of cells harvested and NS is the number of cells seeded) (Wallis et al., 2004). Under these culture conditions, the population doublings will correspond to the cellular passage number multiplied by 2.

Nuclear envelope disorganization in dermal fibroblasts PT001: The morphology of primary cultures of skin fibroblasts from control individuals and from the subject PT001 were examined at cellular passages below 10. Confocal microscopy revealed several abnormalities in the nuclei from the patient, mainly in the shape and the size of the nucleus, in approximately 19% of the cells. Since no defect was detected in the A- or B-type lamin network, as both nuclear lamins followed the nuclear envelope contour, A- and B-type lamins repartition as well as A-type and emerin localization were examined by double immunofluorescence approach. The most abnormal nuclei from the proband were screened and analyzed for the localization of the lamin B1 and emerin in comparison to the A-type lamin. FIG. 10 shows the lamina localization of the three components and reflects the overall localization of the different proteins observed in the patient's most dysmorphic nuclei. The immuno-staining of the A- and B-type lamins are superimpo-sable (FIG. 10). It was also noted that even at sites of nuclear envelope blebbing or herniation, both A- and B-type lamins had similar repartitions. Emerin, too, was detected at the same sites as the A-type lamin and followed exactly the same localization pattern. When intra-nuclear lamin A spot-like staining was detected, emerin was in the same intranuclear structures (FIG. 10). The DAPI staining did not indicate the presence of a detachment between the nuclear lamina and the chromatin even in the most abnormally shaped nuclei as observed by light microscopy (FIG. 10, panels DAPI). The lamin A, B1 and emerin staining was in close apposition to the DNA labeling signal, indicating that the envelope and nuclear lamina remained in close contact with the chromatin (FIG. 10). These results indicate that, even in the most altered nuclei from the HGPS subject, the mutant lamin A does not interfere dramatically with the nuclear lamina network. During the time course of this study, there was no noticeable difference in the growth rate of the patient fibroblasts versus the control cells at early passage number (below passage 10).

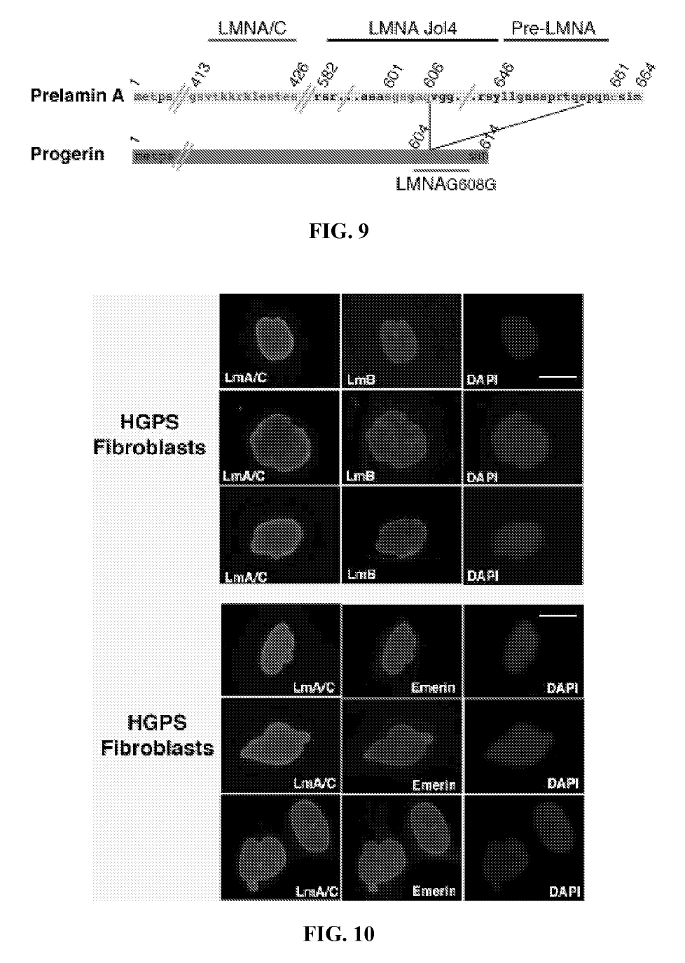

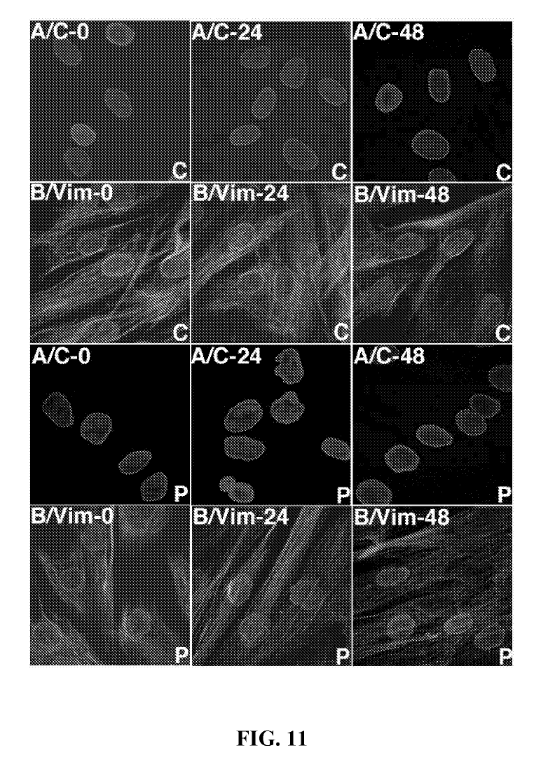

Dermal fibroblasts from the HGPS patient are hypersensitive to heat stress: To evaluate the resistance to stress of the nuclear envelope, fibroblasts from the HGPS patient and control cells were submitted to heat-shock treatment for 30 minutes at 45° C., then either fixed immediately (time 0) or incubated at 37° C. and left to recover from the stress for 24 and 48 hours. The nuclear shape was examined after labeling with anti-lamin A/C or anti-lamin B1 antibodies at the different time points of recovery (0, 24, and 48 hours; FIG. 11). In control cells, the nuclear shape and the A and B-type lamin distribution in control cells were not modified by heat-shock treatment (FIG. 11, panels C), whereas nuclear deformation appeared in the HGPS patient cells (FIG. 11, panels P). Patient cells fixed immediately after heat shock showed an increase number of cells with altered nuclei; the nuclear envelope was deformed, and many nuclei had a ruffled appearance (FIG. 11, panel P, 0). Pleats or folds in the nuclear envelope accompanied the nuclear deformations, and the number of irregular nuclei increased to nearly 70% in the patient cells after 24 hours, determined by direct count of a total of 1000 nuclei observed on different coverslips at time point 24 hours recovery (FIG. 11, panels P 24). Moreover, some nuclei were more severely affected and showed some budding or nuclear lobe formation after 24 hours (FIG. 11, panel P A/C-24). This type of nuclear damage was not observed in the control cells (FIG. 11, panels C). After 48 hours recovery from the stress, patient nuclei with invaginations were no longer found, indicating that these cells died and had been detached during the immuno-fluorescence procedure. The patient fibroblasts appeared to have recovered from stress after 48 hours, since the nuclei were less ruffled, and the irregularities resembled those observed prior to the heat shock (FIG. 11, panels P, 48). The cytoplasmic compartment from HGPS and control cells did not change dramatically since no rearrangement of the cytoplasmic intermediate filament network was noticed after heat shock treatment or at the different periods of recovery (FIG. 11, panels Vim).

The survival rate of control and patient heat-shocked fibroblasts was evaluated and compared with unheated cells (Table 2). The control cells recovered rapidly since no changes were apparent in their growth rate after 24 and 48 hours. However, the fibroblasts from the HGPS patient PT001 were very sensitive to the heat shock treatment; the number of dysmorphic nuclei was very high and showed dramatic abnormalities (FIG. 11, panels HGPS). In addition, their recovery from stress was only observed after 48 hours when the cells started to increase in number, indicating they had re-entered mitosis. These findings indicate that the patient fibroblasts have a delayed recovery in response to heat shock treatment. Another HGPS primary culture, HGADFN127, from the Progeria Research Foundation, was examined and it demonstrated a similar response to the heat shock treatment to that of fibroblast culture PT001. Altogether these experiments indicate that HGPS fibroblasts are hypersensitive to heat stress and that their response to stress was delayed by 24 hours compared to the control cells, demonstrating defective cellular stability. Further analysis can determine whether there are changes in the expression levels of heat shock proteins under normal and stress conditions to define the causes of this defect.

TABLE 2
Average percentages of fibroblasts collected from 10 cm
tissue culture dishes prior or after heat shock treatment for
a period of 30 minutes at 45° C. and allowed to recover at
37° C. for 0, 24 and 48 hours. Averages were estimated from
three independent experiments using HGPS PT001 cells.
% of fibroblasts recovered after heat treatment

| | Before | Hours after heat shock | | |
|---|---|---|---|---|
| | heat shock | 0 | 24 | 48 |
| Control | 100 | 97.6 ± 0.9 | 139.2 ± 1.5 | 175.1 ± 2.9 |
| HGPS | 100 | 95.8 ± 1.5 | 74.6 ± 5.1 | 104.9 ± 5.8 |

Generation of the full-length progerin cDNA by RT-PCR: Total RNA from PT001 fibroblast culture was prepared using Trizol solution (Invitrogen). Progerin cDNA was generated using one-step RT-PCR reaction with superscript kit from Invitrogen and with specific primers containing restriction site EcoR1 and Kpn1 to allow direct subcloning into PEGFP-C1 vector. The sequence of the forward primer was as follows: 5′CCGGAATTCTATGGAGACCCCGTCC 3′ (SEQ ID NO: 4). The sequence of the reverse primer was as: 5′CGGGGTACCCATGATGCTGCAG 3′ (SEQ ID NO: 5). A flag tagged progerin construct using PSVK3 vector was also generated. Constructs were verified by direct sequencing. The flag tagged progerin construct can be used to generate new constructs such as His tagged progerin construct.

Production and characterization of anti-progerin antibody: The progerin amino acid sequence reading frame as shown in FIG. 3 reads as follows from amino acid 601 to the last residue: SGSGAQSPQNCSIM and is in accordance with the published sequence (De Sandre-Giovannoli et al., 2003). To generate a specific anti-progerin antibody, a short peptide (8 residues) was chosen overlapping the region where the 50 amino acids truncation occurred in progerin sequence. The peptide was synthesized by BioSynthesis Inc. The coupling of the peptide and the immunization of two rabbits following the CBI standard protocol were performed by Cocalico Biological, Inc. Prior to immunization, 5 pre-immune serums corresponding to rabbits K1 to K5 were screened, by indirect immunofluorescence on HGPS fibroblast cells. The two rabbits that gave the lowest background by indirect immunofluorescence performed under different fixation methods were chosen for immunization. The first immune sera were tested by indirect immunofluorescence on HGPS fibroblasts; one serum gave no staining and one gave a promising signal. By bleed three, the rabbit that gave no signal remained negative. The rabbit with a positive signal remained positive, and the antibody titer had increased.

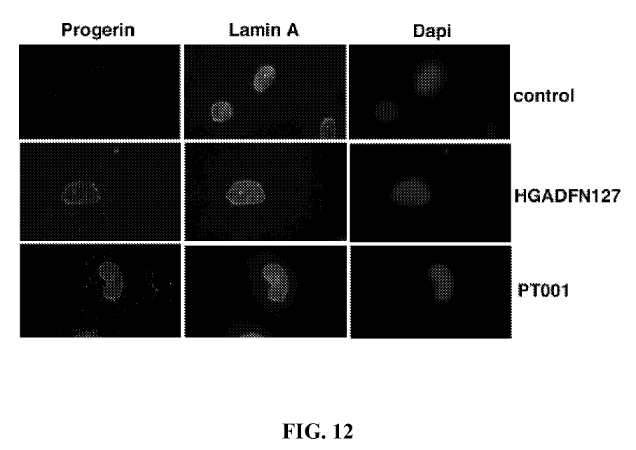

Progerin is expressed at very low level in early cellular passages below 10 passages: Anti-progerin serum was tested on celled in early cellular passages from passage number 6 for PT001 and from passage number 8 for HGADFN127 to passage 14 up to now. Surprisingly, only a small percentage of cells was positively labeled with anti-progerin antibody at passage 8. The signal was weak and was localized in the nucleus. After double immunofluorescence staining with anti-lamin A monoclonal antibody Jo14 and anti-progerin serum, it was observed that progerin was localized at the nuclear envelope periphery within the nuclear lamina (FIG. 12). In addition, progerin displayed a similar nuclear distribution as the wild-type lamin A in HGPS cells (FIG. 12). No staining was detected in control fibroblast with anti-progerin antibody (FIG. 12 panel control).

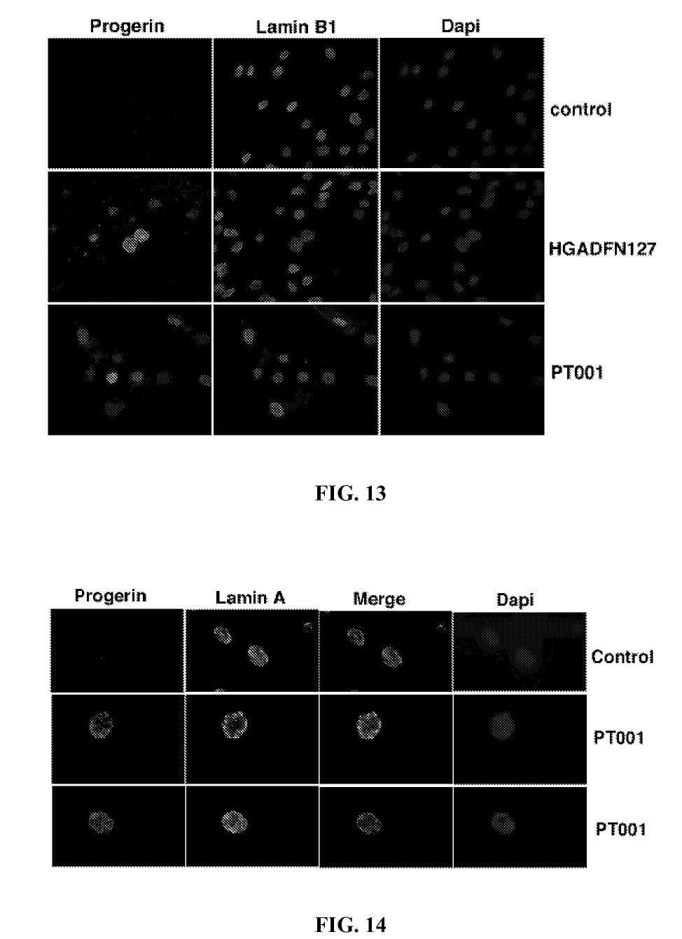

The weak signal obtained with anti-progerin antibody and the reduced number of positive cells lead us to believe that the antibody was not detecting progerin in some cells, which can occurs because the antibody epitope recognition on progerin was masked or because the level of progerin expression was to low. Immunofluorescence staining with anti-progerin antibody was performed on HGPS cells at each new cellular passage. The number of positively labeled HGPS cells that were observed with anti-progerin antibody staining increased with the cellular passage number and that the staining became stronger. In FIG. 13, a double staining with human anti-lamin B1 (Lassoued et al., 1992), and anti-progerin antibodies on HGPS cells at passage number 11 was performed. As shown in panels HGADFN127 and PT001, not all cells are labeled with anti-progerin antibody, as compared to the corresponding Dapi staining. Furthermore, the progerin signal is variable, in some cells it is faint, in others slightly stronger, and, in a few the signal was comparable to the signal obtained with anti-lamin B1 antibody (FIG. 13, panels lamin B1). These results indicate that progerin expression increased concomitantly with the increase passage numbers and that the population of HGPS cells in culture exhibit different levels of progerin expression. These results also indicate that anti-progerin antibody can detect progerin in situ, and cells negatively stained must not express progerin or express it at a very low level that is below detection with this method. Progerin localization in HGPS cells showed a similar intranuclear distribution as lamin B1 (FIG. 13, panels lamin B1), indicating that progerin is within the nuclear lamina and is colocalized with the filament network containing lamin B1. The Dapi staining does not reveal any dramatic chromatin rearrangement in HGPS cells expressing a detectable amount of progerin protein versus control fibroblasts (FIG. 13, panels Dapi). In HGPS cells as in control fibroblasts, the chromatin appeared homogeneous in the majority of the nuclei (FIG. 13). The majority of HGADFN127 and PT001 cells at passage number 11 do not exhibit severe nuclear abnormalities. However, it was observed that the nuclei were of different size and shape in comparison to control nuclei, which were more regular and ovoid (FIG. 13, panel Dapi).

Progerin accumulation in HGPS cells causes a dramatic rearrangement of the lamin network and the chromatin structure: Progerin accumulates in relation to the increase in population doublings and, by passage 14 the majority of HGPS cells in culture, express a detectable level of progerin, with most cells being positively labeled with anti-progerin antibody. The level of progerin expression is variable from cell to cell, which indicates that HGPS fibroblast cultures must contain different populations of cells that express varying levels of progerin. These results indicate that some cell populations can express the mutant lamin A (progerin) more efficiently than others.

At passage number 14, in addition to cells stained strongly with anti-progerin at passage 11 (FIG. 13), in nearly 4% of the cells there was a stronger accumulation of progerin. In most cells, progerin colocalized with the lamin A network at the nuclear periphery and the nuclear interior. Some cells exhibited thicker lamina staining with anti-progerin and anti-lamin A antibodies indicating that progerin accumulation induces thickening of the lamina. In a subpopulation of HGPS cells (0.5%), progerin accumulation caused a dramatic rearrangement of the lamin network (FIG. 14, panels PT001). In these cells, progerin induced the lamin filaments to form thick cable-like structures as well as the formation of lamin filament aggregates. Progerin signal was superimposible with the rearranged lamin A network in some of the aggregates; the signal with anti-progerin appeared stronger than the lamin A staining (FIG. 14, panels Merge). In cells containing lamin cable-like structures and aggregates, Dapi staining revealed compact areas of chromatin and the presence of holes in the chromatin distribution in the intra nuclear interior, indicating that the chromatin had also been reorganized (FIG. 14, panels Dapi). This indicates that progerin acts as a dominant negative mutant at high levels of expression by inducing rearrangement of the lamin network and chromatin redistribution.

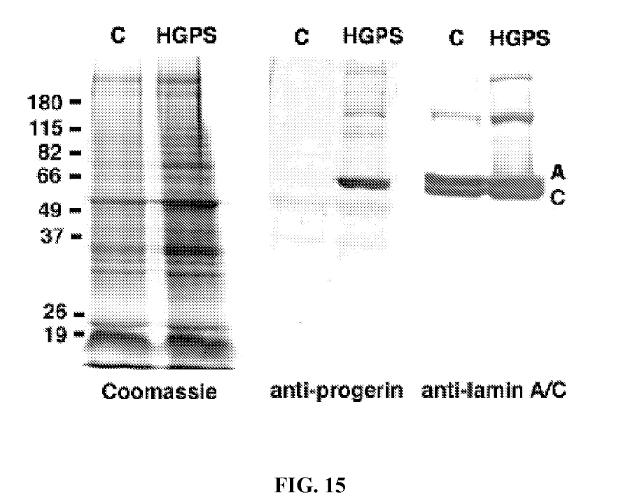

Detection of endogenous progerin expression by Western blot analysis: Total cell extracts from HGADFN127 and control fibroblasts at passage number 14, when most cells were positively labeled with anti-progerin antibody were used to prepare protein extracts. FIG. 15 shows a coomassie blue stained gel of extract control (C) and HGPS (HGADFN127). A replica of the gel was transferred on nitrocellulose and probed with anti-progerin antibody (1/2000) using the same method described previously (Djabali et al., 2001). The same western blot was washed and blocked in PBS buffer containing 5% non fat milk, 1% gelatin and 0.1% Tween-20 and re-probed with anti-lamin A/C (1/5000) (Chaudhary and Courvalin, 1993). In FIG. 15, anti-progerin antibody detects specifically one band migrating between lamin A and C. Thus, the polyclonal anti-progerin serum and antibody is specific to progerin by western blot analysis and that the signal is high at passage number 14, showing that this antibody is very useful in monitoring the expression levels of progerin in earlier cellular passages and will allow quantitative analysis in any of the experiments described herein, wherein quantitation of progerin is useful. The antibody of the invention will permit proteomic analysis as described herein.

In vivo localization of progerin expression in skin biopsy section derived from HGPS: Frozen skin sections derived from a skin biopsy of a subject with HGPS carrying LMNA G608G mutation—a skin biopsy sample from a 9 year-old donor were analyze by immunohistochemistry. 6 μm skin sections were fixed by methanol/acetone (IV/IV) at −20° C. for 10 minutes and washed in PBS, then blocked in PBS buffer containing 3% BSA, 10% normal goat serum and 0.3% NP40 for 1 hour. Slides were incubated with either rabbit polyclonal anti-progerin serum (dilution 1/400), the corresponding premium serum (PIM, 1/400), or with rabbit polyclonal anti-lamin A/C antibody (dilution 1/600) in blocking buffer for 1 hour. After 6 washes in blocking buffer, slides were incubated with goat anti-rabbit affinity purified IgG conjugated to Cy3 (dilution 1/400; from Molecular Probes). Slides were washed in blocking buffer 3 times and 3 times with PBS and mounted with Vectasheld mounting medium (Vector Inc.) One section was stained with hematoxylin and eosin for morphological comparison. Serial sections from the skin biopsy frozen in tissue freezing medium (Triangle Biomedical Science Inc) were used.

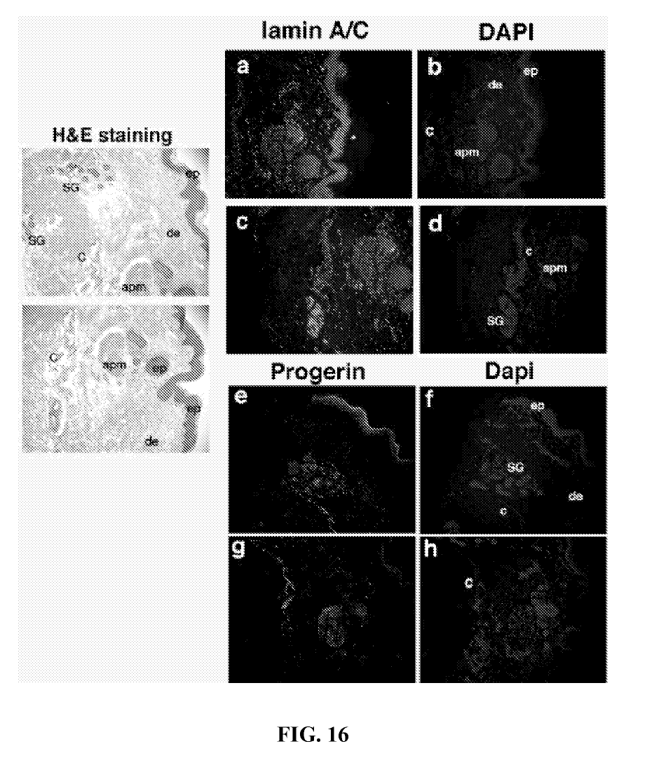

Progerin was expressed in a restricted subcellular populations: The skin sections, including the sections derived from HGPS subject as described supra, were analyzed under a Leica microscope and pictures were acquired at ten-fold magnification. FIG. 16 shows the hematoxylin and eosin staining of one section; two pictures from the section illustrate the different compartments: the epidermis and dermis. The epidermis is a keratinizing stratified squamous epithelium, mostly composed of keratinocytes. The epidermis continuously renews itself. The dermis supports the epidermis and is composed of the fibrous connective tissue components (collagen and elastic fibers) that surround the epidermal appendages (hair follicles, sebaceous glands, sweat glands). The dermis also contains blood vessels, nerves, as well as different types of cells, including mast cells, fibroblasts, myofibroblasts and macrophages. There is also smooth muscle tissue of the arrector pili muscle. FIG. 16, panel H&E corresponds to a hematoxylin and eosin staining of a skin section from the HGPS sample and shows the presence of a well organized epidermis, with the presence of a large blood vessel or capillary (C), sweat glands (SG), an arrector pili muscle (apm), and absence of hair follicle. The connective tissue is very dense and is detected as thick fibrous structures throughout the dermis. No subcutaneous fat tissue was detected in this sample. The next section was stained with anti-lamin A/C antibody (FIG. 16, Panels lamin A/C, a to d). Lamin A/C is detected in the nuclei of most cells from the epidermis and in the dermal compartments. The next section was immuno-labeled with anti-progerin antibody and shows a very restricted pattern of distribution. Positively labeled nuclei are localized within the blood vessel (C) and in some cells surrounding the sweat glands (SG) and the arrector pili muscle (apm). In the epidermis on this section, 6 keratino-cytes in total, which were positively stained with anti-progerin in the most upper layer of the epidermis were detected. The positive signal (FIG. 16, progerin, panel e) in the epidermis is due to the background staining of the cornified layer of the epidermis, also observed with the preimmune serum. The next section was incubated with the preimmune serum of the rabbit immunized with the progerin peptide and the background staining was extremely low, indicating that the signal obtained with anti-progerin antibody was specific.

In one aspect the invention provides a specific anti-progerin antibody that works by immunohistochemistry and biochemistry. This tool will allow specific examination of HPGS sections, including a methods to determine stages of progression of the disease. Since accumulation of progerin in the blood vessels can indicate already an advanced stage of the progression of the disease; monitoring the expression of progerin in vivo using skin biopsies from subjects of different ages can define stages of evolution of the disease, which will be very used for design of therapeutic treatments.

Methods to identify lamin A- and progerin-associated proteins: To test whether lamin A- and progerin-associated proteins can be isolated from HGPS fibroblasts nuclei preparations, a coimmunoprecipitation experiment using HGPS fibroblasts PT001 from passage number 12 (approximately 24 population doublings in the culture conditions described herein) was performed. The culture conditions were optimized for large-scale preparation of HGPS cells and a total cell pellet of 10⁷ cells was collected. In parallel the expression level of progerin and the proliferative index on cells from the same passage grown on glass coverslips were verified. By indirect immunofluorescence the expression level of progerin in fibroblasts PT001 was verified and a low level of progerin expression was observed in most of the nuclei identical to panels shown in FIG. 13. BrdU incorporation, for 1 hour at 37° C. in complete growth medium supplemented with 5 μg/ml BrdU (Sigma), was performed on another set of cells grown on glass coverslips and at the same passage number for control and PT001 fibroblasts to determine the proliferative index. A direct count of a 1000 cells was made for each cell type, and the percentage of BrdU positive cells was 53% for control fibroblasts and 46% for PT001, indicating only a small decrease in growth rate for PT001 at this cellular passage number 12. Communoprecipitation experiments were performed by methods described herein.

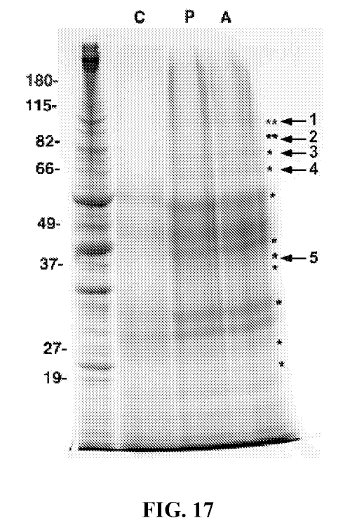

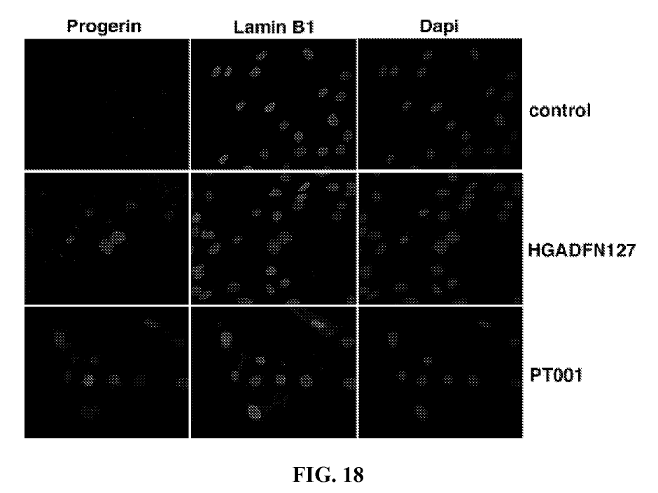

Communoprecipitation assay: Nuclei were isolated from 10⁷ PT001 cell pellet, and nuclear extract were either immunoprecipitated with mouse anti-human lamin A (Jol 4), or purified IgG from the rabbit polyclonal anti-progerin serum, or control rabbit IgG isolated from preimmune serum. Immunoprecipitated samples were resuspended in Laemmli buffer (Laemmli, 1970), electrophoresed on a large 10% SDS-PAGE gel, and stained with Coomassie blue as shown in FIG. 17. For control IgG (lane C), only a few bands were detect, indicating that the conditions for coimmunoprecipitation are clean. For anti-progerin IgG (lane P) and anti-lamin A monoclonal antibody (lane A), several major bands were detect, and most of them were visible on both lanes. Protein band at position marked number 2 points to a band weakly stained in the lamin A lane and not detectable in the progerin lane. The protein bands indicated (positions numbered 1 to 5) were manually excised for mass spectrometry analysis. Mass spectrometry was performed by a standard methods, as described herein.

This analysis identified the presence of lamin A and C in the complex. Actin and protein kinase C, which are two components that have been previously identified as lamin A interacting proteins were also identified (Zatrow M. S. et al., 2004). Furthermore, the presence of actin-associated proteins was observed, such as alpha-actinin, myosin, caldesmon, and plectin, which indicates their presence in the nuclear compartment and their possible structural role in the nuclear organization with the lamin network (Pederson T. and Aebi, 2003). Some proteins that are found in the cytoplasmic compartment such as vimentin can be selected as false positives; however, in this case, vimentin has been well documented as a lamin B interacting protein (Georgatos and Blobel, 1987; Papamarcaki et al., 1991). This experiment illustrates the use of this proteomic approach to isolate and identify lamin A partners. A rigorous and careful analysis of protein complexes isolated with anti-progerin and anti-lamin antibody will be done in parallel to control IgG in order to subtract any proteins that can be considered as false positive signals. Similar studies will be carried out on nuclear extracts from control dermal fibroblasts and will determine any significant changes in protein compositions between HGPS lamin complexes and control lamin complexes. This analysis indicates that protein complexes from progerin and lamin A can be isolated using this methodology. Better resolution of protein bands from the protein complexes can be achieved a using 2-dimentional gel separations of the protein samples. Moreover, by combining 2D gel and Ruby dye staining (fluorescent staining method, Biorad) the amount of each protein spot will be quantified and protein will be identified known methods in the art, including mass spectrometry as described herein.

TABLE 3
Proteins identified in the lamin A complexes isolated
from HGPS nuclear extracts with anti-lamin A antibody.

| | Number of peptides |
|---|---|
| Protein identified | identified |
| Myosin Heavy Chain type A | 70 |
| Lamin A/C | 33 |
| Alpha-actinin 1 | 27 |
| Actin | 26 |
| Caldesmon | 22 |
| Alpha-actinin 4 | 12 |
| Plectin | 10 |
| heterogeneous nuclear ribonucleprotein | 8 |
| M | |
| Vimentin | 5 |
| Protein Kinase C | 4 |
| polymerase I-transcription release factor | 3 |
| DEAD-box protein 3 | 3 |
| RNA helicase A | 3 |

Dissecting the molecular mechanisms of alterations induced by the mutant lamin A G608G on nuclear functions in HGPS cells: Progerin's mode of expression is interesting because at early cellular passages it is not detected, and it appears to accumulate in a cellular-age dependent manner. Moreover, cell proliferation appears to decrease concomitantly with the accumulation of progerin, cells become apoptotic or enter a senescence stage. This observation indicates that progerin has a negative effect on the cell cycle progression and somehow mimics the effect of a tumor suppressor. The rate of growth will be followed according to the level of progerin expression and it will be determined at which level, progerin causes the deregulation of growth control and define which signaling pathways and which molecular changes within the nucleus are causing such an effect.

The anti-progerin antibody can be used in methods and assays to determine: at which cellular passage stage is progerin detected; how does progerin assemble, and where does it accumulate in the nuclear compartment; how does progerin behave during mitosis; whether progerin induce changes on the nuclear repartition and composition of known lamin A-interacting partners; whether the distribution of the nuclear pore and whether nuclear transport is affected by progerin; the proliferation index of HGPS cultures according to the level of progerin expression and determine when progerin accumulation has a deleterious effect on growth; whether cells expressing high level of progerin enter senescence stage.

Dermal fibroblast cells were obtained from Progeria Research Foundation three primary cultures dermal fibroblast derived from HGPS patients carrying the LMNA mutation G608: HGADFN001 cells were derived from a 10 year old patient; HGADFN003 from 2 year old; and HGAFN127 from 3-year-and-9-month old. Furthermore, fibroblast cells were derived from the newly identified HGPS case PT001 described in preliminary results.

Indirect immunofluorescence analysis: Primary cultures of dermal fibroblasts from patients and control can be grown on glass coverslips, and processed for indirect immunofluorescence as described previously (Djabali et al., 2001; Djabali et al., 1997). Rabbit antibodies directed against A-type and B-type lamins have been reported previously (Cance et al., 1992; Chaudhary and Courvalin, 1993). Monoclonal antibodies anti-emerin 4G5 (Novocastra), anti-nucleoporin Nup153 clone 414 (Covance), anti-pRb G3-245 (Pharmingen), anti-PKC□ (Stressgen), anti-lamin A Jo14 (Serotec) will be purchased. Anti-Lap2a antibodies were reported previously (Dechat et al., 2000). The secondary antibodies will be purchased from either Molecular Probes or Jackson ImmunoResearch laboratories). DNA will be stained by DAPI (Sigma). Double immunofluorescence will be performed to monitor the repartition of the different nuclear components in comparison the progerin signal. Confocal microscopy analysis will be performed by methods known in the art.

Nuclear transport: Clustering of the nuclear pores was detected in late cellular passages in HGADFN003 (Goldman et al., 2004). Based on this observation, this rearrangement may alter nuclear transport. To determine this, the nucleocytoplasmic shuttling of Smad2 and 3 will be followed in HGPS cells in late passage, when progerin accumulates. Upon transforming growth factor beta (TGF-beta) stimulation, one of the signaling pathways known to operate downstream TGF-beta is the Smad pathway (Massague et al., 2000). In unstimulated cells Smad2 and 3 are predominantly cytoplasmic: upon TGF-beta stimulation they accumulate in the nucleus. HGPS fibroblasts grown on coverslips will be treated with TGF-β1 (Pepro Tech) at a final concentration of 2 ng/ml growth medium for 1 hour to induceSmad2/3 nuclear translocation. Cells will be labeled by indirect immunofluorescence with anti-Smad2/3 monoclonal antibody (Transduction laboratories) and anti-progerin antibody. The localization of Smad2/3 will be monitored in cells expressing high level of progerin.

Quantification of the proliferative index: Four biological markers of cell proliferation can be followed: Bromodeoxyuridine (BrdU) incorporation, histone H3 phosphorylation, Ki-67 protein expression, and retinoblastoma protein pRB phosphorylation, by indirect immunofluorescence detection, with specific antibodies against the corresponding proteins that are all commercially available. The 4 markers represent well-characterized biomarkers specific for both total proliferative cells (Ki67, phospho pRb, and BrdU-4 h incorporation) and for specific cellcycle population (phospho-Histone H3 for M phase, BrdU 30 min incorporation for S phase) (Gasparri et al., 2004). This study will define at which cellular-age and level of progerin the cell cycle progression is inhibited.

Characterization of the heat stress response of HGPS fibroblasts: A delayed response to stress was observed in HGPS PT001 (FIG. 11). The stress response of other HGPS primary cell lines from the Progeria Research Foundation will be analyzed to determine whether it is a common defect of the HGPS fibroblasts. Cells exposed to heat shock respond to stress by increasing the expression of specific set of proteins referred to as heat shock proteins (HSPs), (Jolly and Morimoto, 1999; Morimoto, 1998). The effect of heat stress on protein expression levels and intracellular localization of two small HSPs, αBcrystallin and HSP27, as well as HSP70, a member of a different class of heat shock proteins that are known to interact with intermediate filament proteins, will be examined in order to unravel the cause of the defective response to stress observed in HGPS fibroblasts.

HGPS fibroblasts and control fibroblasts will be either grown under normal conditions or submitted to heat shock (30 min at 45° C.) and returned to 37° C. for 2 hours, 24 hours and 48 hours to recover from stress. Indirect immunofluorescence analysis will be performed using commercially available antibodies directed against anti-αB-crystallin (Santa Cruz) or Hsp27 (Santa Cruz) and Hsp70 constitutive and inducible Hsp70 (Stressgen). In parallel, HSP protein levels will be determined by Western blot analysis. This analysis is will determine: (1) whether HGPS fibroblasts express the same levels of HSP proteins, (2) the kinetics of their appearance and accumulation, (3) their intracellular localization and (4) their potential recruitment on the lamin network.

Protein kinase Cα (PKCα) is a direct binding partner of lamin A and interacts with lamin A carboxyl-terminal region within amino acids residue 500 to 664 (Martelli et al., 2002). This domain overlaps the truncated region in progerin indicating a possible defect in progerin association with PKCα. Moreover, PKCα is involved in stress signaling pathways (Coaxum et al., 2003; Joyeux et al., 1997); analysis of the stress response in HGPS fibroblasts provides a physiological assay to test lamin A-PKCα interaction. Under control (no heat shock) and heat shocked conditions, the cellular distribution of PKCα in HGPS cells will be investigated, by immunofluorescence detection and Western Blot analysis to follow the repartition between the cytoplasmic and nuclear compartment using anti-PKCα antibody (Stressgen). Colocalization of PKCα with lamin A and progerin will be analyzed by confocal microscopy.

Senescence analysis of HGPS dermal fibroblasts: Determining whether HGPS fibroblasts with high progerin content are senescent will provide the first indication of how tissues can be affected in subjects with HGPS. The tissues most affected in subjects with HGPS are those that have renewal potency: skin, bone, and muscle. Thus, cells in renewable tissues may deplete their replicative potential because of progerin accumulation. If this is the case, senescent cells will accumulate in vivo, and will contribute to an age-related pathology observed in HGPS patients. To identify senescent cells in HGPS fibroblast cultures, a beta-galactosidase staining at pH6.0 will be performed (Dimiri et al., 1995). Beta-galactosidase activity at pH6 has been identified as a biomarker for senescent cells; at this pH range quiescent cells are not labeled (Dimiri et al., 1995).

Senescence-associated β-Galactosidase staining: HGPS cells grown on coverslips will be fixed in 3% formaldehyde, then washed and incubated overnight in fresh staining solution: 1 mg of 5-btomo-4-chloro3-indolyl b-D-galactoside (X-Gal) per ml (from stock 20 mg of dimethyldormamide per ml), 40 mM citric acid/sodium phosphate, pH6, 0.5 mM potassium ferrocyanide/5 mM potassium ferricyanite/150 mM NaCl/2 mM MgCl2 (Dimiri et al., 1995), and processed for indirect immunofluorescence with anti-progerin antibody. beta-Galactosidase positive cells will be scored in comparison to progerin signal, using light and fluorescent microscopy respectively. If a direct correlation is observed that such senescence positive cells are the ones with high content of progerin, a β-Galactosidase staining on skin sections derived from HGPS skin biopsies will be performed to determine if cells from the blood vessels are Galactosidase positive (FIG. 16, panel g). These experiments will be informative in understanding the mechanisms leading to atherosclerosis, a key component in the pathology of HGPS.

Retinoblastoma protein (pRb) status in HGPS cells: pRb plays a critical role in the regulation of cell cycle progression (Weinberg, 1995) and cellular senescence (Mosachi et al., 2003). Furthermore, pRb was reported to interact with lamin A (Ozaki et al., 1994). The intracellular distribution of pRb will be monitored in a cellular age-dependent manner by indirect immunofluorescence studies (anti-pRb, Pharmingen); and it will be determined by Western blot analysis whether pRb expression is differently regulated in proliferating versus non-proliferating HGPS fibroblasts.

Reversing the HGPS cellular phenotype by inhibition of progerin expression with RNAi: The reading frame of progerin from amino acid 601 to 611 is shown below.

| | |
|---|---|
| nt 1801 - GGA GCC CAG AGC CCC CAG AAC TGC AGC ATC ATG | (SEQ ID NO: 51) |
| aa 601 - S G S G A Q S P Q N C - aa 611 | (SEQ ID NO: 52) |

Highlighted in bold are—according to the wild type lamin A sequence—amino acids S656 and Q606 that are adjacently linked to each other in progerin. To target specific degradation of progerin mRNA small interfering RNA (siRNA) duplexes within the region described herein will be designed. Sequences of the type AA(N19)UU (N, any nucleotide) will be selected from the open reading frame of the targeted progerin mRNA corresponding to the region from above. The different siRNA sequences will be submitted to a BLAST search against the human genome sequence to ensure that no other of the gene of the human genome is targeted. Based on these first criteria the 21-nt siRNA options will be selected and will be purchased from Dharmacon (Lafayette, Colo.). Other regions of the lamin A cDNA cannot and will not be used since it is not desirable to inhibit the expression of the wild type lamin A.

siRNA transfection: HGPS cells will be transfected with 1 μg siRNA duplex per well, using oligofectamine according to the manufacturer's instructions (Invitrogen). Cells will be fixed 60 hours post-transfection, and immunofluorescence will be performed using anti-progerin antibody. If specific inhibition of progerin with one of the siRNA tested is identified, this sequence will be tested for further characterization of the possible reversion of progerin effects on the nuclear envelope, lamina, and chromatin, as well as on the cell cycle, as described herein, and determine at which level of progerin expression possible reversion can occur.

Identification of lamin A-interacting protein(s) and characterization which of these protein(s) no longer interact with proverin: Methods described herein combine cell biology and biochemical approaches for analyzing the function of progerin. Lamin A plays a central role in many cellular functions, most likely through specific interactions with other nuclear proteins to maintain the integrity of the nuclear scaffold, to facilitate chromatin organization, and to assist gene expression. HGPS fibroblasts exhibit altered nuclear shape indicative of a disorganization in the nuclear envelope protein composition or in the nuclear lamina that is likely the result of some defective protein-protein interactions. HGPS fibroblasts show altered chromatin organization that also indicates defective protein-chromatin interactions. Those defects result from the expression of the mutant lamin A progerin. As such identifying the protein-protein interactions that are impaired by progerin in HGPS model system will determine the molecular mechanisms that progerin utilizes to alter the organization and functions of the nucleus. In addition these experiments will provide answers on the functional role of the lamin A1.

Lamin A- and Progerin-Associated Proteins Isolated from HGPS Dermal Fibroblasts

Antibodies and HGPS cells: Communoprecipitation assays (COIP) will be performed using nuclear extracts from primary cultures of control (such as normal) and HGPS fibroblasts. For these experiment, a mouse monoclonal anti-progerin antibody can be generated. As described herein a rabbit polyclonal antibody was generated using the progerin peptide as antigen. The same peptide sequence can be synthesized and used to generate, by methods known in the art, a monoclonal antibody, for example but not limited to a mouse monoclonal antibody, to ensure purity, specificity, and concentration for CoIP. The monoclonal antibody production will be custom made. In one embodiment, the monoclonal antibody is generated by fusion of splenic B-cells, from the rabbit source of the polyclonal antibody, to myeloma cell line to generate hybridoma fusions, which produce specific monoclonal antibody against progerin. The supernatant from two hybridoma cell lines is analyzed to determine whether the hybridoma produces anti-progerin antibody. The supernatant from two hybridoma cell lines, after a third subcloning (purification) step, have been analyzed, and determined to produce monoclonal antibodies against progerin. The monoclonal antibody will be fully characterized prior to use. Alternatively, coimmunoprecipitation can be carried out with the rabbit polyclonal anti-progerin antibody after affinity purification over the progerin peptide. For lamin A Co-immunoprecipitation assays, the mouse anti-human lamin A clone Jo14 will be purchased from (Serotec).

Communoprecipitation: HGPS fibroblast cultures will be expanded as described herein to reach the cellular passage (which will be cell line-dependent) where progerin protein is expressed at a detectable level, so that immunoprecipitation of progerin from nuclear extract can be performed. This study will require a range of 10⁷ cells for nuclei isolation and CoIP. Prior to cell collection, progerin expression will be monitored by indirect immunofluorescence and Western blot analysis using anti-progerin antibody. 10⁷ cells will be harvested and nuclei will be isolated and washed with cytoskeletal extraction buffer (10 mM PIPES (pH 6.8), 300 mM sucrose, 100 mM NaCl, 3 mM MgCl2, 1 mM EGTA, and 0.5% Triton X-100). Subsequently, the nuclei preparation will be extracted in KHM buffer (50 mM Hepes, pH 7.4; 78 mM KcL, 10 mM EGTA, 8.4 mMCaCl2, 4 mM Mgc12) containing 1% triton X 100 1 mMDTT 500 μg/ml DNAse, 200 μg/ml RNAse A, protease inhibitor, PMSF 1 mM incubated at RT for 10 min, and sonicated briefly. To 800 μl of nuclear lysate, 25 μl protein G-Sepharose beads (Sigma) will be added, centrifuged for 3 min at 1000 rpm (Heraeus microfuge) and the supernatant will be mixed with 50 μl of Protein G-Sepharose beads that would have been preincubated with either mouse anti-lamin A Jo14, or mouse anti-progerin antibodies, or monoclonal progerin antibody, or control (e.g. mouse) IgG. Incubation will be performed by end-over-end rotation; the beads will be pelleted through 30% sucrose at 1000 rpm for 3 min at 4° C. Beads will be further washed 5 times in KHM buffer. Immunoprecipitated samples will be separated on 2-D gel electrophoresis using Protean IEF Cell from BioRad, and protein identification will be performed by mass spectrometry analysis as described herein.

Lamin A- and Progerin-Associated Proteins from Stable Endothelial Cell Lines

Cell culture transfection, and generation of stable cell lines: Based on the findings that progerin was primarily expressed in endothelial cells on HGPS skin Biopsy section (FIG. 10), human endothelial cell line ECV304 (American Type Culture Collection) will be used. ECV304 cells, a spontaneously immortalized cell line of human umbilical vein origin (Takahashi et al., 1990) have been shown to retain hormonal/growth activities in culture and to respond to their extracellular environment by initiating cellular differentiation (Wang and Passaniti, 1999), and as such will provide a good model system for the analysis described herein. ECV304 cells are grown in Dulbecco's modified Eagle's medium containing 2 mM L-glutamine, 10% fetal calf serum and 100 units/ml penicillin/streptomycin (Invitrogen). Full-length cDNA for lamin A and progerin have been generated as described herein. A His6 tag will be added at the 5′ end of the ATG of lamin A and progerin cDNA by PCR with specific primers containing the His6 tag and restriction site to allow direct subcloning into pIRESpuro2 (Clontech). Stable endothelial-His6-lamin A and endothelial-His6-progerin cell lines will be made by transfecting endothelial cells with 15 μg of pIRESpuro2-His6-lamin A or pIRESpuro2-His6-progerin plasmid using FuGENE 6 reagent (Roche Applied Science) according to the manufacturer's instructions. Selection will be carried out in growth medium supplemented with puromycin (5 μM). Stable cell lines will be characterized by indirect immunofluorescence studies.

Purification of lamin A- and progerin-associated proteins: Proteins associated to His 6-Lamin A or His6-progerin will be purified using isolated nuclei preparations. 10⁷ endothelial cells (either endothelial-His6-lamin A, endothelial-His6-progerin, or control parental endothelial cells) will be isolated by trypsinization and washed twice with ice-cold phosphate-buffered saline. Nuclei will be isolated and washed with cytoskeletal extraction buffer (10 mM PIPES (pH 6.8), 300 mM sucrose, 100 mM NaCl, 3 mM MgCl₂, 1 mM EGTA, and 0.5% Triton X-100). Subsequently, nuclei will be extracted in KHM buffer (50 mM Hepes (pH 7.4), 78 mM KCl, 10 mM EGTA, 8.4 mM CaCl₂, 4 mM Mgcl₂) containing 1% triton X 100, 1 mM DTT, 500 μg/ml DNAse, 200 μg/ml RNAse A, protease inhibitors, PMSF 1 mM. Lysate will be incubated for 10 min at RT, 30 min on ice, and then briefly sonicated. His6-progerin or His6-lamin A complexes will be enriched on Ni²⁺-nitrilotriacetic acid-agarose beads (QIAGEN Inc.). Beads will be successively washed with 20-column volumes KHM buffer, then in buffer KHM lacking Triton X-100. Protein complexes will be eluted in 0.2 column volumes of KHM buffer containing 200 mM imidazole.

Isoelectric focusing Using Protean IEF cell: Fractions eluted from the affinity purifications will be precipitated using ready prep 2-D cleanup kit (BioRad). Pellets will be resuspended in equal volume of rehydratation buffer (8 M urea, 4% CHAPS, 60 mM DTT, 10% glycerol, and 1% ampholites pH 3-10). Protein samples will be loaded on immobilized pH gradient strips from pH 3-10 using in gel rehydratation for 15 hr at 20° C. After this step, the IEF strips will be submitted to isoeletric focusing on Protean IEF cell according to manufacturer (BioRad).

2-Dimensional gel analysis: Analytical gels will be stained with SYPRO Ruby (BioRad) or transferred to nitrocellulose for western blot analysis. Preparative gels will be stained with coomassie G250. The level of protein expression will be determined on SYPRO Ruby stained gels using VersaDoc Model 3000 imaging system (BioRad). One image analysis software that will be used to acquire and store experimental image data, to detect, and quantify the protein spots, and to compare different HGPS 2D gels to control gels is PDQuest software from Biorad. This program is very valuable as it offers the possibility to make a subtractive comparison of different gel series. A standard gel of each CoIP preparation series can be created such that it will become the reference 2D gel. Each 2D gel reference can be automatically compared to every experimental assay. In addition, 2-D gel protein data bank for lamin A-interacting proteins that can be established and accessed by others working on laminopathies.

Identification and characterization of the protein complexes: Assays described herein can be used to determine what the lamin A- and progerin-associated protein complexes are. Protein spots that are present in the control samples will be subtracted. The remaining protein spots will be quantified, and protein identification will be carried by mass spectrometry methods known in the art. The protein identification will be done by excision of the protein spots from coomassie stained preparative gels. The gel pieces are washed twice in 25 mM NH₄HCO₃, 50% acetonitrile, spun dry and in gel trypsin digested in 10 ng/μl trypsin in 25 mM NH₄HCO₃ for 16 hours at 37° C. Peptides will be extracted from the gel with 50% (V/V) acetonitrile, 1% (V/V) TFA solution. Matrix assisted laser desorbtion ionization-time of light (MALDI-TOF) mass spectrometry acquisition will be performed on a TofSpec 2E mass spectrometer set to reflectron mode. Known trypsin cleavage peptide masses will be used for a 2-point internal calibration for each spectrum. Monoisotopic peptide masses will be searched against the theoretical peptide masses of human proteins in the Swiss-Prot and TrEMBL protein databases using the MassLynx search program. A minimum of 4 peptides is required for a positive identification.

Proteins identified by CoIP from HGPS and/or endothelial cells will be compared, and a subtractive analysis of the data will be performed between lamin A and progerin protein complexes to identify components that exhibit detectable changes in the progerin samples. Analysis can determine whether lamin A- and progerin-associated proteins that are common between both cellular models and the ones specific to endothelial cells. Furthermore, proteins showing a significant difference between lamin A and progerin complexes will be prioritized in studying their relationship with progerin by immunocytochemistry and biochemical studies.

The interacting proteins will be characterized as follows: (1) Antibodies commercially available will be purchased and immunohistochemistry will be carried out to see whether or not a protein is localized in the nucleus and is colocalized with lamin A and progerin by confocal microscopy. (2) Image clones or EST clones of the protein, if available, will be purchased; if not, the peptide sequences obtained from mass spectrometry analysis can be blasted against available sequence databases. Oligonucleotide primers will be designed according to the human genome sequence data obtained via BLAT search and used to amplify by RT-PCR the corresponding cDNA from the control fibroblast or endothelial cell mRNA preparations. Interaction studies with lamin A and progerin will be performed by GST-pull down to test direct interaction and by co-immunoprecipitation assays for direct and indirect interactions.

GST affinity binding assay: Expression and purification of GST-tagged interacting proteins will be performed as follows: cDNAs will be subcloned in PGEX6P1 vector and expressed in BL21 bacteria strain. GST fusion proteins will be purified on glutathione-Sepharose 4B (Pharmacia) column according to standard protocol and as previously described (Djabali et al., 1997). The GST binding assay will be performed by using 20 □l of GST beads coated with GST-protein and mixed with equal amount of His-C-terminal lamin A fragment or progerin fragment for 1 hr at 4° C. The beads will be collected and washed in HBB (40 mM Hepes, 75 mM KCl, 0.5 mM EDTA, 5 mM MgCl₂, 1 mM DTT, 1 mM PMSF, 0.5 mg/ml BSA, 0.05% NP40). The bound protein will be eluted in sample buffer and analyzed by SDS-PAGE and Western blot analysis.

Reverse-immunoprecipitation assay: New cDNAs will be cloned into PSVK3 plasmids (Invitrogen) and contain a Flag tag epitope. HGPS fibroblast cells will be transfected with PSVK3-Flag-new-cDNA construct. Endothelial cells will be cotransfected using 3 μg of pEGFP-progerin construct together with 3 ug of PSVK3-Flag-new cDNA per 10-cm dishes. 48 hr after transfection, cells will be harvested in PBS, and homogenized in immunoprecipitation buffer (IP) (50 mM Tris-HCl pH 7.4, 150 mM NaCl, 1% NP-40, 10% glycerol and protease inhibitors) at 4° C. The extracts will be incubated 2 hr on ice and centrifuged at 15,000 g for 10 min. The supernatants will be incubated overnight at 4° C. with anti-Flag antibody (Sigma). The samples will be further incubated for 3 hr with protein G sepharose (Sigma). The beads will be washed first with IP buffer containing 0, 2, or 4 M urea to test the stringency of interaction, followed by two washes in IP buffer alone. Immunoprecipitates will be fractionated by SDS-PAGE and analyzed by Western blotting. Western analysis will be performed with anti-Flag monoclonal antibody (Sigma).

Some lamin A interacting partners have already been identified (FIG. 2) and will serve as positive controls in validating these methods. The assays described herein seek to identify new components that are unknown, and as such, their levels of expression will have to be determined. The assays described herein can be used to identify direct interacting components and indirect partners. A complementary project using a micro-arrays analysis can be undertaken to identify any difference in mRNA expression profile between progeria cells versus control cells. Changes in mRNA levels for nuclear factors will be investigated further to define their relationship with lamin A and progerin.

Determining common cellular and molecular mechanisms underlying Atypical and Adult Progeroid Syndromes in comparison to HGPS: The 50 amino acid deletion in the carboxyl-terminal lamin A in HGPS is responsible for this premature aging phenotype by disrupting the lamin organization through disrupting some protein-protein interactions that are important for the integrity of the nuclear compartment. Table 1 is a summary of other lamin A mutation causing other age-related disorders such as Atypic progeroid and Adult progeroid disorders. Mutations in lamin A that are overlapping with lamin A mutation in HGPS will share common cellular and molecular mechanisms to elicit premature aging phenotype. To analyze this, two lamin A mutations will be investigated: LMNA R644C responsible for atypical progeroid syndrome (Csoka et al., 2004), and LMNA T623S responsible for adult progeroid disease (Fukuchi et al., 2004). It can be determined whether those mutants exhibit the same nuclear alterations, and whether their nuclear accumulation also causes disorganization of the lamina and inhibits cell-cycle progression. Protein complexes isolated with those two progeroid mutant lamin A may also alter the same protein-protein interactions as the one disrupted by the presence of progerin. Altogether those findings will provide a framework of the molecular mechanisms that are sensitive to lamin A mutations and as such can lead to methods of studying normal aging mechanisms.

Analysis of lamin A R644C: Primary fibroblasts harboring LMNA R644C corresponding to cell line AG00989 will be purchased from Coriell Cell Repositories and have been described previously (Csoka et al., 2004). The cDNA of lamin A R644C will be amplified by RT-PCR from total RNA preparations derived from AG00989 fibroblast cultures using specific primers containing restriction sites for direct subcloning into PEGFP-C1 plasmid. The final clones will be verified by direct DNA sequencing.

Primary fibroblast cultures AG00989 will be characterized by the assays and methods as described herein. The lamin A mutant R644C is predicted to be prenylated but not further processed because the missense mutation R644C alters the recognition site of the endoproteolysis enzyme zmspte24 (Corrigan et al., 2004). The distribution of lamin A will be analyzed in early and late passage cultures to determine whether in late population doublings the mutant lamin A increase the thickness of the lamina. Monitoring and analysis can be done by western blot analysis, using monoclonal anti-lamin A antibody (Jol 4) and rabbit polyclonal anti-lamin A./C, the appearance of a band corresponding to lamin A R644C that smigrates higher than the wild type lamin A because it is not processed. The growth rate of AG00989 cells will be defined in comparison to control fibroblasts cultures, by following different markers of proliferation. The population doubling will be determined when cells will start to show signs of growth retardation and compare them with HGPS cells.

Communoprecipitation and mass spectrometry analysis: Nuclei extract from AG00989 cells, at population doublings showing a detectable signal of the mutant lamin A by Western blot analysis, will be isolated for coimmunoprecipitation assays using the monoclonal anti-lamin A antibody (Jo14) as described herein. Data obtained with this mutant lamin A will be compared to data obtained with HGPS mutation.

Analysis of lamin A T623S: LMNA T623S was identified in a Japanese subject with adult HGPS who lived up to 45 years old (Fukuchi et al., 2004). This mutation creates a cryptic splice site producing a 35 amino acid truncation within the carboxyl-terminal tail domain overlapping with the deleted region in progerin.

The analysis of the LMNA T623S mutant will be carried out using a stable lymphoblastoid cell line established from peripheral blood sample from the patient using Epstein-Barr virus, as well as vector, pSP72, encoding the mutant lamin A cDNA from exon 9 through exon 12 (Fukuchi et al., 2004).

The full-length cDNA encoding the mutant lamin A T623S will be generated as follows: the cDNA insert will be excised with restriction enzymes BsiWI and EcoRI. PEGFP-C1 lamin A will be digested with the same enzymes, and the band corresponding to the open plasmid will be purified from the agarose gel. The insert containing the mutant lamin A fragment, will be similarly purified and ligated into the digested PEGFP-C₁. PEGFP-C1 lamin A T623S positive clones will be verified by direct DNA sequencing and comparison to the wild type lamin A reference sequence. This construct will be transfected into control fibroblast to examine the distribution of the mutant lamin and analyze the nuclear alteration such as lamin network disruption and chromatin reorganization in cells expressing lamin A T623S. Those nuclear alterations will be compared to the ones observed in HGPS fibroblasts. For coimmunoprecipitation and mass spectrometry analysis, any suitable method including the two methods described herein can be used. By indirect immunofluorescence and Western blot analysis, the expression levels of the mutant lamin A in the stable lymphoblast cell line will be tested. As show in the Fukuchi et al., report, a detectable amount of mutant lamin A is observed on a Western blot probed with anti-lamin A/C antibody. Nuclear extract will be prepared from the lymphoblast cell line, and the coimmunoprecipitation will be done with mouse monoclonal anti-lamin A (Jo14). Protein complexes will be resolved on 2D-gels and analyzed as described herein. In addition, lamin A T623S cDNA will be subcloned into pIRESpuro2, and a His6 tag will be add at the 5′ end of the ATG of the mutant lamin A. ECV304 endothelial cells will be transfected with His6-lamin AT623S pIRESpuro2, and stable clones will be selected and analyzed by indirect immunofluorescence and Western blot analysis to monitor the expression levels of the mutant lamin A. Nuclear extract will be prepared, and His6-lamin A T623S-associated proteins will be affinity purified on Ni²⁺-nitrilotriacetic acid-agarose beads and analyzed further as described herein. Protein complexes identified by both methods will be compared and analyzed together with the data obtained with HGPS cells.

The methods described herein will analyze the proteins associated with lamin A R644C and lamin A T623S. The protein reference list of each mutant will be analyzed in comparison to the lamin A G608G and wild type lamin A complexes. Differences between HGPS and Atypical and/or Adult progeroid models can indicate why subjects with these mutations live longer than subjects carrying the HGPS mutation (G608G) and identify protein-protein alterations that are responsible for the premature aging. Those components will be further analyzed to determine their mechanism of action in the nucleus and their effect on nuclear architecture, chromatin organization, and cell cycle progression as described herein.

Primary dermal fibroblast R644C are available and will allow for similar morphological analysis as in HGPS fibroblasts. For lamin A T623S, there is no dermal fibroblast culture available, and as a result, analysis of that mutant lamin A can only be performed on the stable lymphoblast cell line or on fibroblast cells transiently transfected with this mutant lamin A. This analysis will determine the analogous effect on the nuclear compartment of this mutant (T623S) to that of progerin.

Establishing a skin tissue bank from adult and elderly individuals: Skin samples were collected and annotated as shown in Table 4, to establish a tissue bank. For some of the samples, primary cultures have been established as indicated. Morphological analysis of the skin sections from the 20 specimens was conducted after Hematoxylin and Eosin staining. Analyses of elderly skin show a reduced thickness of the epidermis, reduced cellularity in the dermis and presence of disorganized connective tissue. Detailed morphological analysis will be performed as the number of samples increases and a higher number of specimens derived from same body sites will be available for comparison.

TABLE 4
Skin tissue bank

| | | | | | | Frozen | Box | | |
|---|---|---|---|---|---|---|---|---|---|
| Sample | Species | Gender | Age | Type | Body site | block | (80) | Slides | Cultures |
| D001 | Human | male | 90 | Skin | L temple | 2 | DR-1 | 8 | |
| D002 | Human | male | 74 | Skin | Nose | 2 | DR-1 | 8 | |
| D003 | Human | female | 74 | Skin | L Cheek | 3 | DR-1 | 7 | |
| D004 | Human | female | 85 | Skin | L scalp/forehea | 1 | DR-1 | 8 | |
| D005 | Human | male | 83 | Skin | L cheek | 2 | DR-1 | 6 | |
| D006 | Human | male | 45 | Skin | Scalp | 1 | DR-1 | 8 | |
| D007 | Human | female | 68 | Skin | L cheek | 1 | DR-1 | 7 | |
| D008 | Human | Male | 79 | Skin | L leg | 1 | DR-1 | 8 | |
| D009 | Human | female | 66 | Skin | Lip | 1 | DR-1 | 8 | |
| D010 | Human | male | 52 | Skin | Cheek | 1 | DR-1 | 8 | |
| D011 | Human | male | 76 | Skin | L leg | 2 | DR-1 | 6 | |
| D012 | Human | female | 59 | Skin | Scalp | 2 | DR-1 | 10 | |
| D013 | Human | male | 55 | Skin | Forehead | 1 | DR-1 | 8 | |
| D014 | Human | male | 86 | Skin | Nose | 1 | DR-1 | 8 | |
| D015 | Human | male | 64 | Skin | R. ear | 2 | DR-1 | 8 | |
| D016 | Human | female | 85 | Skin | Chest | 2 | DR-1 | 8 | |
| D017 | Human | female | 85 | Skin | Forehead | 2 | DR-1 | 6 | |
| D018 | Human | female | 84 | Skin | Chest | 1 | DR-1 | 8 | F, K |
| D019 | Human | male | 58 | Skin | R scalp | 1 | DR-1 | 8 | F |
| D020 | Human | female | 93 | Skin | forehead | 2 | DR-1 | 10 | F |

F—fibroblast culture;
K—keratinocyte culture

Determining the effect of the mutant lamin A G608G on nuclear functions in HGPS cells and skin: Endogenous progerin is characterized in primary dermal fibroblast cultures derived from subject with HGPS. A specific anti-lamin A G608G antibody was generated as described herein. The immune sera, denoted anti-LMNA G608G, were tested on four different fibroblast cultures derived from subjects with HGPS, obtained from Progeria Research Foundation. A small percentage of cells were positively labeled with anti-LMNA G608G antibody at early PPDs; the number of nuclei positively stained with anti-LMNA G608G increased with the number of cellular generations in vitro.

Functional alterations induced by progerin nuclear accumulation: Primary cultured cells undergoing cellular senescence in vitro express increased beta-galactosidase (β-gal) activity when assayed at pH 6. Indeed, when senescence β-gal positive cells were scored at late PPDs in HGPS cells, it was observed that the number of senescent HGPS cells increased with time in vitro. These results indicate that senescent cells accumulated prematurely in HGPS fibroblast cultures (McClintock et al., 2006).

To gain insight on the cellular effect of the mutant LMNA G608G in HGPS fibroblasts, cellular migration, which is an important function in a variety of physiological aspects, was examined. A migration assay was used to examine inherent motility and directional migration of fibroblasts in response to a chemotatic stimulus. It was observed that HGPS cells with high levels in LMNA G608G had reduced migratory ability. To evaluate the resistance to stress of the nuclear envelope, fibroblasts from subjects with HGPS and control individuals were heat shocked for 30 minutes at 45° C. Cells were then either fixed immediately (time 0) or incubated at 37° C. for 24 and 48 hours to allow for recovery (Paradisi et al., 2005). The control cells recovered rapidly from heat shock, since no changes were apparent in their growth rate 24 and 48 hours later. However, fibroblasts from the subject with HGPS were hypersensitive to heat shock, and recovery was only observed 48 hours after heat stress.

Characterization of the endogenous progerin in skin biopsy sections derived from a subject with HGPS: Understanding the cellular mechanism underlying the clinical sequelae of HGPS requires the identification of the cellular targets of the mutant LMNA G608G gene in vivo. Immunohistochemistry was performed on serial frozen sections from a skin biopsy derived from a 9 year-old subject with HGPS (HGADFN143). Lamin A/C was detected in the nuclei of most cells from the epidermis and in the dermal compartments. The anti-LMNA G608G antibody showed a very restricted pattern of distribution, with positively labeled nuclei localized within the blood vessel, some cells surrounding the sweat glands, and in cells of the arrector pili muscle. These results provide the first direct evidence that progerin is present in vascular cells in vivo and define a direct relationship between LMNA G608G mutation and atherosclerosis in HGPS.

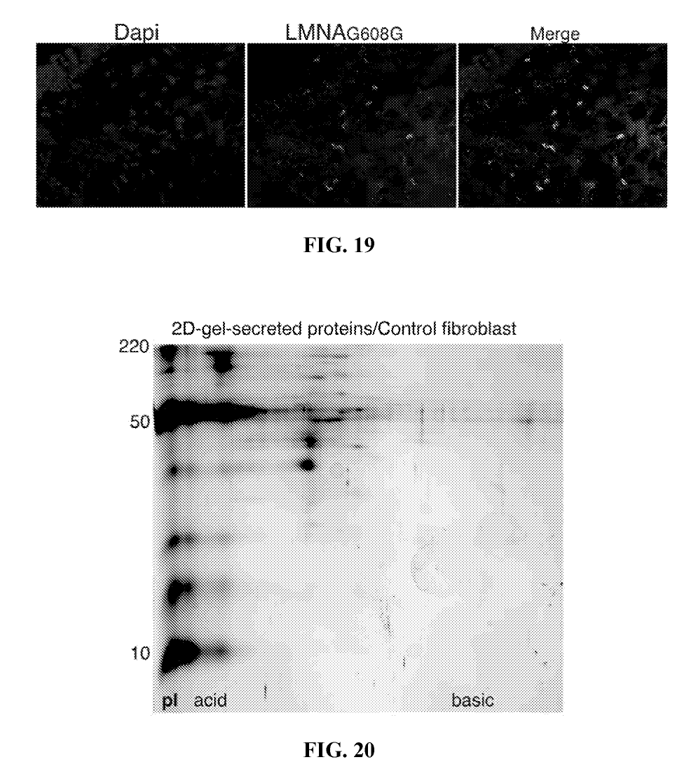

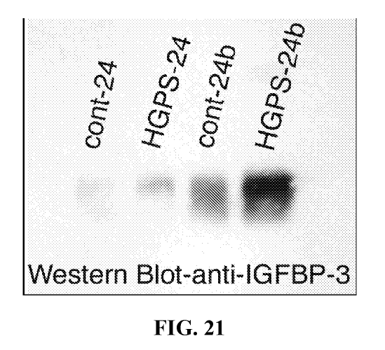

Lamin A G608G is present in skin derived from elderly individuals: Collection of skin samples (Table 4) was screened for the presence of progerin (mutant lamin A G608G) protein in situ on skin sections derived from elderly individuals. There was one positive sample out of the 20 samples tested. Immunohistochemical analysis was performed on 6 μm frozen skin sections fixed in methanol/acetone. The tissue specimen positively labeled derived from the forehead of an 85-year-old female. Only a few nuclei were positively labeled within the dermal compartment as shown in FIG. 19. These positively labeled nuclei were dermal fibroblasts and were not segregated in one area of the dermis, which could have indicated that these cells were derived from the same progenitor cell, but were distributed throughout the dermis. Further analyses will confirm the positive staining, and will verify the sequence of the mutant lamin A in this tissue. mRNA isolation from skin sections of this sample will allow for direct sequencing of the complementary lamin A cDNA after RT-PCR based assays. Using additional cellular markers, such as vimentin for dermal fibroblasts, will allow the identification of the cell type positively labeled with anti-progerin antibody in the tissue.

Additional skin samples from elderly individuals exhibiting a positive signal with anti-progerin and/or anti-prelamin A antibody can indicate a role for lamin A alteration in normal aging. HGPS is caused in nearly all-classic cases by a de novo mutation 1824C>T (also denoted G698G) in exon 11 of LMNA gene on chromosome 1 (De Sandre-Giovannoli et al., 2003; Eriksson et al., 2003). Moreover, patients with HGPS die in their teens from widespread atherosclerosis. It will be important to verify if the elderly individuals that develop such mutations as a function of age also develop atherosclerosis. Clinical information regarding the patients whose skin specimens were examined as described herein can be analyzed to determine whether subjects exhibit high blood pressure, atherosclerosis, or diabetes. The subject described supra has high blood cholesterol levels.

2-Dimensional gel analysis to study changes in secretomes derived from dermal fibroblast cultures: Analyses as described herein indicate that the mutant lamin A progerin appears to be expressed mostly in dermal fibroblasts from elderly subjects. This observation indicates that dermal fibroblasts are prone to DNA damage during aging. The secreted protein repertoire of dermal fibroblasts and its changes during the process of aging can be further analyzed. To define protein markers of aging, the protein complexity of secreted proteins from one adult control fibroblast culture have been analyzed. In one embodiment, proteins secreted in the culture medium of control cells were concentrated and separated by isoeletric focusing on a Protean IEF cell (Biorad) using 11 cm ready strips (pH gradient 3-10, Biorad). After a 12 hour rehydratation with the protein sample at room temperature, the strips were focused for a total of 35 kV h. The focused sample was submitted to electrophoresis on 12% w/v polyacrylamide gel and stained by silver stain method. FIG. 20 shows the 2-dimensional gel pattern from control fibroblasts. Several protein spots can be detected between a pI 3 to 7, and a molecular weight between 30 to 220 kD. Spots with low molecular weight are not well detected with these experimental conditions. A better resolution can be achieved by separating the protein sample on a 10% acrylamide gel. To increase the signal and the number of protein spots on 2D gel, the amount of protein loaded will be increased on the first dimensional separation step (IEF). Applying the fluorescent labeling steps to the protein sample prior separation will also increase the sensitivity of this technique and permit a differential in gel analysis between two samples within the same gel.

This analysis will allow designing and adjusting the experimental protocol to increase the complexity of protein spots that can be resolved by this method. Several aspects can be modified, including increasing the time of incubation to 48 hours in no serum before collection, as well as increasing the amount of total proteins to be analyzed.

Detection of Insulin growth factor binding protein (IGFBP-3) in culture medium derived from HGPS dermal fibroblasts: Previously, IGFBP-3 was found to accumulate in higher levels in medium conditioned by control fibroblasts at late population doublings (Goldstein et al., 1993). Condition medium was prepared from adult and HGPS dermal fibroblast cultures. Cells from early passage numbers were grown to 80% confluency and transferred to medium containing no serum for 24 hours. The medium was collected and concentrated by precipitation. Cells were allowed to recover for 48 hours in complete medium and then transferred for a second time to medium without serum for 24 hours. The second conditioned medium, denoted b, was collected. 30 μg of total proteins from the first and second conditioned medium preparations were probed with anti-IGFBP-3 antibody (Calbiochem) by Western blot analysis. The presence of IGFBP-3 was detected in culture medium derived from HGPS cells, already in the first preparation while in the control medium the signal was hardly detectable. Furthermore, HGPS cells submitted to medium without serum for a second cycle exhibited an increased amount of IGFBP-3 in their conditioned medium. Control cells treated in a similar manner showed only a small increased in IGFBP-3 signal. This result indicates that the secreted levels of IGFBP-3 differ between control adult and HGPS fibroblast cultures and that HGPS cells submitted to serum starvation twice exhibit an increased level of secreted IGFBP-3. Further analysis can determine whether fibroblast cultures derived from control elderly individuals exhibit a similar behavior as HGPS fibroblasts.

Signaling through the insulin/IGF axis is a major determinant of the rate of aging in many species (Barbieri et al., 2003). IGFBP-3 accumulates in conditioned medium of senescent human fibroblasts, indicating that it can contribute to the senescent phenotype. Defining how this factor can be altered in dermal fibroblasts from elderly subjects expressing aberrant lamin A can ink aging and premature aging processes. Moreover, the dissection of the repertoire of secreted proteins using a sensitive and reproducible method, such as 2-Dimensional in gel differential electrophoresis (DIGE), will allow identification of changes in the secreted proteins and their relation to normal and/or premature aging of the skin.

The mutant form of lamin A that causes Hutchinson-Gilford Progeria is a biomarker of cellular aging in human skin.

Lamins of the A- and B-type are intermediate filament proteins that constitute major components of the nuclear lamina, a filamentous meshwork forming an interface between the inner nuclear membrane and the chromatin (Aebi et al, 1986). Lamins A and C, the major isoforms of A-type lamins, are expressed in differentiated vertebrate cells (Hitchinson et al, 2004) and are translated from alternatively spliced transcripts of the LMNA gene. In contrast to the single LMNA gene, there are two B-type lamin genes: LMNB1 gene encodes lamin B1 protein (Lin et al, 1995; Wydner et al, 1996), while LMNB2 encodes two protein products by alternative splicing: lamin B2 and lamin B3, (Furukawa et al, 1993; Rober et al, 1989). The B-type lamins are expressed throughout development, and one or more B-type lamins are present in all cell types (Benevente et al, 1985; Stick et ak, 1985; Lehner et al, 1987)

Lamins are located at the nuclear lamina and throughout the nucleoplasm (Bridger t al, 1993; Hozak et al, 1995), where they seem to play fundamental roles in the shape, integrity and function of the nucleus and in DNA replication and RNA transcription (Shumaker et al, 2003). Lamin A and lamin B are modified at their carboxyl-terminal —CAAX box through a series of post-translational modifications. The modifications include, successively, farnesylation of the cysteine in the C-terminal CaaX motif (C, cysteine; a, aliphatic; X, any amino acid), followed by a proteolytic cleavage of the aaX-terminal tripeptide, and by methylation of the farnesylated cysteine (Sinensky et al, 1994). While B-type lamins remain permanently farnesylated, prelamin A (the precursor of mature lamin A) undergoes a second cleavage of the remaining 15 C-terminal residues (aa 647-661) to give rise to the mature lamin A, therefore losing its farnesyl modification (Sinensky et al, 1994; Young et al, 2004). The enzyme responsible for these sequential proteolytic cleavages is the zinc metalloproteinase ZMSPTE24, for which lamin A is the only known substrate in mammals (Corrigan et al, 2005).

Mutations in LMNA are implicated in 12 distinct disorders, commonly referred to as laminopathies, and involve different tissues, including muscle, peripheral nerve, adipose, bone and skin tissue. These disorders exhibit distinct clinical phenotypes associated with features such as myopathy, cardiomyopathy, lipodystrophy, neuropathy and premature aging (Rankin et al, 2006; Worman et al, 2007; Capell et al, 2006). The two best-known examples of accelerated aging syndrome in humans are Hutchinson-Gilford progeria syndrome (HGPS, ‘Progeria of childhood’) and Werner syndrome (WS, ‘Progeria of the adult’). Whereas most cases of WS have been caused by mutations in WRN helicase (Yu et al, 1996), a subset of WS patients do not show mutations at the WRN locus (atypical WS), but show heterozygous amino acid substitutions in the heptad repeat region of lamin A (Csoka et al, 2004; Plasilova et al, 2004; Cao et al, 2003).

Hutchinson Gilford progeria syndrome (HGPS, OMIM 176670) is a rare sporadic disorder with an incidence of 1 per 4-8 million live births, consisting of a premature aging phenotype with rapid growth deceleration in childhood (Capell et al, 2006). Appearance at birth and birth weight are can be normal, but growth is slowed by the age of one year (Brown et al, 1985). The phenotypic appearance consists of the following: short stature, sculpted nose, alopecia, prominent scalp veins, loss of subcutaneous fat, and dystrophic nails. In addition, HGPS patients show skeletal abnormalities that can reflect deficient osteogenesis, in the extremities, mandibular and cranial dysplasia with disorganized growth, deformations in dentition and severe osteolysis (Fernandez et al, 1992; Sweeney et al, 1992). Causes of death in HGPS subjects during the second decade of life are chronic conditions most common in elderly people, especially coronary artery disease and stroke due to widespread arteriosclerosis (Brown et al, 1992).

Nearly 90% of the subjects affected with HGPS carry a de novo G608G (GGC>GGT) mutation within exon 11 of LMNA, (Cao et al, 2003; De Sandre-Giovannoli; Eriksson et al, 2003). This single nucleotide change activates a cryptic splice donor site, which results in a deletion of the 3′ terminal 150 nucleotides of exon 11 of the mRNA, causing a 50 amino acid internal truncation near the carboxyl-terminus of prelamin A (Eriksson et al, 2003). The truncated lamin A, referred to as progerin, lacks amino acids 607 to 656 of prelamin A but retains the CAAX box (Cao et al, 2003; De Sandre-Giovannoli; Eriksson et al, 2003). Because the endoproteolytic cleavage site for ZMPSTE24 is lost in the mutant protein, progerin remains permanently farnesylated causing its tight association with the nuclear envelope and producing numerous nuclear envelope abnormalities (Goldman et al, 2004; McClintock et al, 2006). This modification also appears to affect the dynamic state of progerin within the lamina (Dhal et al, 2006; Delbarre et al, 2006).

Cells derived from HGPS individuals and subjects with pathologies resembling HGPS, such as atypical progeria Werner Syndrome (WS), Restrictive Dermopathy (RD), and Mandibular Dysplasia (MAD), appear to share a common feature: accumulation of prelamin A, either as full-length prelamin A protein or various truncated forms of prelamin A (Young et al, 2006). In these diseases, the prelamin A or mutant prelamin A remains farnesylated and accumulates within the nuclear compartment as cellular generation increases (Goldman et al, 2004; McClintock et al, 2006; Navarro et al, 2005). The persistence of the farnesylated form is the key element responsible for severe nuclear abnormalities and defects in heterochromatin organization, mitosis, DNA replication, transcription and repair (Shumaker et al, 2006; Toth et al, 2005; Capell et al, 2005; Liu et al, 2005).

Fibroblasts cultured from elderly individuals were found to exhibit nuclear phenotypes identical to those of HGPS cells (Scaffidi et al, 2006). While those cells expressed progerin mRNA transcripts at barely detectable levels (Scaffidi et al, 2006), long-term cultures contained a few abnormal nuclei that were clearly positive with anti-progerin specific antibody (Cao et al, 2007). These observations indicate that progerin is also expressed in normal cells. The cryptic splice donor site in exon 11 of LMNA is activated by the HGPS mutation, but the normal sequence is able to function in a similar fashion under some circumstances, at least in long-term culture.

In Vivo Detection of Progerin in Unaffected Individuals.

To address the biological relevance of progerin expression in unaffected individuals and its relationship to normal aging, the spatiotemporal expression pattern of progerin in human skin was examined at all ages. The results described herein show that progerin is expressed and accumulates in vivo during normal aging.

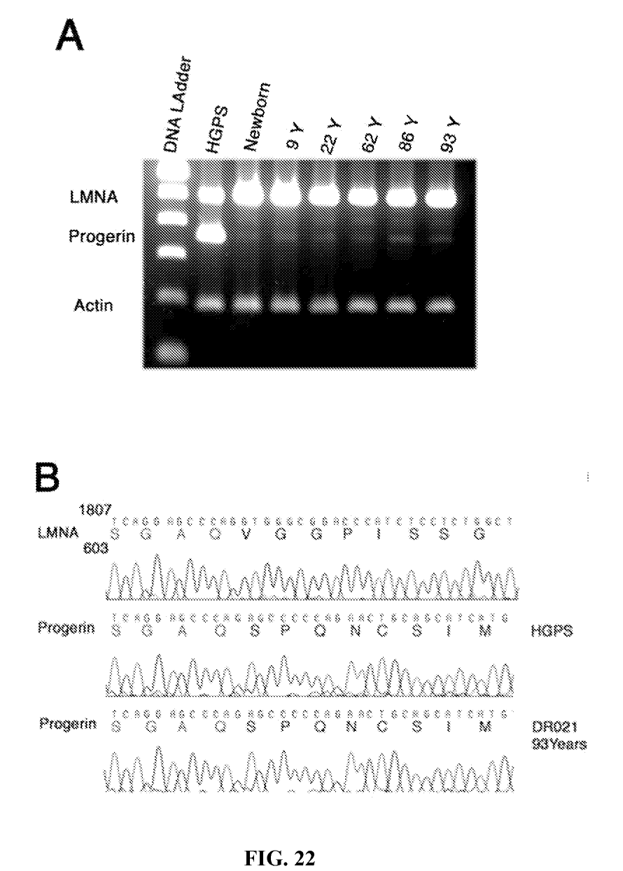

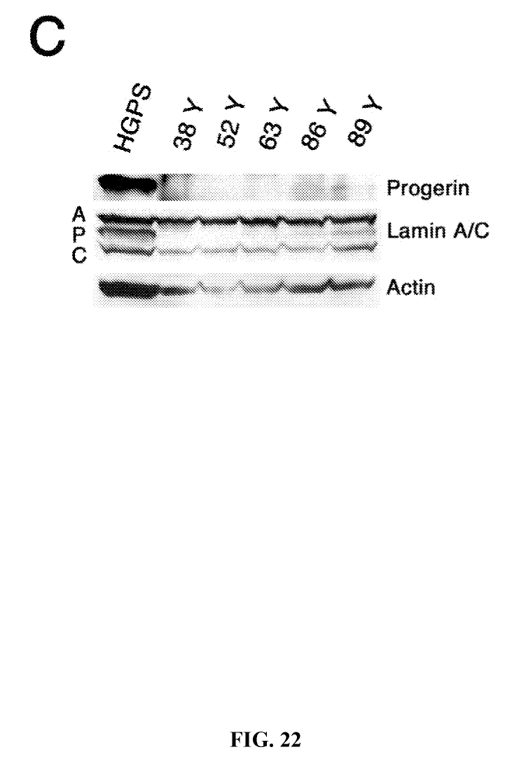

Human skin was used as a model system to investigate whether progerin is expressed in the skin of unaffected individuals. 150 skin biopsies from newborn foreskins and from unaffected individuals, including equal numbers of females and males ranging in age from 22 to 97 years were collected from the Dermatology Clinic at Columbia University. The biopsies originated from different body sites (Table 5). Using a one-step reverse transcription polymerase chain reaction (RT-PCR), total RNA preparations isolated from skin biopsies were screened. Primers in exon 9 and exon 12, described previously (Capell et al, 2005), primarily amplified wild type lamin A; however, a minor fragment similar in size to the HGPS transcript was detected in 50 biopsies, examples of which are shown in FIG. 22A. The levels of amplified short product remained low in the samples and no age-related differences were observed. Sequencing of the short cDNA product derived from RNA preparation of a 93 year-old donor was found to be identical to the progerin cDNA sequence (FIG. 22B), demonstrating that the progerin transcript, identified as an aberrant lamin A product in HGPS, can constitute a true physiological lamin A isoform. Another minor cDNA fragment migrating slightly higher than the progerin product was detected in the skin samples and was identified as the delta 10 isoform of lamin A (Machiels et al, 1996).

The ubiquitous presence of a low level of progerin mRNA in human skin also indicated that the protein, if present, can be expressed at very low levels. A rabbit polyclonal antibody specific to lamin A G608G (progerin) (McClintock et al, 2006) did not detect progerin in normal tissue, and to rule out that antibody binds with low affinity or that the recognized epitope can be masked, a rabbit monoclonal antibody was generated by determining the lamin A G608G amino acid sequence reading frame (De Sandre-Giovannoli et al, 2003; Paradisi et al, 2005). To generate a specific anti-Lamin A G608G antibody, a short peptide overlapping the region where the 50 amino acid internal deletion occurred in lamin A mutant G608G sequence was chosen, as described previously (McClintock et al, 2006). Three rabbits were immunized with the peptide using the standard protocol performed by Covance ImmunoTechnologies (Denver, Pa., USA). Preimmune and immune sera were characterized by Western blot analysis and indirect immunofluorescence on HGPS and control fibroblast cells. The serum of rabbit 972 specifically recognized progerin protein and gave no signal with A-type lamin or pre-lamin A. The spleen from Covance rabbit 972 was sent to Epitomics, Inc (Burlingame, Calif., USA). Lymphocytes were isolated from the spleen and fusion was performed according to Epitomics' standard protocol. Positive hybridomas were selected and the supernatants of the primary clones and subsequent subclones were screened by Western blot and indirect immunofluorescence analysis using control and HGPS dermal fibroblasts treated or untreated with FTI as described previously (McClintock et al, 2006). One clone, 972S9, was selected based on its specific reaction for progerin and was used in this study in addition to the polyclonal anti-progerin antibodies described herein.

Three rabbits were immunized. The serum of rabbit 972 specifically recognized progerin protein and gave no signal with A-type lamin including pre-lamin A. Spleen derived lymphocytes isolated from rabbit 972 were used to generate hybridomas (Epitomics, Inc.; Burlingame, Calif., USA). One clone, 972S9, was selected based on its specific reaction for progerin and was used in this study. 40 skin biopsies of different ages for progerin expression were screened by Western blot analysis, and as indicated in a representative blot, a small amount of progerin protein was present in skin samples derived from elderly individuals (FIG. 22C). Progerin was not detected in protein extracts derived from young adult skin under the experimental conditions, but low levels of expression were clearly detected in samples derived from old skin and appeared to increase slightly with the increasing chronological age of the donor (FIG. 22C).

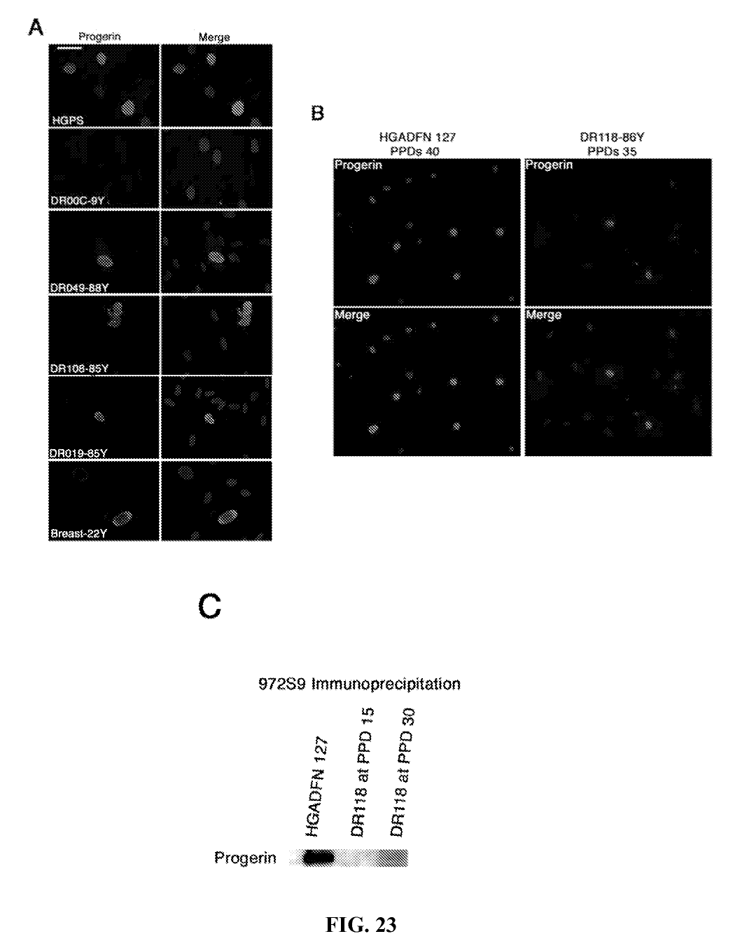

Normal fibroblasts overexpressing progerin recapitulate HGPS cellular phenotype. Primary fibroblast cultures from skin biopsies of normal individuals at different ages were established (Table 5). Primary fibroblast cultures were made using two different methods by explant outgrowth or by enzymatic dissociation of the dermis (Wang et al, 2004). Indirect immunofluorescence analyses were performed with the anti-progerin monoclonal antibody on fibroblast cultures at early population doublings (PPDs). HGPS cultures below 25 PPDs showed that 27% of nuclei were progerin-positive. In primary fibroblast cultures from unaffected individuals, a few nuclei showed a positive staining (FIG. 23A). Independently of the method used for their establishment, cultures derived from young subjects (22 to 30 years) showed less than 0.01% progerin-positive staining, while cultures derived from elderly subjects exhibited an average of 0.3% to 0.8% progerin-positive nuclei at early PPDs. Progerin-positive cells from normal individuals showed nuclear abnormalities similar to those observed in HGPS fibroblasts (FIG. 23A). Nuclear blebs, nuclear envelope invaginations, binucleated cells and large nuclei, reminiscent of abnormal cell cycle exit, were observed in cultures derived from elderly individuals, as was recently reported (Cao et al, 2007).

TABLE 5
Primary fibroblast cultures from skin biopsies
of normal individuals at different ages.

| Age | Body Site | Number of Subjects |
|---|---|---|
| Newborn | Foreskin | 8 M |
| 22 to 46 | Breast | 6 F |
| 30 to 55 | Face (Cheek, Forehead, Scalp, Ear, | 8 F/10 M |
| | Nose) Leg | |
| 56 to 70 | Face (Temple, Cheek, Forehead, | 17 F/10 M |
| | Scalp, Ear, Nose) | |
| 56 to 70 | Neck, Chest, Leg, Back, Hand | 5 F/6 M |
| 71 to 97 | Face (Temple, Cheek, Forhead, | 26 F/33 M |
| | Scalp, Ear, Nose) | |
| 71 to 97 | Neck, Chest, Arm, Leg, Back, | 10 F/11 M |
| | Hand | |

When fibroblast cultures were serially passaged to approach the end of their lifespan, HGADFN 127 cells (HGPS) ceased to grow when they reached approximately 40 to 45 PPDs under the culture conditions. Indirect immunofluorescence analyses with anti-progerin rabbit mAb 972S9 of HGPS cultures showed an average of 27% of progerin-positive nuclei at early PPDs, while towards late PPDs (above 35) nearly 90% of the cells were progerin-positive (FIG. 23B). When similarly studies were performed with normal fibroblast cultures, the number of cells positively labelled with anti-progerin Ab increased slightly with the cellular-age in vitro. As exemplified, fibroblast culture DR118 established from an 86 year-old female was monitored by indirect immunofluorescence with anti-progerin mAb and exhibited an average of 0.4% of progerin-positive cells in young cultures. That average increased to 0.8% in late cultures (PPDs 30 to 35). Western blot analysis of total protein extracts isolated from primary fibroblast cultures derived from adult and elderly individuals exhibited no progerin signal when probed with anti-progerin mAb, while progerin was readily detected in total HGPS cellular extracts at early PPDs. Progerin accumulates in HGPS fibroblasts in a cellular-age-dependent manner (McClintock et al, 2006). To verify further whether normal fibroblasts can accumulate progerin in a similar fashion to the HGPS counterpart, immunoprecipitation assays were performed with anti-progerin mAb using nuclear extract preparation. Immunoprecipitation of progerin was performed on isolated nuclei preparation from an average of 6×10⁶ of normal cells from elderly individuals at early and late PPDs (FIG. 23C). Western blot analyses of immunoprecipitated materials were probed again with anti-progerin mAb. As indicated in FIG. 23C, a low amount of progerin can be detected at late PPDs in a representative fibroblast culture, DR118 that was established from an 86 year-old female (FIG. 23C).

Collectively, these results indicate that low levels of progerin protein are present in skin derived from elderly individuals as well as in primary dermal fibroblast cultures established from this population of subjects. Importantly, progerin appeared to accumulate in normal fibroblast cultures in a cellular-age-dependent manner, but remained still relatively low when compared to the amount of progerin accumulating in HGPS cultures (Goldman et al, 2004; McClintock et al, 2006).

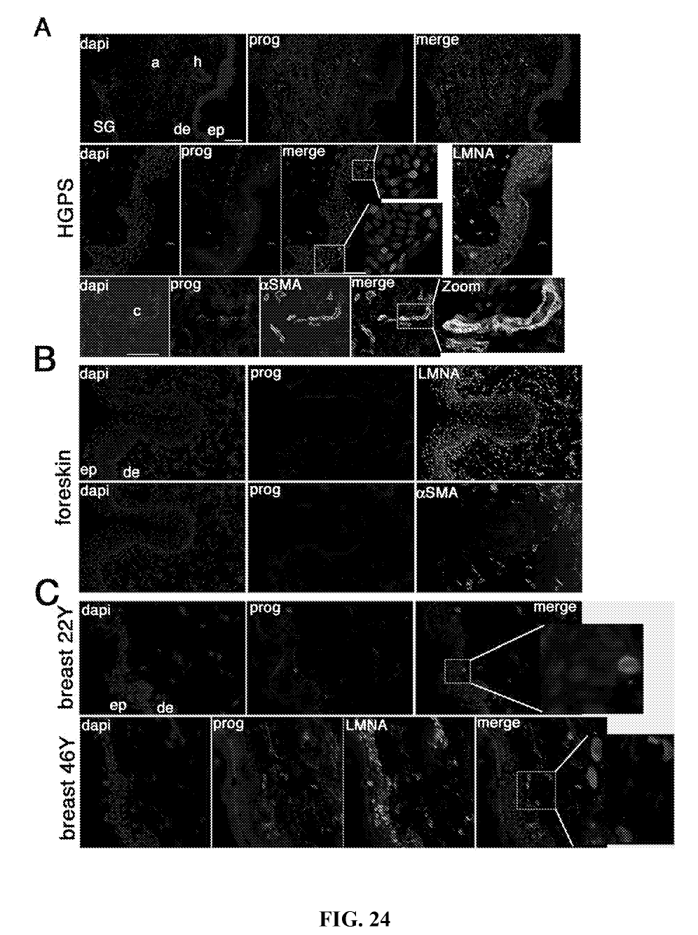

In vivo profiling of progerin protein on skin biopsies derived from HGPS and unaffected individuals using an anti-progerin monoclonal antibody. To gain insight into the functional impact of the progerin isoform, human skin biopsies were examined to determine the cellular distribution of progerin in vivo. In skin sections derived from a 9 year-old subject with HGPS (HGADFN143), progerin was localized in vascular cell nuclei and in nuclei throughout the dermis (McClintock et al, 2006). Using the monoclonal anti-progerin Ab, sections from the same HGPS sample were reanalyzed. Progerin was detected within dermal nuclei, blood vessels, arrector pili muscle, and cells surrounding the sweat glands (FIG. 24A). Furthermore, progerin was detected in keratinocyte nuclei in the upper most layers of the epidermis and localized into a thick rim like structure at the nuclear envelope. This distribution indicates that a subset of terminally differentiated keratinocytes accumulates progerin in HGPS (FIG. 24A).

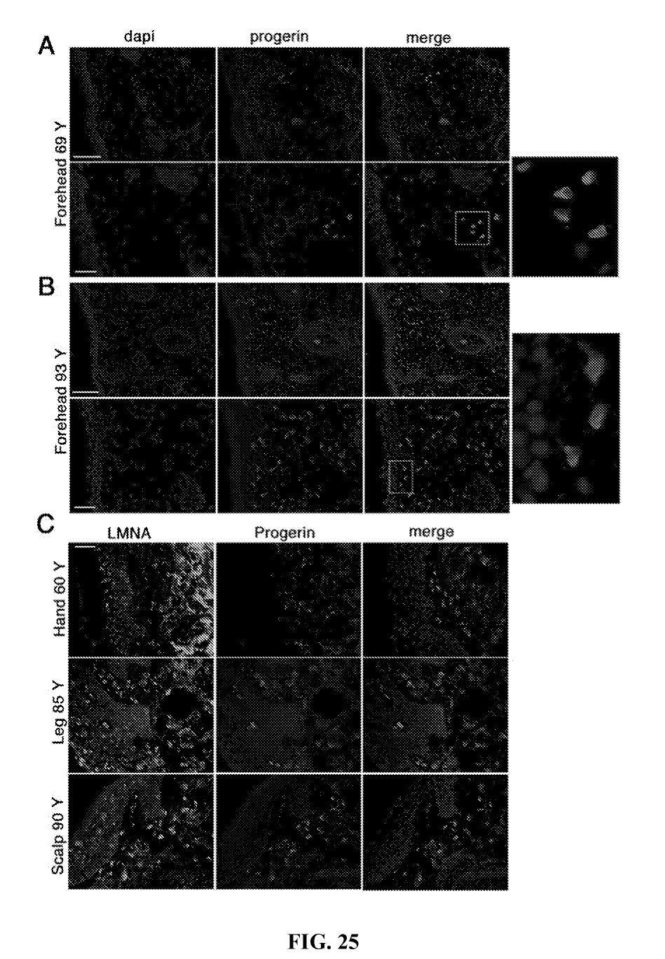

Low levels of progerin mRNA were detected in total skin mRNA preparations at all ages (FIG. 23A) and low amounts of protein were detected in elderly skin extracts (FIG. 23C). To determine whether progerin is present at low levels or in only a few cells, immunohistochemistry was performed on sections derived from newborn foreskins and sixty skin biopsies from different body sites (Table 5) of 22 to 93-year-old subjects. Representative patterns of progerin localization are shown in FIGS. 24 and 25. Newborn foreskin exhibited no signal with anti-progerin mAb, while the nuclei showed a positive signal with anti-lamin A Ab (FIG. 24C, panel LMNA). The upper dermis on serial foreskin sections was highly vascularized; numerous vascular loops were labelled with anti-α smooth muscle actin Ab (αSMA) (FIG. 24B). Screening of five foreskin biopsies detected no progerin in any skin compartment. Breast skin sections from a 22-year-old female showed a few progerin-positive nuclei close to the basement membrane and in the papillary dermis (FIG. 24C). Breast skin sections from a 46-year-old woman exhibited a greater number of progerin-positive nuclei dispersed throughout the papillary dermis (FIG. 24C).

Sections from the forehead of a 69-year-old male, showed progerin-positive nuclei mostly in the upper dermis (FIG. 25A). Skin sections from the forehead of a 93-year-old female exhibited a high density of progerin-positive nuclei in the upper and lower dermis (FIG. 25B). A similar pattern of progerin distribution was detected in skin sections from different body sites (FIG. 25C) including the hand (60 years), leg (85 years) and scalp (90 years). Progerin was detected as strong nuclear rim staining in fibroblasts located in the upper, middle and deep dermal compartments in elderly individuals (FIGS. 25B, and 25C), while in young individuals, a few fibroblasts accumulating progerin were localized in the papillary dermis close to the basement membrane. With increasing age, the number of positive cells increased in the upper dermis and progressively extended more deeply, creating a gradient of progerin-positive fibroblasts from the basement membrane to the reticular dermis. Skin derived from both sun-exposed areas and non-exposed areas shared a similar distribution of progerin-positive fibroblasts (FIGS. 24 and 25).

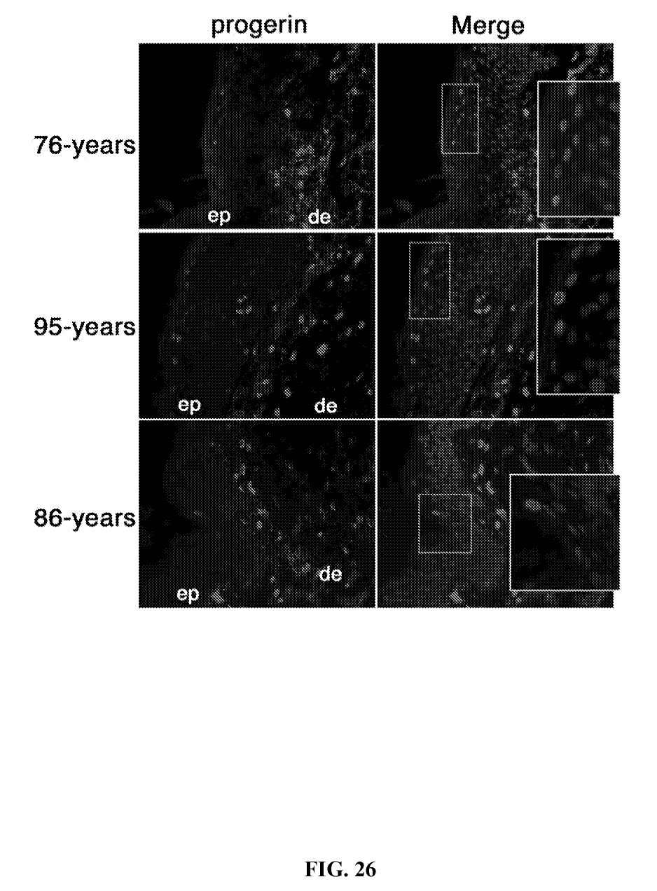

Progerin was detected in a subset of terminally differentiated keratinocytes in some skin biopsies derived from elderly individuals. In the vast majority of unaffected skin biopsies tested herein, progerin was not detected in the epidermis, as shown in FIG. 24 to 25. However, in some skin biopsies derived from 70 to 95 year-old subjects, a positive progerin signal was detected within the epidermis. Representative examples of skin sections exhibiting a progerin-positive signal in the epidermis are shown in FIG. 26. When progerin was detected in elderly epidermis, only a few keratinocytes per section were positively labelled with anti-progerin mAb in the uppermost layers of the epidermis and the signal was never as bright as the one harbored by dermal fibroblasts (FIG. 26). Skin sections from 76- and 95-year-old females showed the highest number of positively labelled keratinocytes in the granular layer of the epidermis (FIG. 26). However, the progerin-positive keratinocytes were not uniformly distributed throughout the entire length of the epidermis on the sections but rather were localized within a small area. In most elderly skin sections, only one to three keratinocytes were sporadically observed in the upper most layer of the epidermis, as shown on an 86 year-old male skin section (FIG. 26). In these terminally differentiated cells, progerin signal was localized into a rim-like structure at the nuclear periphery (FIG. 26). Overall, the progerin signal was weak compared to the strong signal obtained in dermal fibroblasts.

These results indicate that progerin accumulates in terminally differentiated keratinocytes that are localized within the layers closer to the skin surface. Moreover, the progerin-positive keratinocytes exhibited a thick nuclear rim staining, again reminiscent of staining observed in progerin-positive keratinocytes from HGPS skin sections (FIG. 24A). Since only very few keratinocytes from the upper epidermis were labelled with anti-progerin antibody, the possibility of epitope masking, which can occur in lamin detection, can not be ruled out. Different fixation methods were used in combination with different permeabilization procedures but the staining remained unchanged. The distribution and localization of the few progerin-positive keratinocytes were consistent with observations of skin sections derived from the patient with HGPS (FIG. 24A). As in HGPS keratinocytes, the normal keratinocytes were terminally differentiated as defined by their location in the uppermost layer of the granular layer (Fuchs et al, 1995). In addition, those progerin positive-keratinocytes exhibited identical rim-like staining at the nuclear envelope periphery. While a progerin-positive signal was sporadically detected in keratinocytes from the interfollicular dermis, no signal was detected in the different epidermal appendages and vasculature system regardless of body site or age of the donor sample under the experimental conditions.

These data show that sporadic use of the cryptic splice site in exon 11 of LMNA occurs in vivo in normal cells, however that site is much more active in HGPS cells carrying the LMNA G608G mutation. Progerin mRNA transcripts were detected at very low levels in skin samples of unaffected individuals and remained fairly constant at all ages (FIG. 22). A similar observation was previously reported using fibroblast cultures derived from healthy individuals (Scaffidi et al, 2006).

While the progerin mRNA can be detected at uniform levels by RT-PCR analysis in tissue samples, the protein apparently accumulated with age. Western blot analysis was capable of detecting low levels of progerin in skin samples from elderly individuals (FIG. 22C), but the protein was below limit of detection in young samples. In skin biopsies from young individuals, progerin was either absent or present only at low levels, since it was not detectable by indirect immunofluorescence (FIG. 24). With advancing age, concomitant with alterations of the dermal compartment (such as disorganized connective tissue), progerin accumulated in skin cells but was restricted to specific cell types, primarily dermal fibroblasts and terminally differentiated keratinocytes. Thus, progerin-positive cells were not randomly distributed within the skin (FIG. 24 to 26). In the dermis, progerin-positive fibroblasts appeared first at the basement membrane and their number increased and spread throughout the dermis with age (FIGS. 24 and 25).

Dermal fibroblasts are the major cell type in the dermis and represent a heterogeneous population of cells based on differences in proliferation potential and extracellular matrix protein synthesis (Sorrell et al, 2004). The results described herein show that the population of progerin-positive fibroblasts can be a terminal differentiation phenotype of dermal fibroblasts in vivo. The results show that progerin-positive cells are not randomly distributed in the dermis, but appear in an age-related fashion at the basement membrane and gradually spread throughout the dermal compartment (FIGS. 24 and 25). While a high number of progerin-positive cells were detected in skin biopsies of older individuals, only a few of them were found in the respectively derived fibroblast cultures (FIG. 23), indicating that the progerin-positive cells have reached terminal growth arrest, and were not able to propagate in culture. However, their number increased slightly in late passage cultures indicating that progerin accumulates in cells that have entered a stage of terminal differentiation or senescence.

Progerin build-up in HGPS cells occurs in a cellular-age-dependent manner (Goldman et al, 2004; McClintick et al, 2006). Moreover, accumulation of progerin has a negative impact on cell cycle progression and elicits reduced migration in HGPS fibroblasts (McClintick et al, 2006; Cao et al, 2007). Altogether, these findings indicate that progerin-positive fibroblasts in vivo define a subpopulation of fibroblasts that have reached terminal differentiation and/or senescence.

The epidermis is a self-renewing stratified epithelium mainly composted of keratinocytes that undergo a complex and dynamic program of terminal differentiation throughout life (Sorrell et al, 2004). This process starts when proliferating keratinocytes of the basal layer move upward to the suprabasal layers and progressively acquire the ability to express sequentially the specific gene products that are required for differentiation (Volz et al, 2003). Terminally differentiated keratinocytes of the granular layer are in a transitional stage on their way to forming the cornified layer of the epidermis (Sorrell et al, 2004). This transition is accompanied by the elimination of organelles including the nuclei and changes in cytoplasmic functions. These cellular events allow the production of the outermost epidermal layer, composed of the flattened, enucleated, dead cornified cells (corneocytes) that ensure the skin barrier function (Sorrell et al, 2004). It is within this transitional epithelial layer that progerin appears within a small subset of keratinocytes. These cells not only have reached terminal differentiation but also are about to extrude their nuclei and as such can be considered as having reached the end of their lifespan. Such terminal differentiation and transitional stages between nucleated and enucleated cells must indicate not only an important rearrangement of the nucleus but must also indicate a large-scale genome reorganization designed to switch off gene expression that is no longer required. Such remodelling of the nuclear lamina composition and changes in heterochromatin epigenetic marks to silence gene expression and overall chromatin organization must synergistically and actively participate in the settings of nuclear exclusion. During this final stage of terminal differentiation, the keratinocytes must have reached the end of their lifespan in vivo and can be regarded as senescent cells (Campisi et al, 2001; Campisi et al, 2005).

Replicative senescence is a permanent state of proliferation arrest (Funanyama et al, 2007). Terminal differentiation defines cells that permanently exit the cell cycle in the course of acquiring functional specialization. Both cellular stages share some common properties including stable cell-cycle arrest, flattened and enlarged morphology, increased cytoplasmic enzymatic vesicles, and changes in chromatin condensation and in gene expression patterns (Francastel et al, 2000; Narita et al, 2003).

Progerin is linked to the pathogenesis of HGPS and exerts a toxic nuclear effect because it remains permanently farnesylated (Capell et al, 2006). Cells derived from subjects with HGPS exhibit dysmorphic nuclei with significant changes in nuclear shape, including nuclear envelope invaginations, thickening of the nuclear lamina, loss of peripheral heterochromatin, and clustering of the nuclear pores (Goldman et al, 2004; McClintock et al, 2006). The changes in the lamina protein composition appear to be responsible for the disruption and loss of peripheral chromatin in HGPS cells, and has been linked to impaired epigenetic histone markers and genomic instability (Shumaker et al, 2006).

The results disclosed herein further show the dynamic changes in the nuclear lamina composition with the incorporation of an “age-associated” lamin isoform progerin during the process of keratinocyte differentiation in vivo. Analogous to the process that occurs in HGPS cells, the build-up of progerin in the nuclear lamina in normal cells contributes to the delocalisation of heterochromatin clusters away from the nuclear periphery, thereby contributing to the large scale-genome decondensation to allow genomic reorganization. This reorganization is requisite to ensure terminal differentiation and/or senescence. The association between progerin build-up to terminal differentiation and/or senescence in normal cells indicates that in the context of HGPS cells, progerin accumulation can trigger abnormal differentiation and early senescence which can prematurely deplete the pool of mitotic cells with renewal potency from tissue and cause the clinical sequelae that characterize old age.

As progerin is a cellular aging biomarker for dermal fibroblasts and keratinocytes of normal individuals in vivo, HGPS is a model for learning more about normal aging. Therapeutic strategies already tested on HGPS cells, such as farnesyltransferase inhibitors, can be useful in preventing normal aging and slowing progression of other age-related pathologies.

Materials and Methods

Production and Characterization of a Rabbit Monoclonal Anti-Lamin a G608G Antibody.

The lamin A G608G amino acid sequence reading frame was determined previously (De Sandre-Giovannoli et al, 2003; Paradisi et al, 2005). To generate a specific anti-Lamin A G608G antibody, a short peptide overlapping the region where the 50 amino acid internal deletion occurred in lamin A mutant G608G sequence as chosen, as described previously (McClintock et al, 2006). Three rabbits were immunized with the peptide using the standard protocol performed by Covance ImmunoTechnologies (Denver, Pa., USA). Preimmune and immune sera were characterized by Western blot analysis and indirect immunofluorescence on HGPS and control fibroblast cells. The serum of rabbit 972 specifically recognized progerin protein and gave no signal with A-type lamin or pre-lamin A. The spleen from Covance rabbit 972 was sent to Epitomics, Inc (Burlingame, Calif., USA). Lymphocytes were isolated from the spleen and fusion was performed according to Epitomics' standard protocol. Positive hybridomas were selected and the supernatants of the primary clones and subsequent subclones were screened by Western blot and indirect immunofluorescence analysis using control and HGPS dermal fibroblasts treated or untreated with FTI as described previously (McClintock et al, 2006). One clone, 972S9, was selected based on its specific reaction for progerin and was used in this study.

Human Skin Biopsy Sections and Primary Dermal Fibroblast Cultures

Normal skin biopsies and newborn foreskins were obtained from the Dermatology Clinic in accordance with the health research ethics board of Columbia University. The biopsies originated from different body sites and were obtained from equal numbers of males and females. The sex, age and body site of each donor was recorded (Table 1). Skin biopsies were embedded in Optimum Cooling Temperature medium (O. C. T.) and cryopreserved for tissue sectioning. Serial 6 μm frozen skin sections were prepared and stored at −80° C. In addition, small pieces were snap frozen in liquid nitrogen at the time of their collection for mRNA and protein extractions. Primary cultures of dermal fibroblasts were established using explant culture or enzymatic digestion of the skin as described previously (Wang et al, 2004).

Briefly, skin tissue was rinsed in PBS and incubated overnight in a Dispase II solution at 4° C. The epidermis and dermis were mechanically separated and primary HDF cultures were established using explants or enzymatic digestion methods of the dermis. For explant culture, the dermal portion of the skin was washed in PBS supplemented with penicillin and streptomycin cut into small pieces, and spread onto a culture dish. Culture medium, DMEM supplemented with 15% fetal bovine serum, was added. Fibroblast outgrowth started at day 3 to 7; the skin pieces were removed after a week and cultures were grown to 70% confluence. For HDF cultures established by enzymatic dissociation of the skin, dermal pieces were transferred to a solution of Collagenase I (Worthington Biochemical) at 200 units/mL of 1×PBS including 0.3 mM CaCl₂. The pieces were kept at 37° C. shaking for 1 to 4 hours until they were digested. The solution with the dermal fragments was then diluted 5-fold with complete DMEM medium (15% FBS, 1% penicillin-streptomycin, 1% L-glutamine), passed over a 70 μm cell strainer (BD Falcon) and centrifuged. The cell pellet was resuspended in complete medium and the cells were plated, grown to 70-80% confluence and cryopreserved at −80° C.

HGPS Skin Biopsy and Dermal Fibroblast Cells.

Dermal fibroblasts derived from HGPS patients carrying the LMNA mutation G608G (HGADFN001, HGADFN003, and HGADFN127) were grown as described previously (McClintock et al, 2006). The Progeria Research Foundation kindly provided the HGPS cells and frozen skin sections derived from a skin biopsy of a 9 year-old donor with HGPS carrying LMNA G608G mutation (HGADFN143).

One Step-Reverse Transcription/Polymerase Chain Reaction (RT-PCR)

Total RNA was extracted from skin biopsy pieces using an RNA extraction kit as per the manufacturer (QIAGEN). 11 g of RNA from each sample was submitted to one step RT-PCR (OneStep RT-PCR, QIAGEN) using primers located in exon 9 and 12 of lamin A (forward primer: 5′ GGCTGCGGGAACAGC 3′ (SEQ ID NO: 55), and reverse primer: 5′ CTGGCAGGTCCC 3′) (SEQ ID NO: 56) described previously (Scaffidi et al, 2006), together with primers located in human β actin isoform (Forward: CCCAGCACAATGAAGATCAA (SEQ ID NO: 57) and reverse GTGTAACGCAACTAAGTCAT (SEQ ID NO: 58)) as internal control. 50 μl reactions were submitted to reverse transcriptase for 30 minutes at 50° C., followed by PCR activation step at 95° C. for 15 minutes. PCR conditions were 35 cycles, each cycle consisting of 30-sec denaturation at 94° C., 30-sec annealing at 55° C., and 30-sec polymerization at 72° C. 15 μl of the reaction was analysed on 2% agarose gel and stained with ethidium bromide.

Indirect Immunofluorescence

Primary cultures of dermal fibroblasts from patients and controls were processed for indirect immunofluorescence as described previously (Paradisi et al, 2005). Mouse anti-lamin A Jo14 (Serotec) or clone 133A2 (abcam), anti-lamin A/C 131C3 (abcam), and anti-human a smooth muscle actin, clone 1A4 (DakoCytomation) were purchased. The secondary antibodies were affinity purified Alexa Fluor 488 goat or donkey IgG antibodies (Molecular Probes) and Cy3-conjugated IgG antibodies (Jackson ImmunoResearch laboratories). All samples were also counterstained with DAPI (Sigma-Aldrich).

Immunohistochemistry was performed on 6 μm frozen sections fixed by methanol/acetone (IV/IV) at −20° C. for 10 minutes and washed in PBS, then blocked in PBS buffer containing 3% BSA, 10% normal goat serum and 0.3% Triton X-100 for 30 minutes and 1 hour in the same buffer without Triton X-100. Slides were incubated with the monoclonal anti-progerin antibody for 1 hour. After 6 washes in blocking buffer, slides were incubated with donkey anti-rabbit affinity purified Cy3-conjugated IgG antibodies. Slides were washed in blocking buffer and in PBS, then mounted with Vectashield mounting medium (Vector Inc.)

Western Blot Analysis

Skin tissues were extracted in cytoskeleton buffer (CSK) (100 mM NaCl, 300 mM sucrose, 10 mM PIPES (pH 6.8), 3 mM MgCl₂, 0.5% triton X-100) and protease inhibitors (Roche) for 15 minutes on ice. Skin pieces were minced and homogenized with PowerGen 125 (Fisher Scientific) in CSK buffer. The insoluble material was digested in PBS buffer containing 100 μg/ml of DNAse 1 and 100 μg/ml of RNAse A (Sigma) for 20 minutes at 20° C.; after centrifugation the remaining pellet was resuspended in Laemmli sample buffer (BioRad) and boiled 5 minutes at 95° C. Equal amounts of extracts were loaded in parallel on a 7.5% polyacrylamide gel. After separation by electrophoresis, proteins were transferred to nitrocellulose membranes and incubated with blocking buffer as described previously (McClintock et al, 2006). Membranes were incubated with primary antibodies (Ab 972S9, anti-lamin A/C kindly provided by Dr. N. Chaudhary, anti-actin Ab (Sigma)), washed, and then incubated with the corresponding secondary antibody coupled to horseradish peroxidase (Jackson ImmunoResearch Laboratories). Proteins were visualized using the enhanced chemiluminescence detection system (Amersham Pharmacia Biotech).

Western blot analysis of progerin expression in normal fibroblast cultures established from an 86-year-old female was performed after immunoprecipitation of nuclei extract prepared from early (PPD 15) and late population doublings (between 30 to 35 PPDs). Nuclei were isolated from normal cell pellets, containing an average of 6×10⁶ cells, and from HGPS cell pellets, containing 2×10⁶ cells at early PPDs (below 25), in buffer A (10 mM HEPES (pH7.9), 1.5 mM MgCl₂, 10 mM KCL) supplemented with protease inhibitor cocktail tablet (Roche Applied Science). After 10 minutes incubation on ice, the preparation was dounce homogenized by 25 strokes followed by 10 minutes centrifugation at 800 g at 4° C. Nuclei pellets were resuspended in RIPA buffer supplemented with protease inhibitors and were briefly sonicated on ice and centrifuged at 4° C. Extracts were incubated with 10 μg of purified 972S9 IgG (anti-progerin rabbit monoclonal) for 4 hours at 4° C.; 30 μl of protein A sepharose, equilibrated in RIPA buffer, was added and the extracts were incubated for another hour at 4° C. After four washes in RIPA buffer and two washes in PBS, the IP pellets were resuspended in 50 μl Laemmli sample buffer (BioRad), separated on SDS-PAGE gels, and transferred onto nitrocellulose. Westerns were probed with anti-progerin rabbit mAb 972S9 and further processed as described above.

### REFERENCES

Adjei, A. A., Davis, J. N., Erlichman, C., Svingen, P. A. & Kaufmann, S. H. (2000) Clinical Cancer Research 6, 2318-2325.
Aebi, U., Cohn, J., Buhle, L. & Gerace, L. (1986) Nature (London) 323, 560-564.
Allsopp, R. C., Vaziri, H., Patterson, C., Goldstein, S., Younglay, E. V., Futcher, A. B., Gireider, C. W. and Harley, C. B. (1996). Telomere length predicts replicative capacity of human fibroblasts. Proceedings of the National Academy of Sciences 89, 10114-10118.
Barton, R. M. and Worman, H. J. (1999). Prenilated prelamin A interacts with Narf, a novel nuclear protein. Journal of Biological Chemistry 274, 30008-30018.
Benevente R, Krohne G, Franke W (1985) Cell type specific expression of nuclear lamina proteins during developement of xenopus laevis. Cell 41: 177-190.
Bergo, M. O., Gavino, B., Ross, J., Schmidt, W. K., Hong, C., Kendall, L. V., Mohr, A., Meta, M., Genant, H., Jiang, Y. et al. (2002). Zmpste24 deficiency in mice causes spontaneous bone fractures, muscle weakness, and a prelamin A processing defect. Proceedings of the National Academy of Sciences 99, 13049-13054.
Bione, S., Maestrini, E., Rivella, S., Mancini, M., Regis, S., Romeo, G. & Toniolo, D. (1994) Nature Genetics 8, 323-327.
Bridger J M, Kill I R, O'Farrell M, Hutchinson C J (1993) Internal lamin structures within GI nuclei of human dermal fibroblasts. Journal of Cell Science 104: 297-306.
Bridger, J. M. & Kill, I. R. (2004) Experimental Gerontology 39, 717-724. Brown W T (1992) Progeria: a human disease model of accelerated aging. American Journal of Clinical Nutrition 55: 1222S-12124S.
Brown W T, Kieras F J, Houck G E, Dutkowski R, Jenkins E C (1985) A comparison of adult and childhood progerias: Werner syndrome and Hutchinson Gilford syndrome. Advance Experimental Medical Biology 190: 229-244.
Brown, W. T., Kieras, F. J., Houck, G. E., Dutkovski, R. and Jenkins, A. (1992). A comparison of adult and childhood progerias: Werner syndrome and Hutchinson Gilford syndrome. Advance Experimental Medical Biology 190, 229-244.
Burke, B. and Stewart, C. L. (2002). Life at the edge: the nuclear envelope and human disease. Nature Reviews Molecular cell Biology 3, 575-585.
Campisi J (2001) Cellular senescence as a tumor-suppressor mechanism. Trends in Cell Biology 11: 27-31.
Campisi J (2005) Senescent cells, Tumor Suppression, and Organismal Aging: Good Citizens, BAd Neighbors. Cell 120: 513-522.
Cance, W. G., Chaudhary, N., Worman, H. J., Blobel, G. & Cordon-Cardo, C. (1992) Journal of Experimental Clinical and Cancer Research 11, 233-246.
Cao H, Hegele R A (2003) LMNA is mutated in Hutchinson-Gilford progeria (MIM 176670) but not in Wiedemann-Rautenstrauch progeroid syndrome (MIM 264090). Journal of Human Genetics 48: 271-274.
Cao K, Capell B, Erdos M R, Djabali K, Collins F S (2007) A Lamin A protein isoform overexpressed in Hutchinson-Gilford progeria syndrome interferes with mitosis in progeria and normal cells. Proceedings of the National Academy of Sciences 104: 4949-4954.
Capell B C, Collins F S (2006) Human laminopathies: nuclei gone genetically awry. Nature Review Genetics 7: 940-952.
Capell, B. C., Erdos, M. R., Madigan, J. P., Fiordalisi, J. J., Varga, R., Conneely, K. N., Gordon, L. B., Der, C. J., Cox, A. D. & Collins, F. S. (2005) Proceedings of the National Academy of Sciences 102, 12879-12884.
Chaudhary, N. & Courvalin, J. C. (1993) Journal of Cell Biology 122, 295-306.
Coaxum, S., Martin, J. L. and Mestril, R. (2003). Overexpression of heat shock proteins differentially modulates protein kinase C expression in rat neonatal cardiomyocytes. Cell Stress Chaperones 8, 297-302.
Corrigan D P, Kuszczak D, Rusinol A E, Thewke D P, Hrycyna C A, et al. (2005) Prelamin A endoproteolytic processing in vitro by recombinant Zmspte24. Biochemical Journal: 129-138.
Corrigan, D. P., Kuszczak, D., Rusinol, A. E., Thewke, D. P., Hrycyna, C. A., Michaelis, S, and Sinensky, M. S. (2004). Prelamin A endoproteolytic processing in vitro by recombinant Zmspte24. Biochemical Journal.
Csoka, A. B., Cao, H., Sammak, J. P., Constantinescu, D., Schatten, G. P. and Hegele, R. A. (2004). Novel lamin A/C gene (LMNA) mutations in atypical progeroid syndromes. Journal of Medical Genetic 41, 304-308.
Dahl K L, Scaffidi P, Islam M F, Yodh A G, Wilson K L, et al. (2006) Distinct strutural and mechanical properties of the nuclear lamina in Hutchinson-Gilford progeria syndrome. Proceedings of the National Academy of Sciences 103: 10271-10276.
De Sandre-Giovannoli, A., Bernard, R., Cau, P., Navarro, C., Amiel, J., Boccaccio, I., Lyonnet, S., Stewart, C. L., Munnich, A., Le Merrer, M. & Levy, N. (2003) Science 300, 2055.
Delbarre E, Tramier M, Coppey-Moisan M, Gaillard C, Courvalin J-C, et al. (2006) The truncated prelamin A in Hutchinson-Gilford progeria syndrome alters segregation of A-type and B-type lamin homopolymers. Human Molecular Genetic 15: 1113-1122.
Dimri, G. P., Lee, X., Basile, G., Acosta, M., Scott, G., Roskelley, C., Medrano, E. E., Linskens, M., Rubelj, I., Pereira-Smith, O. & al., e. (1995) Proceedings of the National Academy of Sciences 92, 9363-9367.
Djabali, K., Aita, V. M. and Christiano, A. M. (2001). Hairless is translocated to the nucleus via a novel bipartite nuclear localization signal and is associated with the nuclear matrix. Journal of Cell Science 114, 367-376.
Djabali, K., de Nechaud, B., Landon, F. and Portier, M.-M. (1997). aB-crystallin interacts with intermediate filaments in response to stress. Journal of Cell Science 110, 2759-2769.
Dreuillet, C., Tillit, J., Kress, M. and Ernoult-lange, M. (2002). In vitro and in vivo interactions between human transcription facotr Mok2 and nuclear lamin A/C. Nucleic Acids Research 30, 4634-4642.
Eriksson, M., Brown, W. T., Gordon, L. B., Glynn, M. W., Singer, J., Scott, L., Erdos, M. R., Robbins, C. M., Moses, T. Y., Berglund, P., Dutra, A., Pak, E., Durkin, S., Csoka, A. B., Boehnke, M., Glover, T. W. & Collins, F. S. (2003) Nature (London) 423, 293-298.
Fernandez, F. P., Mc Laren, A. T. and Slowie, D. F. (1992). Report on a case of Hutchinson-Gilford progeria, with special reference to orthopedic problems. European Journal of Pediatric Surgery 2, 378-382.
Fisher, D., Chaudhary, N. and Blobel, G. (1986). cDNA sequencing of nuclear lamins A and C reveals primary and secondary structure homology to intermediate filaments proteins. Proceedings of the National Academy of Sciences 83, 6450-6454.
Foisner, R. and Gerace, L. (1993). Integral membrane proteins of the nuclear envelope interact with lamins and chromosomes, and binding is modulated by mitotic phosphorylation. Cell 73, 1267-1279.
Francastel C, Schubeler D, Martin D I, Groudine M (2000) Nuclear compartmentalization and gene expression activity. Nature Reviews Molecular cell Biology 1: 137-143.
Frangioni, J. and Neel, B. (1993). Use of a general purpose mammalian expression vector for studying intracellular protein targeting: identification of critical residues in the nuclear lamin A/C nuclear localization sequence. Journal of Cell Science 105, 481-488.
Frolov, M. V. and Dyson, N.J. (2004). Molecular mechanisms of E2F-dependent activation and pRB-mediated repression. Journal of Cell Science 117, 2173-2181.
Fuchs E. (1990) Epidermal differentiation: The Bare essentials. Journal of Cell Biology 111: 2807-2814.
Fuchs E. (1995) Keratins and the skin. Annual review in cell developmental biology 11: 123-153.
Fuchs, E. & Weber, K. (1994) Annual Review of Biochemistry 63, 345-382.
Fukuchi, K., Katsuya, T., Sugimoto, K., Kuremura, M., Kim, H. D., Li, L. and Ogihara, T. (2004). LMNA mutation in a 45 year old Japanese subject with Hutchinson-Gilford progeria syndrome. Journal of Medical Genetic 41, e67.
Funayama R, Ishikawa F (2007) Cellular senescence and chromatin structure. Chromosoma.
Furukawa K, Hotta Y (1993) cDNA cloning of a germ cell specific lamin B3 from mouse spermatocytes and analysis of its function by ectopic expression in somatic cells. EMBO Journal 12: 97-106.
Gasparri, F., Mariani, M., Sola, F. and Galvani, A. (2004). Quantification of the proliferation index of human dermal fibroblast cultures with the arrayscan TM high-content screening reader. Journal of Biomolecular Screening, 232-243.
Georgatos, S. D. and Blobel, G. (1987). Lamin B constitutes an intermediate filament attachment site at the nuclear envelope. Journal of Cell Biology 105, 117-125.
Glynn, M. W. & Glover, T. W. (2005) Human Molecular Genetic 14, 2959-2969.
Goldman, R. D., Shumaker, D. K., Erdos, M. R., Eriksson, M., Goldman, A. E., Gordon, L. B., Gruenbaum, Y., Khuon, S., Mendez, M., Varga, R. & Collins, F. S. (2004) Proceedings of the National Academy of Sciences 101, 8963-8968.
Gruenbaum, Y., Goldman, R. D., Meyuhas, R., Mills, E., Margalit, A., Fridkin, A., Dayani, Y., Prokocimer, M. & Enosh, A. (2003) Internatianal Review cytology 226, 1-62.
Gutierrez, L., Magee, A. I., Marshall, C. J. and Hancock, J. F. (1989). Post-translational processing of p21Ras is a two-step and involves carboxyl-methylation and carboxy-terminal proteolysis. EMBO Journal 8, 1093-1098.
Holt, I., Clements, L., Manilal, S., Brown, S. C. & Morris, G. E. (2001) European Journal of Human Genetic 9, 204-208.
Horton, J. D. (2002). Sterol regulatory element-binding proteins transcriptional activation of lipid synthesis. Biochem. Soc. Trans. 30, 1091-1095.
Hozak P, Sasseville A M J, Raymond Y, Cook P R (1995) Lamin proteins form internal nucleoskeleton as well as a peripheral lamina in human cells. Journal of Cell Science 108: 635-644.
Hutchinson C J, Worman H J (2004) A-type lamins: guardians of the soma? Nature Cell Biology 6: 1062-1067.
Jagatheesan, G., Thanumalayan, S., Muralikrishna, B., Rangaraj, N., Karande, A. A. and Parnaik, V. K. (1999). Co-localization of intranuclear lamin foci with RNA splicing factors. Journal of Cell Science 112, 4651-4661.
Jolly, V. and Morimoto, R. I. (1999). Stress and the cell nucleus: dynamics of gene expression and structural reorganization. Gene Expression 7, 261-270.
Joyeux, M., Baxter, G. F., Thomas, D. L., Ribuot, C. and Yellon, D. M. (1997). Protein kinase C is involved in resistance to myocardial infarction induced by heat stress. Journal Molecular Cellular Cardiology 29, 3311-3319.
Krohne, G., Waisenergger, I. and Hoger, T. (1989). The conserved carboxy-terminal cysteine of nuclear lamins is essential for lamin association with the nuclear envelope. Journal of Cell Biology 109, 2003-2011.
Laemmli, U. K. (1970). Cleavage of structural proteins during the assembly of the head of bacteriophage T4. Nature (London) 227, 680-685.
Lassoued, K., Andre, C., Danon, F., Modogliani, R., Dhumeaux, D., Clauvel, J. P., Brouet, J. C. and Courvalin, J. C. (1992). Characterization of two human monoclonal IgM antibodies that recognize nuclear lamins. European Journal of Immunology 22, 1547-1551.
Lee, K. K., Haraguchi, T., Lee, R. S., Konjin, T., Hiraoka, Y. and Wilson, R. L. (2001). Distinct functional domains in emerin bind lamin A and DNA-bridging protein BAF. Journal of Cell Science 114, 4567-4573.
Lenher C F, Stick R, Eppenberger H M, Nigg E A (1987) Differential expression of nuclear lamin proteins during chicken development. Journal of Cell Biology 105.
Leung, G. K., Schmidt, W. K., Bergo, M. O., Gavino, B., Wong, D. H., Tam, A., Ashby, M. N., Michaelis, S, and Young, S. G. (2001). Biochemical Studies of Zmpste24-deficient Mice. Journal of Biological Chemistry 276, 29051-29058.
Li, Z., Cheng, H., Ledere, r. W. J., Froehlich, J. & Lakatta, E. G. (1997) Experimental and Molecular Pathology 64, 1-11.
Lin F, Worman H J (1995) Structural organization of the human gene (LMNB1) encoding nuclear lamin B1. Genomics 27: 230-236.
Lin, F. and Worman, H. J. (1993). Structural organization of the human gene encoding nuclear lamin A and nuclear lamin C. Journal of Biological Chemistry 268, 16321-16326.
Lin, F., Blake, D. L., Callebaut, I., Skerianc, I. S., Holmer, L., McBurnev, M. W., Paulin-Levasseur, M. and Worman, H. J. (2000). MANI, a inner nuclear membrane protein that shares the LEM domain with lamina-associated polypeptide 2 and emerin. Journal of Biological Chemistry 275, 4840-4847.
Liu B, Wang J, Chan K M, Tjia W M, Deng W, et al. (2005) Genomic instability in laminopathy-based premature aging. Nature Medicine 11: 780-785.
Lloyd D. J., Trembath, R. C. and Shackleton, S. (2002). A novel interaction between lamin A and SRBP 1: implication for partial lipodystrophy and other laminopathies. Human Molecular Genetic 11, 769-777.
Machiels B M, Zorenc A H G, Endert J M, Kuijpers H J H, Van Eys G J J M, et al. (1996) An alternative splicing product of the lamin A/C gene lacks exon 10. Journal of Biological Chemistry 271: 9249-9253.
Mallampalli, M. P., Huyer, G., Bendale, P., Gelb, M. H. & Michaelis, S. (2005) Proceedings of the National Academy of Sciences 102, 14416-14421.
Martelli, A. M., Bortul, R., Tabellini, G., Fantle, K., Cappellini, A., Bareggi, R., Monzolli, L. and Cocca, L. (2002). Molecular characterization of protein kinase C-alpha binding to lamin A. Journal of Biological Chemistry 86, 320-330.
Martelli, A. M., Tabellini, G., Bortul, R., Manzoli, L., Bareggi, R., Baldini, G., Grill, V., Zweyer, M., Narducci, P. and Cocca, L. (2000). Enhance nuclear diacylglycerol kinase activity in response to a mitogeniic stimulation of quescent Swiss with insulin-like growth factor 1. Cancer Research 60, 815-821.
Massague, J., Blain, S. W. and Lo. R. S. (2000). TGF-b signalling in growth control, cancer, and heritable disorders. Cell 103, 295-309.
McClintock D, Gordon L D, Djabali K (2006) Hutchinson-Gilford progeria mutant lamin A primarily targets human vascular cells as detected by an anti-Lamin A G608G antibody. Proceedings of the National Academy of Sciences 103: 2154-2159.
Mills, R. G. and Weiss, A. S. (1990). Does progeria provide the best model of accelerated ageing in humans? Gerontology 36, 84-98.
Mislow, J. M., Hoslaska, J. M., Kim, M. S., Lee, K. K., DSegura-Totten, M., Wilson, K. L. and McNally, E. M. (2002). Nesprin 1 a self-associates and binds directly to emerin and lamin A in vitro. FEBS Letter 525, 135-140.
Morimoto, R. I. (1998). Regulation of heat shock transcriptional response: cross talk between a family of heat shock factors, molecular chaperones, and negative regulators. Genes and Development 12, 3788-3796.
Mosachi, N., Nunez, S., Heard, E., Masako, N., Lin, A. W., Spector, D. L., Hannon, G. J. and Lowe, S. W. (2003). Rb-mediated heterochromatin formation and silencing of E2F target genes during cellular senescence. Cell 113, 703-716.
Narita M, Nunez, S, Heard E, Narita M, Lin A W, Hearn, S. A., et al. (2003) Rb-mediated heterochromatin formation and silencing of E2F target genes during cellular senescence. Cell 113: 703-716.
Navarro C L, Cadinanos J, De Sandre-Giovannoli A, Bernard R, Courrier S, et al. (2005) Loss of ZMPSTE24 (FACE-1) causes autosomal recessive restrictive dermopathy and accumulation of Lamin A precursors. Human Molecular Genetic 14: 1503-1513.
Navarro, C., De Sandre-Giovannoli, A., Bernard, R., Boccaccio, I., Boyer, A., Genevieve, D., Hadj-Rabia, S., Gaudy-MArqueste, C., Sillevis Smith, H., Vabres, P. et al. (2004). Lamin A and ZMPSTE24 (FACE-1) defects cause nuclear disorganization and identify Restrictive Dermopathy as a lethal neonatal laminopathy. Human Molecular Genetic.
Ostlund, C., Ellenberg, J., Hallberg, E., Lippincott-Schwartz, J. and Worman, H. J. (1999). Intracellular trafficking of emerin, the emery-Dreifuss muscular dystrophy protein. Journal of Cell Science 112, 1709-1719.
Ozaki, T., Saijo, M., Murakami, K., Enomoto, H., Taya, Y. and Sakiyama. (1994). Complex formation between lamin A and retinoblastoma gene product: identification of the domain on lamin A required for its interaction. Oncogene 9, 2649-2653.
Papamarcaki, T., Kouklis, P. D., Kreis, T. E. and Georgatos, S. D. (1991). Anti-idiotypic antibodies reveal a structural complementarity between nuclear lamin B and cytoplasmic intermediate filament epitopes. Journal of Biological Chemistry 266, 21247-21251.
Paradisi M, McClintock D, Boguslavsky R L, Pedicelli C, Worman H J, et al. (2005) Dermal fibroblasts in Hutchinson-Gilford progeria syndrome with the lamin A G608G mutation have dysmorphic nuclei and are hypersensitive to heat stress. BioMed Central Cell Biology 6: 27.
Pederson T. and Aebi, U. (2003). Actin in the nucleus: what form and what for? Journal of Structural Biology 140, 3-9.
Pendas, A. M., Zhou, Z., Cadinanos, J., Freije, J. M., Wang, J., Hultenby, K., Astudillo, A., Wernerson, A., Rodriguez, F., Tryggvason, K. & Lopez-Otin, C. (2002) Nature Genetics 31, 94-99.
Plasilova, M., Chattopadhyay, C., Pal, P., Schaub, N. A., Buechner, S. A., Mueller, H. J., Miny, P., Ghosh A. and Heinimann, K. (2004). Homozygous missense mutation in the lamin A/C gene causes autosomal recessive Hutchinson-Gilford progeria syndrome. Journal of Medical Genetic 41, 609-614.
Prufert, K., Vogel, A. & Krohne, G. (2004) Journal of Cell Science 117, 6105-6116.
Ralle, T., Grund, C., Franke, W. W. & Stick, R. (2004) Journal of Cell Science 117, 6095-6104. Rankin J, Ellard S (2006) The laminopathies: a clinical review. Clinical Genetics 70: 261-274.
Reddel C. J. and Weiss, A. S. (2004). Lamin A expression levels are unpertubated at the normal and mutant alleles but display partial splice step detection in Huchinson-Gilford progeria syndrome. Journal of Medical Genetic 41, 715-717.
Riemer, D., Karabinos, A. and Weber, K. (1998). Analysis of eight cDNAs and six genes for intermediate filament protein in the cephalochordate Brachiostoma reveals differences in the multigene families of lower chordates and vertebrates. Gene 211, 361-373.
Robert R A, Weber K, Osborn M (1989) Differential timing of nuclear lamin A/C expression in the various organs of the mouse embryo and the young animal: a developmental study. Development 105: 365-378.
Sasseville, A. M. & Raymond, Y. (1995) Journal of Cell Science 108, 273-285. Scaffidi P, Misteli T (2006) Lamin A-dependent nuclear defects in human aging. Science 312: 1059-1063.
Scaffidi, P. & Misteli, T. (2005) Nature Medicine 11, 440-445.
Schirmer, E. C., Florens, L., Guan, T., Yates, J. R. and Gerace, L. (2003). Nuclear membrane proteins with potential disease links found by subtractive proteomics. Science 301, 1380-1382.
Shumaker D K, Dechat T, Kohlmaier A, AAdam S A, Bozovsky M R, et al. (2006) Mutant lamin A leads to progressive alterations of epigenic control in premature aging. Proceedings of the National Academy of Sciences 103: 8703-8708.
Shumaker D K, Kuczmarski E R, Goldman R D (2003) The nucleoskeleton: lamins and actin are major players in essential nuclear functions. Current Opinion in Cell Biology 15: 358-366.
Sinensky M, Fantle K, Trujillo M, T. M, Kupfer A, et al. (1994) The processing pathway of prelamin A. Journal of Cell Science 107: 61-67.
Sinensky, M., Beck, L. A., Leonard, S. & Evans, R. (1990) Journal of Biological Chemistry 265, 19937-19941.
Sorrell J M, Caplan A I (2004) Fibroblast heterogeneity: more than skin deep. Journal of Cell Science 117: 667-675.
Stehbens, W. E., Delahunt, B., Shozawa, T. & Gilbert-Barness, E. (2001) Cardiovascular Pathology 10, 133-136.
Stehbens, W. E., Wakefield St. J., Gilbert-Barness, E., Oslon, R. E. & Ackerman, J. (1999) Cardiovascular Pathology 8, 29-39.
Steinert, P. M., Steven, A. C. & Roop, D. R. (1985) Cell 42, 411-420. Stick R, Hausen P (1985) Changes in the nuclear lamina composition during early development of Xenopus laevis. Cell 41: 191-200.
Stierle, V., Couprie, J., Ostlund, C., Krimm, I., Zinn-Justin, S., Hossenlopp, P., Worman, H. J., Courvalin, J. C. and Duband-Goulet, I. (2003). The carboxy-terminal region common to lamin A and C contains a DNA binding domain. Biochemistry 42, 4819-4828.
Sweeney, K. J. and Weiss, A. S. (1992). Hyaluronic acid in progeria and the aged phenotype? Gerontology 38, 139-152.
Takahashi, K., Sawasaki, Y., Hata, J. I., Mukai, K. and Goto, T. (1990). Spontaneous transformation and immortalization of human endothelial cells. In Vitro Cell Developemental Biology 25, 265.
Tang, K., Finley, R. L., Nie, D. and Honn, K. V. (2000). Identification of 12-lipoxygenase interaction with cellular protein by yeast two-hybrid screening. Biochemistry 39, 3185-3191.
Toth J I, Yang S H, Qiao X, Beigneux A P, Gelb M H, et al. (2005) Blocking protein farnesyltransferase improves nuclear shape in fibroblasts from humans with progeroid syndromes. Proceedings of the National Academy of Sciences 102: 12873-12878.
Van Gansen, P. & Van Lerberghe, N. (1988) Archives of Gerontology and Geriatics 7, 31-74.
Volz A, Korge B P, Compton J C, Ziegler A, teinert P M, et al. (2003) Physical mapping of a functional cluster of epidermal differentiation genes on chromosome 1 q21. Genomics 18: 92-93.
Vorburger, K., Kitten, G. T. and Nigg, E. A. (1989). Modification of nuclear lamin proteins by a mevalonic acid derivative occurs in reticulocyte lysates and requires the cysteine residue of the C-terminal CXXM motif. EMBO Journal 8, 4007-4013.
Wallis, C. V., Sheerin, A. N., Green, M. H. L., Jones, C. J., Kipling, D. and Faragher, R. G. A. (2004). Fibroblast clones from patients with Hutchinson-Gilford progeria can senesce despite the presence of telomerase. Experimental Gerontology 39, 461-467.
Wang H, Van Blitterswijk C A, Bertrand-de Hass M, Schuurman A H, Lamme E N (2004) Improved enzymatic isolation of fibroblasts for the creation of autologous skin substitutes. In Vitro Cell Developemental Biology 40: 268-277.
Wang, W. and Passaniti, A. (1999). Extracellular Matrix inhibits apoptosis and enhances endothelial cell differentitation by a NFkB-dependent Mechanism. Journal of Cell Biochemistry 73, 321-331.
Weber, K., Plessmann, U. & Traub, P. (1989) FEBS Letter 257, 411-414.
Weinberg, R. A. (1995). The retinoblastoma protein and cell cycle control. Cell 81, 323-330.
Worman H J, Bonne G (2007) “laminopathies”: a wide spectrum of human diseases. Experimental Cell Research 313: 2121-2133.
Wydner K L, McNeil J A, Lin F, Worman H J, Lawrence J B (1996) Chromosomal assignment of human nuclear envelope protein genes LMNA, LMNB1, and LBR by fluorescence in situ hybridization. Genomics 32: 474-478.
Yang, S. H., Bergo, M. O., Toth, J. I., Qiao, X., Hu, Y., Sandoval, S., Meta, M., Bendale, P., Gelb, M. H., Young, S. G. & Fong, L. G. (2005) Proceedings of the National Academy of Sciences 102, 10291-10296.
Young S G, Meta M, Yang A, Fong L G (2006) Prelamin A farnesylation and progeroid syndromes. Journal of Biological Chemistry 281: 39741-39745.
Yu C E, Oshima J, Fu Y H, Wijsman E M, Hisama F, et al. (1996) Positional cloning of the Werner's syndrome gene. Science 272: 258-262.
Zatrow M. S., Vlcek, S, and Wilson, K. L. (2004). Proteins that bind A-type lamins: integrating isolated clues. Journal of Cell Science 117, 979-987.

What is claimed is:

1. A peptide having amino acid sequence GAQSPQNC or having an amino sequence which is at least 75% identical to the amino acid sequence GAQSPQNC.

2. A mixture comprising at least two of the peptides of claim 1.

3. An antibody that specifically binds to the peptide of claim 1 or to a polypeptide comprising the peptide of claim 1.

4. The antibody of claim 2, wherein the antibody is a polyclonal antibody.

5. The antibody of claim 2, wherein the antibody is a monoclonal antibody.

6. A method for detecting a biological condition associated with a lamin A G608G mutation in a subject, the method comprising determining whether an antibody that specifically binds to lamin A G608G protein binds to a sample from the subject, wherein binding of the antibody indicates the presence of mutant lamin A G608G.

7. The method of claim 5, wherein the binding is detected by western blotting or immunohistochemistry.

8. A kit for detecting a biological condition associated with the presence of a lamin A G608G mutation in a subject, the kit comprising the antibody of claim 2.

9. A method for identifying an agent capable of inhibiting expression of mutant lamin A G608G protein in a cell, the method comprising:
a) contacting a cell expressing a mutant lamin A G608G protein with an agent,
b) determining whether the cell exhibits an increased or a decreased amount of the mutant lamin A G608G protein, wherein a decreased amount indicates that the agent inhibits expression of mutant lamin A protein.

10. The method of claim 9, wherein determining comprises immunohistochemistry using an antibody that specifically binds to the lamin A G608G protein.

11. The method of claim 9, wherein determining comprises Western blotting using an antibody that specifically binds to the lamin A G608G protein.

12. The method of claim 9, wherein determining comprises quantifying fluorescent signal.

13. The method of claim 9, wherein the cell is an endothelial cell, which expresses mutant lamin A G608G, or an HGPS fibroblast cell.

14. A method for identifying a protein that interacts with mutant lamin A G608G, the method comprising:
a) immunoprecipitating a protein interacting with mutant lamin A G608G, wherein immunoprecipitating is performed with the antibody of claim 2,
b) determining whether a protein immunoprecipitated with the antibody of claim 3 is identical or different compared to a protein immunoprecipitated with an antibody that immunoprecipitates lamin A, wherein the presence of a different protein in an immunoprecipitate with the antibody of claim 3 indicates interaction with mutant lamin 1 G608G or the absence of a protein in an immunoprecipitate with the antibody of claim 3 indicates loss of interaction with mutant lamin A G608G.

15. A nucleic acid encoding an inhibitory RNA molecule that inhibits the function of mutant lamin A G608G, wherein the nucleic acid consists essentially of a nucleic acid sequence as listed in any of SEQ ID NOS:6-48.

16. A composition comprising the nucleic acid of claim 15.

17. An expression vector that comprises a nucleic acid of claim 15.

18. A composition comprising inhibitory RNA of claim 15.

19. A method for inhibiting the function of mutant lamin A G608G, wherein the method comprises introducing the nucleic acid of claim 15 into a cell that expresses mutant lamin A G608G.

20. A method for treating a biological condition associated with a lamin A G608G mutation in a subject, the method comprising administering to a subject an effective amount of the nucleic acid of claim 15.

21. A method for treating a biological condition associated with a lamin A G608G mutation in a subject, the method comprising administering to a subject an effective amount of the composition of claim 16.

22. A method for treating a biological condition associated with a lamin A G608G mutation in a subject, the method comprising administering to a subject an effective amount of the expression vector of claim 17.

23. A method for treating a biological condition associated with a lamin A G608G mutation in a subject, the method comprising administering to a subject an effective amount of an inhibitor of mutant lamin A G608G.

24. The methods of claims 20-23, wherein the biological condition is a progeroid syndrome.

25. The methods of claim 23, wherein the inhibitor is a small molecule inhibitor, or an antibody.
